SEQUENCE LISTING (21 sequences)

SEQ ID NO: 1 - protein, 598 aa, Corallina officinalis
MGIPADNLQSRAKASFDTRVSAAELALARGVVPSLANGEELLYRNPDPENGDPSFIVSFTKGLPHDDNGAIIDPDDFLAFVRAINSGDEKEIADLTLGPARDPDTGLPIWRSDLANSLELEVRGWENSSAGLTFDLEGPDAQSIAMPPAPVLTSPELIAEIAELYLMALGREIEFSEFDSPKNAEYIQFAIDQLNGLEWFNTPAMLGDPPAEIRRRRGEVTVGNLFRGILPGSEVGPYLSQYIIVGSKQIGSATGGNKTLVSPNAADEFDGEIAYGSITISQRVRIATPGRDFMTDLKVFLDVQDAADFRGFESYEPGARLIRTIRDLATWVHFDALYEAYLNACLILLANRVPFDPNIPFQQEDKLDNQDVFVNFGDAHVLSLVTEVATRALKAVRYQKFNIHRRLRPEATGGLISVNKIAAEKGESVFPEVDLAVEELEDILEKAEISNRKQNIADGDPDPDPSFLLPQAFAEGSPFHPSYGSGHAVVAGACVTILKAFFDSNFQIDQVFEVDKDEDKLVKSSFKGTLTVAGELNKLADNIAIGRNMAGVHYFSDQFESILLGEQVAIGILEEQSLTYGENFFFNLPKFDGTTIQI

SEQ ID NO: 2 - protein, 598 aa, Corallina pilulifera
MGIPADNLQSRAKASFDTRVAAAELALNRGVVPSFANGEELLYRNPDPDNTDPSFIASFTKGLPHDDNGAIIDPDDFLAFVRAINSGDEKEIADLTLGPARDPETGLPIWRSDLANSLELEVRGWENSSAGLTFDLEGPDAQSIAMPPAPVLTSPELVAEIAELYLMALGREIEFSEFDSPKNAEYIQFAIDQLNGLEWFNTPAKLGDPPAEIRRRRGEVTVGNLFRGILPGSEVGPYLSQYIIVGSKQIGSATVGNKTLVSPNAADEFDGEIAYGSITISQRVRIATPGRDFMTDLKVFLDVQDAADFRGFESYEPGARLIRTIRDLATWVHFDALYEAYLNACLILLANGVPFDPNLPFQQEDKLDNQDVFVNFGSAHVLSLVTEVATRALKAVRYQKFNIHRRLRPEATGGLISVNKIAPQKGESIFPEVDLAVEELGDILEKAEISNRKQNIADGDPDPDPSFLLPMAFAEGSPFHPSYGSGHAVVAGACVTILKAFFDSGIEIDQVFEVDKDEDKLVKSSFKGTLTVAGELNKLADNIAIGRNMAGVHYFSDQFESLLLGEQVAIGILEEQSLTYGENFFFNLPKFDGTTIQI

SEQ ID NO: 3 - protein, 598 aa, Corallina pilulifera
MGIPADNLQSRAKASFDTRVAAAELALNRGVVPSFANGEELLYRNPDPDNTDPSFIASFTKGLPHDDNGAIIDPDDFLAFVRAINSGDEKEIADLTLGPARDPETGLPIWRSDLANSLELEVRGWENSSAGLTFDLEGPDAQSIAMPPAPVLTSPELVAEIAELYLMALGREIEFSEFDSPKNAEYIQFAIDQLNGLEWFNTPAKLGDPPAEIRRRRGEVTVGNLFRGILPGSEVGPYLSQYIIVGSKQIGSATVGNKTLVSPNAADEFDGEIAYGSITISQRVRIATPGRDFMTDLKVFLDVQDAADFRGFESYEPGARLIRTIRDLATWVHFDALYEAYLNACLILLANGVPFDPNLPFQQEDKLDNQDVFVNFGSAHVLSLVTEVATRALKAVRYQKFNIHRRLRPEATGGLISVNKIAAQKGESIFPEVDLAVEELGDILEKAEISNRKQNIADGDPDPDPSFLLPMAFAEGSPFHPSYGSGHAVVAGACVTILKAFFDSGIEIDQVFEVDKDEDKLVKSSFKGTLTVAGELNKLADNIAIGRNMAGVHYFSDQFESLLLGEQVAIGILEEQSLTYGENFFFNLPKFDGTTIQI

SEQ ID NO: 4 - protein, 568 aa, Corallina pilulifera
MGIPADNLQSRAKASFDTRVAAAELALARGVVPSFANGEELLYRDFLAFVRAINSGDEKEIADLTLGPARDPETGLPIWRSDLANSLELEVRGWENSSAGLTFDLEGPDAQSVAMPPAPVLMSPELIAEMAELYLMALGRDIEFSEFESPKNAAFIRSAIERLNGLEWFNTPAKLGDPPAEIRRRRGEVTVGNLFRGILPGSEVGPYLSQYIIVGSKQIGSATVGNKTFVSPNAADEFDGEIAYGSITISQRVRIATPGRDFMTDLKVFLDVQDGADFRGFESYEPGARLIRTIRDLATWVHFDALYEAYLNACLILLANGVPFDPNLPFQQEDKLDNHDVFVNFGSAHVLSLVTEVATRALKAVRYQKFNIHRRLRPEATGGLISVNKKSFLAGSDIIFPEVSELVEELSSILDDVAESNEKQNRADGIVSPDKSFLLPMAFAEGSPFHPSYGSGHAVVAGACVTILKAFFDANFQIDKVFEVDTDEDKLVKSSFKGTLTVAGELNKLADNVAIGRNMAGVHYFSDQFESLLLGEQIAIGILEEQSLTYGENFFFNLPKFDGTTIQI

SEQ ID NO: 5 - protein, 596 aa, Corallina officinalis
MGIPADNLQSRAKASFDTRVAAAELALARGAVPSFANGEELLYRNSETGDPSFIGSFTKGLPHDDNGAIIDPDDFLAFVRAINSGDEKEIAALTLGPARDPETGLPIWRSDLANSLDLEVRGWENSSAGLTFDLEGPDAQSVAMPPAPVLTSPELIAEMAELYLMALGRDIEFSEFDSPKNAAFIRSAIERLNGLEWFNTPAKLGDPPAEIRRRRGEVTVGNLFRGILPGSEVGPYLSQFIIVGSKQIGSATVGNKTLVSPNAADEFDGEIAYGSITISQRVRIATPGRDFMTDLKVFLDVQDAADFRGFESYEPGARLIRTIRDLATWVHFDSLYEAYLNACLILLANGVPFDPNLPFQQEDKLDNQDVFVNFGSAHVLSLVTEVATRALKAVRYQKFNIHRRLRPEATGGLISVNKNAFLKSESVFPEVDVLVEELSSILDDSASSNEKQNIADGDVSPGKSFLLPMAFAEGSPFHPSYGSGHAVVAGACVTILKAFFDANFQIDQVFEVDTDEDKLVKSSFPGPLTVAGELNKLADNVAIGRNMAGVHYFSDQFESLLLGEQIAIGILEEQSLTYGENFFFNLPKFDGTTIQI

SEQ ID NO: 6 - protein, 625 aa, Neopyropia yezoensis
MAGPTASGTARRNEAFDKRVAAATVARERVHPPHAANGEEYEYRATFDRHPAVAAAKAAAAGASREASNGDPGATDGGATAAGAPPSSQSQVQGPSHIGSFTKGLQHNLSTGLLEDPAAFRAFVRAIDSGDPRDFVDVPIADRFTRPPTRGVRSWESASAGLAFDLQGPDAQALTMPPAPRLGSAELVAEMIEVYAMALARDVPFATAEWAAAKPIAAAVRELNATRWHGAAATDRELGLSPAAAARRRVTHFTPATVFRGVGRGVADGPYLSQFLLLGNAGVGGSSGAPTDGFITYGANRVDLRVPVATPGVDYLTDWESFVDVQNGADTRGTETYVPAADGTKTRFIATPRDLATYVHYDALYQAYLNAALWLLAEGVPFDAGLPWTAPDEVDHQQGFATFGGPHLLTLVTEVATRALKAVRFQKFNVHRRLRPEAVGGLLDRLPVAPGPLAPVAPLAEEAAETLRRVADANAAAAAGGSGAGAPVTHSVLLPMAFPEGSPMHPSYGAGHATVAGACVTVLKGWFDAGHALGTTAYEATPDGRCLREVSTGGVALTVEGELNKLAANISIGRNWAGVHYYSDYIESVQLGEAVAIGILEEQKLTYGENWSMTVPLTDGTTVRI

SEQ ID NO: 7 - protein, 577 aa, Asparagopsis taxiformis
TRRQTALVKRIAAADLAANRHHPHHVRNADETTYKLPNHVCSYISAFTKGLPHHLDGRLVSPHHFQLFIHAVRTGHVKDIAAIPLGPPHSSSFRSGIAKDAAVRGWESMAAGNTFDLQGPDAQAVTMPPCPRLNSHELVTEMTELYYMALLRDVPFTHFNKHHLVQSAVYDMNHTHWTKSNRCCLPRPERRRHRSKFTTDNIFRGVTPGDITGPYISQFLLVGTKGLADVHEETEGYVQYGATRIDQRVRTVQPKIDYMTTWAAYLDVQNGADLRRHSAYAEPPNYRFICTPRDLATYVHYDALYQAYLNACIILLDINAPYDSGLPFTADDAVDKQQGFATFGAPHVLTLVTEVATRALKAVRFQKYAVHRRLRPEAVGGLLEQYRRYGGGHSDLAYIIRPIRHLADSLSSDLMSMVAKENGRQNSLIRDNGHPRSHDEGSDDTHLLSMAYAEGSPMHPSYGAGHATVAGACVTILKAFFDHTFKLPFAYVSSSDGRKLKTVKLSKKLTVEDELNKLAANISIGRSWAGVHYYSDYVESIRLGEEVAIGMLKEQKLTYSEKFTMTIPKFDGSVIEI

SEQ ID NO: 8 - protein, 584 aa, Asparagopsis taxiformis
TRRQTALVKRIAAADLAANRHHPLHVRNADETTYKLPNHVCSYISAFTKGLPHHLDGRLVSPHDFLAFVRAINSGDEKEIAALTLGPARDPETGLPIWRSDLANSLDLEVRAWESMAAGNTFDLQGPDAQAVTMPPCPRLNSHELVTEMTELYYMALLRDVPFTHFNKHHLVQSAVHDMNHTHWTRSNRCCLPRPERRRHRSKFTTDNIFRGVTPGDITGPYISQFLLVGTKGLGDAHEETEGYVQYGATRIDQRVRTVEPKIDYMTTWAAYLDVQNGADLRRHSTYAKPPSYRFISTPRDLATYVHYDALYQAYLNACIILLDINAPYDGGLPFTADDAVDKQQGFATFGAPHVLTLVTEVATRALKAVRFQKYAVHRRLRPEAVGGLLEQYRRYGGGHSDLAYIIRPIRHLADSLSSDLMFMVAKENGRQNSSITDNGHPRSHDEGSDDTHLLSMAYAEGSPMHPSYGAGHATVAGACVTILKAFFDHTFKLPFAYVSSSDGRKLKTVKLSRKLTVEDELNKLAANISIGRSWAGVHYYSDYVESIRLGEEVAIGMLKEQKLTYSEKFTMTIPKFDGSVIEI

SEQ ID NO: 9 - protein, 613 aa, Chondrus crispus
ISPRQQEAYDIRVAAATLALARDRKRKVFEPNGEEDRYFRPNSQILSYLGSYTKGLPHNRSTALVTSPNHFVRFVQAIDSGEPADFIKVPLGPRPALEFTTEEECIAANLFKSGIATRNDSFDPACLRAWESMGAGLVFDLEGPDAQAVGMPPAPTLDSDELVAEISELYWMALLRDVRFTDFTNPGPIRRAINTLNRTTWIRAARDPPDSLTPQERARLRGPFNPSSIFRGTLPGDDVGPYLSQFLLVGTEGIGGAQDRADGFISYGAHRIDQRVRHATKELDYMTSWEAFLDVQNGADVAGRDTFESGSDAFRFITTPRDLATYVHFDALYQAYLNACLILLDNKVKFDQGIPMGEPDFNGNQGIPFQDPDFKDHQRGFAHFGGPHILSLVTEVATRALKAVRFQKFNTHRRLRPEAVGGLIERFNSNPDDPQFQDVKPLFEALDEDMLRRVASHNREQNERSDFGMPRADDFNPAGDTLETMLLPMAFPEGSPMHPSYGAGHATVAGACVTVLKAFFQHDATLDFCYVPSDDGSRLDDASHTLNKKLTVEGELNKVCSNISIGRNWAGVHYFTDYIESILLGEQIALGILEEQMLTFPETFTMTVPLFSG

SEQ ID NO: 10 - protein, 596 aa, Chondrus crispus
ARRQEAFQTRVRAARLARDRDHQVFEPNGEEDRYFRPNSQIFSYLGSYTKGLPHNRRTALITSPNHFVRFVRAIDSGEPADFIKVPLGPRPALQFESEEECRAANLFKSGIAGRNNTFPSSCLRAWESMGAGLVFDLEGPDAQAVGMPPAPTLDSDELVAEISEVYWMALLRDVRFTDFTNPGPIGRAINTLNKTAWIRAARDPPDSLTPQERARLRGPFNPSNIFRGTLPGDDVGPYLSQFLLVGTKGIGGAQDMADGFISYGAHRIDQRVRHARKELDYMTSWEAFLDVQNGADVTGRDTFESGSDAFRFITTPRDLATYVHFDALYQAYLNACLIMLDSGVRFDKGIPFGEPDFKDHQRGFAHFGGPHILSLVTEVATRALKAVRFQKFNTHRRLRPEAVGGLIERFNSNPDDPQFQDVKPLFEALDEDMMRRVARHNREQNQGSDFGMPRADDFNPAGDTLETMLLPMAFPEGSPMHPSYGAGHATVAGACVTVLKAFFQHDTELDFCFVPSDDGSRLVDASHNMNKKLTVEGELNKVCSNISIGRNWAGVHYFTDYIESILLGEQIALGILEEQMLTFPETFTMTVPLFSG

SEQ ID NO: 11 - protein, 599 aa, Chondrus crispus
QARKQEAFQTRVAAARLARDRDHQVFEPNGEEDRYFRPNSQILSYLGSYTKGLPHNRRTALVTSPNHFVRFVRAIDSGEPADFIKVPLGPRPALEFESEEECRAANLFKSGIAGRNNTFPSSCLRAWESMGAGLVFDLEGPDAQAVGMPPAPTLDSDELVAEISEVYWMALLRDVRFTDFTNPGPIGRAVNTLNRTTWIRAARDPPDSLTPQERARLRGPFNPSNIFRGTLPGDDVGPYLSQFLLVGTEGIGGAQDRADGFISYGAHRIDQRVRHATKELDYMTSAAWEAFLDVQNGADVAGRDTFESGSDAFRFITTPRDLATYVHFDALYQAYLNACLIMLDSGVKFDKGIPFGEPDFKDHQQGFAHFGGPHILSLVTEVATRALKAVRFQKFNTHRRLRPEAVGGLIERFNSNPDDPQFQDVKPLFEALDEDMMRRVARHNREQNQGSDFGMPRADDFNPAGDTLETMLLPMAFPEGSPMHPSYGAGHATVAGACVTVLKAFFQHDTELDFCFVPSNDGSRLVDASHNMNKKLTVEGELNKVCSNISIGRNWAGVHYFTDYIESILLGEQIALGILEEQMLTFPETFTMTVPLFSG

SEQ ID NO: 12 - protein, 604 aa, Gracilariopsis chorda
NRFTEAYNVRLEAATLAKNRPHPIHLANTEELRYRRPFDCEFPGQPSHIASYTKGLPHDPKTGLLIKPNDFQTFIRALDAGDEFSIREVPLGPQIPEPRFISGIASTPKADGSPTDVRAWESMAAGNTYGLEGPDAQAVTMPPAPALDSKELVYEISESYWMALVRDVPFTRFDDDVVRDAVRSLNQTEWIRFLAKGGPLPPSLSDAQRKRLRKPFTIDTVFRGVTKGDLEGPYLSQFMLIGNSGIANGNKIEDGFVQYGAHRVDQKVRAVEDGRDYMTTFKSFVDVQNGADVRGREVYSDGFRFITTPRDLTTYVHFDALYQAYLNACLILLGMKVPFDKGLPFQLDDDVDKQQTFATFGGPHILSLVTEVATRALKAVRFQKYNVHRRLRPEAVGGLIEYYHNNPDSEIVAGVKPLYEALDKDMLNRVSMHNESQNKLCDGDVPRMSDFEDGESSATRLLPMAFPEGSPMHPSYGAGHAAVAGACVTVLKAFFDHGYELRMLDENGNMVPFAFVPTRDGQSLKNVISEYGNKPLTVEGELNKVCSNISIGRNWAGVHYFTDYRESITLGEKIAIGILEEQKLTYGENFSMTVPLFDGTVYRI

SEQ ID NO: 13 - protein, 604 aa, Gracilariopsis chorda
NRFTEAYNVRLEAATLAKNRPHPIHLANTEELRYRRPFDCEFPGQPSHIASYTKGLPHDPKTGLLIKPNDFQTFIRALDAGDEFSIREVPLGPQIPEPRFISGIASTPKADGSPTDVRAWESMAAGNTYDLEGPDAHAVTMPPAPALDSKELVYEISESYWMALVRDVPFTRFDDDVVRDAVRSLNQTEWIRFLAKGGPLPPSLSDAQRKRLRKPFTIDTVFRGVTKGDLEGPYLSQFMLIGNSGIANGNKIEDGFVQYGAHRVDQKVRAVEDGRDYMTTFKSFVDVQNGADVRGREVYSDGFRFITTPRDLATYVHFDALYQAYLNACLILLGMKVPFDKGLPFQLDDDVDKQQTFATFGGPHILSLVTEVATRALKAVRFQKYNVHRRLRPEAVGGLIEYYHNNPDSEIVAGVKPLYEALDKDMLNRVSMHNESQNKLCDGDVPRMSDFEDGESSATRLLPMAFPEGSPMHPSYGAGHAAVAGACVTVLKAFFDHGHELRMPDENGNMVPFAFVPTRDGQSLKNVISEYGNKPLTVEGELNKVCSNISIGRNWAGVHYFTDYRESITLGEKIAIGILEEQKLTYGENFSMTVPLFDGTVYRI

SEQ ID NO: 14 - protein, 604 aa, Gracilariopsis chorda
NRFTEAYNVRLEAATLAKNRPHPIHLANTEELRYRRPFDCEFPGQPSHIASYTKGLPHDPKTGLLIKPNDFQTFIRALDAGDEFSIREVPLGPQIPEPRFISGIASTPKADGSPTDVRAWESMAAGNTYDLEGPDAHAVTMPPAPALDSKELVYEISESYWMALVRDVPFTRFDDDVVRDAVRSLNQTEWIRFLAKGGPLPPSLSDAQRKRLRKPFTIDTVFRGVTKGDLEGPYLSQFMLIGNSGIANGNKIEDGFVQYGAHRVDQKVRAVEDGRDYMTTFKSFVDVQNGADVRGREVYSDGFRFITTPRDLTTYVHFDALYQAYLNACLILLGMKVPFDKGLPFQLDDDVDKQQTFATFGGPHILSLVTEVATRALKAVRFQKYNVHRRLRPEAVGGLIEYYHNNPDSEIVAGVKPLYEALDKDMLNRVSMHNESQNKLCDGDVPRMSDFEDGESSATRLLPMAFPEGSPMHPSYGAGHAAVAGACVTVLKAFFDHGYELRMPDENGNMVPFAFVPTRDGQSLKNVISEYGNKPLTVEGELNKVCSNISIGRNWAGVHYFTDYRESITLGEKIAIGILEEQKLTYGENFSMTVPLFDGTVYRI

SEQ ID NO: 15 - protein, 599 aa, Gracilaria domingensis
SRDNKSYGVREQAALLAKSRPHPIHVANAEELRYRIPFDGSAIQGMPSHIASYTKGLPHDLETGVLVNPNDFQTFIRAMDSGDKFSIKEIPLGPQIPEPRFTSGIAGPGTDVRAWESMGAGNTFDLEGPDAQSVTMPPAPTLDSDELVTEITECYWMALLRDISFSEFSTNNLVCEAVESLNKTRWVELSKHGATVPASLTVPERRRLRGPFTSETVFRGTTRGDDVGPYLSQFLLVGNSGIAGGNKISDGQIQYGAHRVDQRVRVAEKGRDFMTTWQSFMDVQNGADVRGREVYDNDVPFRFIATPRDLATYVHFDALYQAYLNACLILLGMKVPFDKGLPFQLDDDVDRQQTFATFGAPHILSLVTEVATRALKAVRFQKFNVHRRLRPEAVGGLIERFLEDPASDLFSGVQNLCEQLDESLVKTVRKHNKKQNEKSDNGVPRNDDKGAGRTMLMPMAFPEGSPMHPSYGAGHATVAGACVTVLKAFFDHGYELRMKDDEGKMVPFAFVANEDGSELVNEIEKYGNKALTVEGELNKVCSNICIGRNWAGVHYFSDYRESIEIGEKIAIGVLEEQKLTYGENFSMSVPLFDGSVYRI

SEQ ID NO: 16 - protein, 599 aa, Gracilaria changii
RFTKAYAVREQAALMAKSRPHPLHVSNNEELRYRIPTSGEPSHIGSYTKGLPHDRNTGVLSNPDDFQSFIRAMDAGDQFSIRDVPLGPQNEQQAFQSGIAKGVSARAWESMAAGLTYDLEGPDAQSVTMPPAPALDSDELLTEVTELYWMALLRDVPFSEFNGDKNNDIQEAVDSLNSTPWVQIAKNGVVPTTLTDAERVRLRGPFTVDNVFRGTTRGDRDGPYLSQFMLIGNTGIGNGNRIEDGFIRYGAHRVDQRVRVAEMGRDFMTTWSAFVDVQNGADVRGREVYNNDVRYRFISTPRDLATYVHFDALYQAYLNACLILLGMRVPFDKGLPFQREDDIDHQQTFATFGAPHILSLVTEVATRALKAVRFQKFNVHRRLRPEAVGGLLHRYMQETTNDLYRGAKQLAENLDANLLDKVKRHNAKQNMKSDGDAPRANDLGECEDADEITLLPMAFPEGSPMHPSYGAGHATVAGACVTILKAFFDHGYELRMLDEDGETMIPFAFVPTADGSGLENRIGEYRNLTALTVEGELNKVCSNICIGRNWGGVHYFSDYRESIRVGEQVAIGLLEEQKLTYGENFSMTVPLFDGSVHRI

SEQ ID NO: 17 - protein, 601 aa, Chondrus crispus
ATPCATRQQQALELRRAAAQLAHDRAHPAHVSNGSEELHLHPKTGAPLLLANFTKGLPHERDTGLAADAAHYDLFVAAVHSGLPDDIRAIPLGPQAAPGVRLPRSLRFRSGIARSSHLFPHTDVRAWESMAAGNAFDLQGPDAQAVTMPPAPALASPELALEMTENYWMALLRDVRFSQFAADPTVAAAMESMNATEWVALACGDASAVAEEERKRLRGPFTAQNVFRGNTQGDCKGPYISQFLLSGSAGLGQVNSGADGYIQYGAMRMDQRVRVALEGLDYMTTWESWLDVQNGADLRGRELYEDGERFRFICTPRDLATYVHYDALYQAYLNACIIMLNKKIPFDKGLPFQKDDDIDKQQGFALFGPPHILTLCTEVATRALKGIRFQKYNVHRRLRPEAVGGRVERYHHNCEDPLFADVKPLYDALDKDMLERVAAHNAEQNAKDDFGNSRFEDYSPTGSSGRTYLLPMAFPEGSPMHPAYGAGHAAVAGACTTILKAFFDHEHELDFAYVPTADGSKLEDVVDSLGEKLTVEGELNKVCSNISVGRNWAGVHYFTDYRESIILGEKIALGLLEEQKLTYAEEFSMTIPLFDGSTVTI

SEQ ID NO: 18 - protein, 583 aa, Asparagopsis taxiformis
NRAEVAFKVRVSAAELARARGSPAHLSNNSESRFRNPDGTRSLLANFTKGLPHIKETALVESAIDYDRFVRAIDSGDPRDFADLPLGPQGVEPRFTSGIASDPEVGTRAWESGGAGLVFDLEGPDAQAVTMPPAPELDSDELVAEVTECYWMSLLRDVPFPTFESNSHIQAAAESINNTQWIKFKNNPPAHLTAAERSRLRGPVTTANVFRGITPGDEVGPYLSQFLLVGTTGIANGNEVGDGFIQYGGMRMDQRVRVAKPHIDYMTTFGAYLDVQNAANVSGRELYKEEEPRFRFIHTPRDLATYVHFDALYQAYLNACIILLDIGAPFDSGIPFQLDNDIDKQQGFATFGGPHILSLVTEVATRALKAVRFQKFNVHRRLRPEAIGARVDRYCATKAPEFAGAAKLSEALDKELLQKVHDHNKKQNLLSDRGNPRANDFNPDGEVSEGNLLMPMAFPEGSPMHPAYGAGHATVAGACVTVLKAFFDGGYRLPFCYITDEDGTGLQAVEIDEPLTVDGELNKICSNISIGRNWAGVHYFTDYIESIRIGEEIAIGILQEQKLTFSENFSMTLNKFDGSTIRI

SEQ ID NO: 19 - protein, 583 aa, Asparagopsis taxiformis
NRAEVAFKVRVSAAELARARGSPTHLSNNSESRFRNPDGTRSLLANFTKGLPHTKETALVVSAIDYDSFVRAIDSGDPRDFADLPLGPQGVEPRFTSGIASDPEVGTRAWESGGAGLVFDLEGPDAQAVTMPPAPELDSDELVTEVTECYWMSLLRDVPFPTFDSDPHIQSAAESINNTQWIQFKNNPPAHLTAAERSRLRGPITTANVFRGITPGDEVGPYLSQFLLVGTTGIANGNEVGDGFIQYGGMRMDQRVRVAKSHIDYMTTFGAYLDVQNAANVSGRELYNEEEPRFRFIHTPRDLATYVHFDALYQAYLNACIILLDIGAPFDSGIPFQLDNDIDKQQGFATFGGPHILSLVTEVATRALKAVRFQKFNVHRRLRPEAIGARVDRYCATKAPEFAGAAKLSEALDKKLLQKVHDHNKKQNLLSDRGNPRANDFNPDGDVSKGNLLMPMAFPEGSPMHPAYGAGHATVAGACVTVLKAFFDGGYRLPFCYITDENGTGLKAVEIDEPLTVDGELNKICSNISIGRNWAGVHYFTDYIESIRIGEEIAIGILQEQKLTFSENFSMTLNKFDGSTIRI

SEQ ID NO: 20 - protein, 584 aa, Asparagopsis taxiformis
EERRQNALEIRIQAAKLAKKRDHPTHKANGDEDRYPETLIGSFTKGLPHEKETGLLSNPADFADFVRAINTGAIKDMRRLKLGIDEDPRFISGIASKGDKHPFADTRAWESMAAGLTYDLEGPDAQAVTMPPAPKLDSDELVTEITESYWMALLRDVPFTEFERDGNTAAAAASISRTRWVQYNEQPSQRPSTLTDEEIARLRGPYTKKNVFRGVTNGENVGPYLSQFLLVGTKGIGDAQQVSDGYVQYGGMRMDQRVRVAVPKRDYMTTWASWLDVQNAGDLRGREIYDDDTPFRFITTPRDLATWVHFDALYQAYLNACIILLDIKAPFDPHIPFQADDDVDKQQGFATFGGPHILSLCTEVATRALKAVRFQKYNLHRRLRPEAIGGLVERFKKTNGDPKFAPVKKLVNDLDGDMLRRVEQHNCEQNKLSDDGHARREDYSPEGESSQSYLLPMAFPEGSPMHPSYGAGHATVAGACVTVLKAFFDHEYELDFCYVPTTDGKRLEKVNINEKLTVEGELNKLCANISIGRNWAGVHYYSDYFESIKVGEEIAIGILQEQKLTYGEDFFMTLPKFDGEKIRI

SEQ ID NO: 21 - protein, 537 aa, Asparagopsis taxiformis
EERRQNALDIRIQAARLAKKRDHPTHEANGDEDRYPETLIGSFTKGLPHEKETGLLSNPAAWESMAAGLTYDLEGPDAQAVTMPPAPKLDSDELVTEITESYWMALIRDVPFTEFERDGNTAAAAASISRTRWVQFNEQPSQRPSTLTDEEIARLRGPYTKKNVFRGVTIGENVGPYLSQFLLVGTKGIGDVQQISDGYIQYGSMRMDQRVRVAAPKRDYMTTWASWLDVQNAGDLRGREIYDDDTPFRFITTPRDLATWVHFDALYQAYLNACIILLDIKAPFDPHIPFQADDDVDKQQGFATFGGPHILSLCTEVATRALKAVRFQKYNVHRRLRPEAIGGLVERFKKTNGDAKFAPVKKLVNDLDGDMLRRIEQHNCEQNKLSDDGHARRDDYSPEGESNQTYLLPMAFPEGSPMHPSYGAGHATVAGACVTVLKAFFDHEYELDFCYVPTTDGKRLEKVNINEKLTVEGELNKLCANICIGRNWAGVHYYSDYFESIKVGEEIAIGILQEQKLTYGEDFFMTLSKFDGEKIRI

(19) United States
(12) Patent Application Publication
(10) Pub. No.: US 2025/0346931 A1
(43) Pub. Date: Nov. 13, 2025

(54) METHODS FOR PRODUCING HALOGENATED COMPOUNDS

(71) Applicant: BIGELOW LABORATORY FOR OCEAN SCIENCES, East Boothbay, ME (US)

(72) Inventors: Stephen D. Archer, Nobleboro, ME (US); David Emerson, Alna, ME (US); José A. Fernández-Robledo, Damariscotta, ME (US); Athanasios Foukis, Damariscotta, ME (US); Nichole N. Price, Edgecomb, ME (US)

(21) Appl. No.: 18/860,364

(22) PCT Filed: Apr. 26, 2023
(86) PCT No.: PCT/US2023/020075

§ 371 Date: Oct. 25, 2024

Related U.S. Application Data
(60) Provisional application No. 63/335,010, filed on Apr. 26, 2022.
(60) Provisional application No. 63/451,399, filed on Mar. 10, 2023.

(51) Int. Cl. C12P 7/28; C12N 1/18; C12N 1/20; C12N 9/08; C12R 1/19; C12R 1/685; C12R 1/84; C12R 1/865; G01N 33/00

(52) U.S. Cl. CPC C12P 7/28; C12N 1/18; C12N 1/20; C12N 9/0065; C12Y 111/01802; C12Y 111/01806; G01N 33/0004; C12R 2001/19; C12R 2001/685; C12R 2001/84; C12R 2001/865

(57) ABSTRACT

The present disclosure features a method of modulating production of a small halogenated organic compound with a peroxidase enzyme, and related compositions and methods of use thereof.

### CLAIM OF PRIORITY

This application claims priority to U.S. Application No. 63/335,010, filed Apr. 26, 2022; and U.S. Application No. 63/451,399, filed Mar. 10, 2023; the entire contents of each of the foregoing applications are incorporated herein by reference.

### BACKGROUND

There are growing concerns about the greenhouse gas emissions from animal agriculture. A large portion of these emissions can be attributed to biogenic enteric methane (CH₄) emissions from all domesticated ruminants (3.2% of total U.S. emissions; EPA, 2019). Small halogenated organic compounds, such as chloroform, bromochloromethane, and 2-bromoethane sulfonate, have long been known to act as inhibitors of enteric methane production (Hristov, A. N. et al. J Anim Sci (2013) 91(11):5045-69). These small organic halogenated compounds can competitively inhibit the activity of methyl-coenzyme M reductase (mMCR), the enzyme that catalyzes the final step of CH₄ synthesis by methanogens found in the rumen (Wood, J. M. et al. Biochemistry (1968) 7(5):1707-1713; Ferry, J. G. Annu Rev Microbiol (2010) 64:3117-3126). However, animal and human safety and environmental concerns prohibit using these small halogenated organic compounds from being applied directly as livestock feed additives. To circumvent this issue, researchers have discovered that halogen-rich red seaweeds of the Asparagopsis genus are a natural source of small halogenated organic compounds that are CH₄ inhibitors (Machado L. et al, J Appl Phycol (2016) 28(5):3117-3126). Although many seaweeds generate halogenated compounds, Asparagopsis is unusual in possessing gland cells that accumulate very high concentrations of primarily bromoform (CHBr₃). A series of independent in vivo trials demonstrated substantial reductions of between 50-80% in CH₄ production when Asparagopsis was included in the diets of sheep, dairy, and beef cattle (Li X., et al. Anim Prod Sci (2018) 58(4):681-688). Despite research efforts to mass-produce Asparagopsis, growth of this seaweed is unlikely to match the scale of future demands for feed additives in the dairy and beef industries. As such, there is a need for developing scalable alternatives for reducing methane production.

### SUMMARY

The present disclosure features peroxidase enzymes and related compositions, as well as methods of modulating production of a small halogenated organic compound with said peroxidase enzymes. In an embodiment, the methods described herein comprise (i) providing a small organic compound (e.g., acetone or acetyl acetone); (ii) contacting the small organic compound with a peroxidase (e.g., a VHPO) to form a reaction mixture under conditions sufficient to produce a small halogenated organic compound; and/or (iii) evaluating the small halogenated organic compound produced. In an embodiment, the method features (i). In an embodiment, the method features (ii). In an embodiment, the method features (iii). In an embodiment, the method features each of (i) and (ii). In an embodiment, the method features each of (i) and (iii). In an embodiment, the method features each of (ii) and (iii). In an embodiment, the method features each of (i)-(iii). In an embodiment, the modulating comprises increasing the production of the small halogenated organic compound. In an embodiment, the method comprises modulating production of a plurality of small halogenated organic compounds. In an embodiment, the method comprises providing a plurality of small organic compounds, e.g., for halogenation.

The peroxidase may be any peroxidase known in nature, including a haloperoxidase. In an embodiment, the haloperoxidase is a vanadium haloperoxidase (VHPO). In an embodiment, the VHPO is a vanadium chloroperoxidase (VCPO), vanadium bromoperoxidase (VBPO), or vanadium iodoperoxidase (VHPO). In an embodiment, the VHPO is a VBPO. The peroxidase may be an algal haloperoxidase (e.g., derived from an algal species) or a fungal haloperoxidase (e.g., derived from a fungal species). In an embodiment, the peroxidase is a fungal haloperoxidase (e.g., derived from a fungal species). The peroxidase may be derived from an organism selected from Curvularia inaequalis, Halomicronema hongdechloris, Moorea bouillonii, Trichodesmium erythraeum, Aphanocapsa montana, Lyngbya confervoides, Synechococcus sp. PCC7335, and Corallina officinalis. In an embodiment, the peroxidase is derived from Corallina officinalis. In an embodiment, the peroxidase is derived from Aphanocapsa montana. In an embodiment, the peroxidase is derived from Curvularia inaequalis. In an embodiment, the peroxidase comprises a sequence of a peroxidase described herein, e.g., a peroxidase sequence provided in Table 2.

The peroxidase may be produced in a host cell microorganism, e.g., overexpressed in a host cell microorganism. In an embodiment, the host cell microorganism is selected from Pichia pastoris, Aspergillus niger, Saccharomyces cerevisiae, or Escherichia col. In an embodiment, expression of the peroxidase produced in the host cell microorganism is increased by about 1.5-fold, 2-fold, 3-fold, 4-fold, 5-fold, 6-fold, 7-fold, 8-fold, or 10-fold, e.g., over a peroxidase produced in its native host. In an embodiment, the amino acid sequence of the peroxidase is selected from an amino acid sequence listed in Table 2. In an embodiment, the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from the list in Table 2. In an embodiment, the peroxidase is a sequence selected from any one of SEQ ID NOs. 1-50. In an embodiment, the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from SEQ ID NOs: 1-50. In an embodiment, the peroxidase an amino acid sequence selected from any one of SEQ ID NOs. 1-50.

The small organic compound may be a naturally occurring or non-naturally occurring compound. For example, the small organic compound may comprise be a natural product, a lipid, a sterol, a steroid, an amino acid, a sugar, a phlorotannin, a tannin, a lignin, or a lignin derivative. In an embodiment, the small organic compound comprises a functional group, e.g., an aldehyde, ketone, acetyl, acyl, hydroxyl, ester, ether, amine, amide, aryl, heteroaryl, heterocyclyl, or cycloalkyl group. In an embodiment, the small organic compound comprises an alkenyl or alkynyl group. In an embodiment, the small organic compound comprises an aldehyde or ketone group. In an embodiment, the small organic compound comprises an alpha-beta unsaturated ketone. In an embodiment, the small organic compound is acetone or acetylacetone. In an embodiment, the small organic compound comprises 1, 2, 3, 4, 5, 6, 7, or 8 carbon atoms. In an embodiment, the small organic compound comprises a ketone or aldehyde.

In an embodiment, the small halogenated organic compound comprises a natural product, a lipid, a sterol, a steroid, an amino acid, a sugar, a phlorotannin, a tannin, a lignin, or a lignin derivative is chlorinated, brominated, or iodinated. In an embodiment, the small halogenated organic compound is brominated. In an embodiment, the small halogenated organic compound comprises a functional group, e.g., an aldehyde, ketone, acetyl, acyl, hydroxyl, ester, ether, amine, amide, aryl, heteroaryl, heterocyclyl, or cycloalkyl group. In an embodiment, the small halogenated organic compound comprises an alkenyl or alkynyl group. In an embodiment, the small halogenated organic compound comprises an aldehyde or ketone group. In an embodiment, the small halogenated organic compound comprises an alpha-beta unsaturated ketone. In an embodiment, the small halogenated organic compound comprises 1, 2, 3, 4, 5, 6, 7, or 8 carbon atoms. In an embodiment, the small halogenated organic compound comprises 1, 2, 3 halogen atoms. In an embodiment, the small halogenated organic compound comprises 1, 2, 3 bromine atoms. In an embodiment, the small halogenated organic compound comprises an acetone moiety. In an embodiment, the small halogenated organic compound comprises dibromoacetone, bromoacetone, bromopentanedione, bromoform, or tribromoacetone. In an embodiment, the small halogenated organic compound comprises 1,1-dibromoacetone, bromoacetone, 3-bromo-2,4-pentanedione, bromoform, 1,1,3-tribromoacetone, or 1,1,1-tribromoacetone. In an embodiment, the small halogenated compound comprises dichloroiodomethane, dichlorobromomethane, dibromoiodomethane, diiodochloromethane, or diiodobromomethane. In an embodiment, the small halogenated organic compound comprises 1,1-dibromoacetone, bromoacetone, 3-bromo-2,4-pentanedione, bromoform, 1,1,3-tribromoacetone, or 1,1,1-tribromoacetone, dichloroiodomethane, dichlorobromomethane, dibromoiodomethane, diiodochloromethane, or diiodobromomethane.

In one aspect, the small organic compound comprises a compound of Formula (Y):

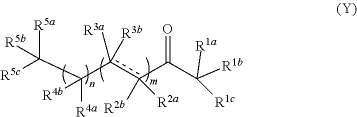

or a salt, tautomer, or isomer thereof, wherein each of R^{1a}, R^{1b}, R^{1c}, R^{2a}, R^{2b}, R^{3a}, R^{3b}, R^{4a}, R^{4b}, R^{5a}, R^{5b}, and R^{5c} is independently hydrogen, halogen, C₁-C₆ alkyl, C₁-C₆ heteroalkyl, C₂-C₆ alkenyl, C₂-C₆ alkynyl, cycloalkyl, or heterocyclyl, wherein each alkyl, heteroalkyl, alkenyl, alkynyl, cycloalkyl or heterocyclyl is optionally substituted with one or more R⁶; R⁶ is halogen, C₁-C₆ alkyl, C₁-C₆ heteroalkyl, C₂-C₆ alkenyl, C₂-C₆ alkynyl, —OR^{A}, or —NR^{B}R^{C}; R^{A} is hydrogen, C₁-C₆ alkyl, C₁-C₆ heteroalkyl, or C₂-C₆ alkenyl; R^{B} and R^{C} are each independently hydrogen, C₁-C₆ alkyl, or C₁-C₆ heteroalkyl; each of m and n is independently an integer between 0 and 24; and “ [symbol] ” is a single or double bond, wherein when [symbol] is a double bond, each of R^{2b} and R^{3b} is independently absent.

In an embodiment of Formula (Y), each of R^{1a}, R^{1b}, R^{1c}, R^{2a}, R^{2b}, R^{3a}, R^{3b}, R^{5a}, R^{5b}, and R^{5c} are each independently hydrogen. In an embodiment of Formula (Y), m is selected from 0, 1, 2, or 3. In an embodiment of Formula (Y), n is selected from 0, 1, 2, or 3. In an embodiment of Formula (Y), [symbol] is a single bond. In an embodiment of Formula (Y), each of R^{1a}, R^{1b}, R^{1c}, R^{2a}, R^{2b}, R^{3a}, R^{3b}, R^{5a}, R^{5b}, and R^{5c} are each independently hydrogen, each of m and n is independently selected from 0, 1, 2, or 3, and [symbol] is a single bond.

In an embodiment of Formula (Y), each of R^{1a}, R^{1b}, R^{1c}, R^{5a}, R^{5b}, and R^{5c} are each independently hydrogen, and each of m and n is 0. In an embodiment of Formula (Y), each of R^{1a}, R^{1b}, R^{1c}, R^{2a}, R^{2b}, R^{3a}, R^{3b}, R^{5a}, R^{5b}, and R^{5c} are each independently hydrogen, n is 0, m is 1, and [symbol] is a single bond. In an embodiment of Formula (Y), R^{1a} is C₁ alkyl; R^{1b}, R^{1c}, R^{5a}, R^{5b}, and R^{5c} are each independently hydrogen, and each of m and n is 0. In an embodiment of Formula (Y), R^{1a} is halogen (e.g., chlorine, bromine, or iodine); R^{1b}, R^{1c}, R^{5a}, R^{5b}, and R^{5c} are each independently hydrogen, and each of m and n is 0.

In another aspect, the small halogenated organic compound comprises a compound of Formula (Z):

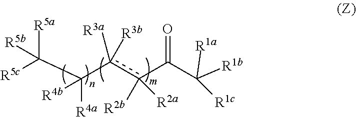

or a salt, tautomer, or isomer thereof, wherein each of R^{1a}, R^{1b}, R^{1c}, R^{2a}, R^{2b}, R^{3a}, R^{3b}, R^{4a}, R^{4b}, R^{5a}, R^{5b}, and R^{5c} is independently hydrogen, halogen, C₁-C₆ alkyl, C₁-C₆ heteroalkyl, C₂-C₆ alkenyl, C₂-C₆ alkynyl, cycloalkyl, or heterocyclyl, wherein each alkyl, heteroalkyl, alkenyl, alkynyl, cycloalkyl or heterocyclyl is optionally substituted with one or more R⁶, and at least one of R^{1a}, R^{1b}, R^{1c}, R^{2a}, R^{2b}, R^{3a}, R^{3b}, R^{4a}, R^{4b}, R^{5a}, R^{1b}, and R^{1c} is independently halogen; R⁶ is halogen, C₁-C₆ alkyl, C₁-C₆ heteroalkyl, C₂-C₆ alkenyl, C₂-C₆ alkynyl, —OR^{A}, or —NR^{B}R^{C}; R^{A} is hydrogen, C₁-C₆ alkyl, C₁-C₆ heteroalkyl, or C₂-C₆ alkenyl; R^{B} and R^{C} are each independently hydrogen, C₁-C₆ alkyl, or C₁-C₆ heteroalkyl; each of m and n is independently selected from 0, 1, 2, or 3; and “ [symbol] ” is a single or double bond, wherein when [symbol] is a double bond, each of R^{2b} and R^{3b} is independently absent.

In an embodiment of Formula (Z), each of R^{1a}, R^{1b}, and R^{1c} is independently halogen or hydrogen, wherein at least one of R^{1a}, R^{1b}, and R^{1c} is halogen. In an embodiment, the halogen is selected from chlorine, bromine, or iodine. In an embodiment of Formula (Z), each of R^{1a}, R^{1b}, R^{1c} is independently halogen or hydrogen, wherein at least two of R^{1a}, R^{1b}, and R^{1c} is halogen. In an embodiment, the halogen is selected from two of chlorine, bromine, or iodine. In an embodiment of Formula (Z), each of R^{1a}, R^{1b}, R^{1c} is independently halogen. In an embodiment, the halogen is selected from chlorine, bromine, or iodine. In an embodiment of Formula (Z), each of R^{5a}, R^{5b}, and R^{5c} is independently halogen or hydrogen, wherein at least one of R^{5a}, R^{5b}, and R^{5c} is halogen. In an embodiment of Formula (Z), each of R^{5a}, R^{5b}, and R^{5c} is independently halogen or hydrogen, wherein at least two of R^{5a}, R^{5b}, and R^{5c} is halogen. In an embodiment of Formula (Z), each of R^{5a}, R^{5b}, and R^{5c} is independently halogen. In an embodiment of Formula (Z), [symbol] is a single bond. In an embodiment of Formula (Z), each of m and n is independently selected from 0, 1, 2, or 3, and [symbol] s a single bond.

In an embodiment, the conditions sufficient to produce a small halogenated organic compound comprise one or more of: (a) temperature between 10° C. to 85° C.; (b) pH between 4-10; and (c) an ionic strength between 0.1 mM to 4 M. In an embodiment, the conditions sufficient to produce a small halogenated organic compound comprise a temperature between 10° C. to 85° C. In an embodiment, the conditions sufficient to product a small halogenated organic compound comprise a pH between 4 and 10. In an embodiment, the conditions sufficient to 5 product a small halogenated organic compound comprise an ionic strength between 0.1 mM to 4 M. In an embodiment, the evaluating comprises analysis of the small halogenated organic compound by an analytical technique. In an embodiment, the analytical technique comprises HPLC, GC-MS, NMR.

The small halogenated organic compound may be useful for a number of agricultural, marine, and/or industrial processes. In an embodiment, the small halogenated organic compound is capable of reducing methane production by a microbial organism. In an embodiment, methane production is reduced by at least 5%, 10%, 15%, 20%, 25% 30%, 35%, 40%, 50%, 55%, 60%, 65%, 70%, 75%, 80%, 85%, 90%, 95%, 99%, or more. In an embodiment, the methane production is reduced by between 10-75%. In an embodiment, the microbial organism is present within a rumen community.

In another aspect, the present disclosure features a method for reducing production of a small hydrocarbon (e.g., methane) in a rumen community, comprising: (i) providing a small organic compound (e.g., acetyl acetone); (ii) contacting the small organic compound with a peroxidase (e.g., a VHPO) to form a reaction mixture under conditions sufficient to produce a small halogenated organic compound; (iii) separating the small halogenated organic compound from the reaction mixture; and/or (iv) providing the small halogenated organic compound to a rumen community under conditions sufficient to reduce the production of a small hydrocarbon (e.g., methane). In an embodiment, the method features (i). In an embodiment the method features (ii). In an embodiment, the method features (iii). In an embodiment, the method features (iv). In an embodiment, the method features (i) and (ii). In an embodiment, the method features (i) and (iii). In an embodiment, the method features (i) and (iv). In an embodiment, the method features (ii) and (iii). In an embodiment, the method features (ii) and (iv). In an embodiment, the method features (iii) and (iv). In an embodiment, the method features each of (i)-(iv).

In an embodiment, the method features (iii) separating the small halogenated organic compound from the reaction mixture, and (iv-a) incorporating the separated small halogenated organic compound into a matrix for delivery to the rumen community under conditions sufficient to reduce the production of a small hydrocarbon (e.g., methane). In an embodiment the method features (iv-a) incorporating the separated small halogenated organic compound into a matrix for delivery to the rumen community under conditions sufficient to reduce the production of a small hydrocarbon (e.g., methane). In an embodiment the method features each of (i), (ii), (iii) and (iv-a). In an embodiment the method features (ii) and (iv-a). In an embodiment the method features each of (i), (ii), and (iv-a). In an embodiment the method features (iii) and (iv-a). In an embodiment the method features each (i), (iii) and (iv-a). In an embodiment the method features each of (ii), (iii), and (iv-a).

In an embodiment, the method further comprises acquiring a value for the level of a small hydrocarbon (e.g., methane) (a) prior to providing the peroxidase or (b) after providing the peroxidase. In an embodiment, the method further comprises (a). In an embodiment, the method further comprises (b). In an embodiment, the method further comprises (a) and (b). In an embodiment, methane production is reduced by at least 5%, 10% 0.15%, 20%, 25% 30%, 35%, 40%, 50%, 55%, 60%, 65%, 70%, 75%, 80%, 85%, 90%, 95%, 99%, or more. In an embodiment, the methane production is reduced by between 10-75%.

The peroxidase may be any peroxidase known in nature, including a haloperoxidase. In an embodiment, the haloperoxidase is a vanadium haloperoxidase (VHPO). In an embodiment, the VHPO is a vanadium chloroperoxidase (VCPO), vanadium bromoperoxidase (VBPO), or vanadium iodoperoxidase (VHPO). In an embodiment, the VHPO is a VBPO. The peroxidase may be an algal haloperoxidase (e.g., derived from an algal species) or a fungal haloperoxidase (e.g., derived from a fungal species). In an embodiment, the peroxidase is a fungal haloperoxidase (e.g., derived from a fungal species). The peroxidase may be derived from an organism selected from Curvularia inaequalis, Halomicronema hongdechloris, Moorea bouillonii, Trichodesmium erythraeum, Aphanocapsa montana, Lyngbya confervoides, Synechococcus sp. PCC7335, and Corallina officinalis. In an embodiment, the peroxidase is derived from Corallina officinalis. In an embodiment, the peroxidase is derived from Aphanocapsa montana. In an embodiment, the peroxidase is derived from Curvularia inaequalis.

The peroxidase may be produced in a host cell microorganism, e.g., overexpressed in a host cell microorganism. In an embodiment, the host cell microorganism is selected from Pichia pastoris, Aspergillus niger, or Escherichia coli. In an embodiment, expression of the peroxidase produced in the host cell microorganism is increased by about 1.5-fold, 2-fold, 3-fold, 4-fold, 5-fold, 6-fold, 7-fold, 8-fold, or 10-fold, e.g., over a peroxidase produced in its native host. In an embodiment, the amino acid sequence of the peroxidase is selected from an amino acid sequence listed in Table 2. In an embodiment, the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from the list in Table 2. In an embodiment, the peroxidase is a sequence selected from any one of SEQ ID NOs. 1-50. In an embodiment, the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from SEQ ID NOs: 1-50. In an embodiment, the amino acid sequence of the peroxidase has at 1, 2, 3, 4, 5, or 6 amino acid substitutions relative to an amino acid sequence selected from any one of SEQ ID NOs. 1-50.

In an embodiment, the method features: (i) providing a small organic compound comprising an alpha-beta unsaturated ketone (e.g., acetyl acetone; and (ii) contacting the small organic compound with a peroxidase (e.g., a VHPO) to form a reaction mixture under conditions sufficient to produce a small halogenated organic compound. In an embodiment, the method further comprises, for step (ii), providing a halogen source and a peroxide source. In an embodiment, the halogen source provides a halogen anion (e.g., F—, Cl—, Br—, and/or I—). In an embodiment, the peroxide source is hydrogen peroxide. In an embodiment, the method further comprises, for step (ii), providing a halogen source and a peroxide source in the presence of an amine, e.g., to produce a chlorinated amine, e.g. NH₂Cl. In an embodiment, the providing a halogen course comprises providing a chlorinated amine (e.g. NH₂Cl).

In an embodiment, the further comprises, for step (ii), providing a peroxide source and a halogen source (e.g., a bromine source, a chloride source, or an iodine source) to the peroxidase, e.g., in the presence of an amine to generate a halogenated amine, e.g. NH₂Cl. In an embodiment, the method further comprises, for step (ii), reacting the halogenated amine with an additional halogen source to further halogenate the small halogenated organic compound. In an embodiment, the additional halogen source is a bromide anion, a chlorine anion, or an iodide anion. In an embodiment, the reacting the halogenated amine with an additional halogen source occurs in a separate vessel from step (i) or (ii).

In an embodiment, the method features: (ii) providing a peroxide source and a chloride source to a peroxidase (e.g., a VCPO) in the presence of an amine to generate a chlorinated amine, e.g. NH₂Cl; and (iii) reacting the chlorinated amine with two equivalents of iodide anion (I⁻) to generate a mixture of hypoiodite anion and iodine (I₂). In an embodiment, (ii) occurs in a separate reaction vessel from (iii). In an embodiment, the method further comprises (iv) contacting the mixture of hypoiodite anion and iodine from (iii) with the small halogenated compound, e.g., produced in (i) to generate an additional small halogenated organic compound (e.g., a further halogenated small halogenated organic compound, e.g., dichloroiodomethane or dibromoiodomethane). In an embodiment, the method features (i). In an embodiment, the method features (ii) and (iii). In an embodiment, the method features (i), (ii), (iii) and (iv). In an embodiment, the method features (ii) and (iv). In an embodiment, the method features (ii), (iii) and (iv). In all embodiments, (ii) can be substituted by (iib).

In an embodiment, the yield from step (i) is between about 50% and 99%. In an embodiment, the yield from step (i) is between about 75% and 99. In an embodiment, the yield from step (i) is between about 85% and 99%. In an embodiment, the yield of steps (i), (ii), (iii) and (iv) is between about 10% and 99%. In an embodiment, the yield of steps (i), (ii), (iii) and (iv) is between about 10% and 75%. %. In an embodiment, the yield of steps (i), (ii), (iii) and (iv) is between about 10% and 50%. In an embodiment, the yield of steps (i), (ii), (iii) and (iv) is between about 25% and 99%. In an embodiment, the method is carried out at a pH between about 5.0 and 9.0. In an embodiment, steps (ii) and/or (ii) are carried out at a pH between 7.0 and 8.0, e.g., a pH between 7.2 and 7.8, a pH between 7.5 and 8.0. Without being bound by theory, keeping the pH of steps (ii) and/or (iii) between a pH between about 7.0 and 8.0 may aid in reducing side reactions generating I₃⁻ or iodate anion IO₃⁻.

In an embodiment, the method comprising the enzymatic reaction of organic peroxide, e.g. peracetic acid (PAA), and a peroxidase (e.g., VCPO) features a reduction between about 10%-99% in the conversion rate of hypohalite anion and excess iodide anion to diatomic iodine I₂ and triiodide anion I₃⁻. In an embodiment the method comprising the enzymatic reaction of organic peroxide, e.g., PAA, and a peroxidase (e.g., VCPO) features a reduction between 10%-99% in the conversion rate of hypohalite anion and excess iodide anion to diatomic iodine I₂ and triiodide anion I₃⁻ at pH 7 over the method comprising the enzymatic reaction of H₂O₂ and VHPO at pH 7. In an embodiment the method comprising the enzymatic reaction of organic peroxide, e.g., PAA, and a peroxidase (e.g., VCPO) features a reduction between 10%-99% in the conversion rate of hypohalite anion and bromide anion to diatomic bromine Br₂ at pH between 0-5 over the method comprising the enzymatic reaction of H₂O₂ and a VHPO at pH between 0-5.

In an embodiment the method comprising the enzymatic reaction of an organic peroxide (e.g., PAA) and a peroxidase (e.g., VCPO) features a reduction between 10%-99% in the conversion rate of hypohalite anion and chloride anion to diatomic chlorine Cl₂ at pH between 0-5 over the method comprising the enzymatic reaction of H₂O₂ and a VHPO at pH between 0-5. In an embodiment the method comprising the enzymatic reaction of organic peroxide, e.g. PAA, and a peroxidase (e.g., VCPO) features a reduction between 10%-99% in the conversion rate of peracetic acid and bromide anion to hypobromite anion or its conjugate acid at pH between 0-5 over the method comprising the enzymatic reaction of H₂O₂ and VHPO at pH between 0-5. In an embodiment the method comprising the enzymatic reaction of organic peroxide, e.g. PAA, and a peroxidase (e.g., VCPO) features a reduction between 10%-99% in the conversion rate of peracetic acid and iodide anion to hypoiodite anion or its conjugate acid at pH between 0-5 over the method comprising the enzymatic reaction of organic peroxide, e.g. PAA, and VHPO at pH between 0-5.

In an embodiment the method comprising the enzymatic reaction of organic peroxide, e.g. PAA, and a peroxidase (e.g., VCPO) (e.g. step (ii)) features an increase between 10%-500% in the k_{cat} over the method comprising the enzymatic reaction of H₂O₂ and a VHPO. In an embodiment the method comprising the enzymatic reaction of organic peroxide, e.g. PAA, and a peroxidase (e.g., VCPO) features an increase between 25%-250% in the k_{cat} over the method comprising the enzymatic reaction of H₂O₂ and a VHPO. In an embodiment the method comprising the enzymatic reaction of organic peroxide, e.g. PAA, and a peroxidase (e.g., VCPO) features an increase between 50%-150% in the k_{cat} over the method comprising the enzymatic reaction of H₂O₂ and a VHPO. In an embodiment the method comprising the enzymatic reaction of organic peroxide, e.g. PAA, and VCPO features an increase between 75%-125% in the kat over the method comprising the enzymatic reaction of H₂O₂ and a VHPO. In an embodiment the method comprising the enzymatic reaction of organic peroxide, e.g. PAA, and a peroxidase (e.g., VCPO) features an increase between 80%-120% in the k_{cat} over the method comprising the enzymatic reaction of H₂O₂ and a VHPO. In an embodiment the method comprising the enzymatic reaction of organic peroxide, e.g. PAA, and VCPO features an increase between 90%-110% in the k_{cat} over the method comprising the enzymatic reaction of H₂O₂ and a VHPO.

In an embodiment the method comprising the enzymatic reaction of organic peroxide, e.g. PAA, and a peroxidase (e.g., VCPO) (e.g. step (ii)) features an increase between 10%-500% in the k_{cat} over the method comprising the enzymatic reaction of H₂O₂ and a VHPO, wherein dichloriodomethane is generated. In an embodiment the method comprising the enzymatic reaction of organic peroxide, e.g. PAA, and a peroxidase (e.g., VCPO) features an increase between 25%-250% in the k_{cat} over the method comprising the enzymatic reaction of H₂O₂ and a VHPO, wherein dichloriodomethane is generated. In an embodiment the method comprising the enzymatic reaction of organic peroxide, e.g. PAA, and a peroxidase (e.g., VCPO) features an increase between 50%-150% in the k_{cat} over the method comprising the enzymatic reaction of H₂O₂ and a VHPO, wherein dichloriodomethane is generated. In an embodiment the method comprising the enzymatic reaction of organic peroxide, e.g. PAA, and a peroxidase (e.g., VCPO) features an increase between 75%-125% in the k_{cat} over the method comprising the enzymatic reaction of H₂O₂ and a VHPO, wherein dichloriodomethane is generated. In an embodiment the method comprising the enzymatic reaction of organic peroxide, e.g. PAA, and a peroxidase (e.g., VCPO) features an increase between 80%-120% in the k_{cat} over the method comprising the enzymatic reaction of H₂O₂ and a VHPO, wherein dichloriodomethane is generated. In an embodiment the method comprising the enzymatic reaction of organic peroxide, e.g. PAA, and a peroxidase (e.g., VCPO) features an increase between 90%-110% in the k_{cat} over the method comprising the enzymatic reaction of H₂O₂ and a VHPO, wherein dichioriodomethane is generated. In an embodiment the method comprising the enzymatic reaction of organic peroxide, e.g. PAA, and a peroxidase (e.g., VCPO) features an increase between 10%-500% in the k_{cat} over the method comprising the enzymatic reaction of H₂O₂ and a VHPO, wherein dibromochloromethane is generated. In an embodiment the method comprising the enzymatic reaction of organic peroxide, e.g. PAA, and a peroxidase (e.g., VCPO) features an increase between 25%-250% in the k_{cat} over the method comprising the enzymatic reaction of H₂O₂ and a VHPO, wherein dibromoiodomethane is generated. In an embodiment the method comprising the enzymatic reaction of organic peroxide, e.g. PAA, and and a peroxidase (e.g., VCPO) features an increase between 50%-150% in the k_{cat} over the method comprising the enzymatic reaction of H₂O₂ and a VHPO, wherein dibromoiodomethane is generated. In an embodiment the method comprising the enzymatic reaction of organic peroxide, e.g. PAA, and a peroxidase (e.g., VCPO) features an increase between 75%-125% in the k_{cat} over the method comprising the enzymatic reaction of H₂O₂ and a VHPO, wherein dibromoiodomethane is generated. In an embodiment the method comprising the enzymatic reaction of organic peroxide, e.g. PAA, and a peroxidase (e.g., VCPO) features an increase between 80%-120% in the k_{cat} over the method comprising the enzymatic reaction of H₂O₂ and a VHPO, wherein dibromoiodomethane is generated. In an embodiment the method comprising the enzymatic reaction of organic peroxide, e.g. PAA, and a peroxidase (e.g., VCPO) features an increase between 90%-110% in the k_{cat} over the method comprising the enzymatic reaction of H₂O₂ and a VHPO, wherein dibromoiodomethane is generated.

The small organic compound may be a naturally occurring or non-naturally occurring compound. In an embodiment, the small organic compound comprises 1, 2, 3, 4, 5, 6, 7, or 8 carbon atoms. In an embodiment, the small organic compound comprises a ketone or aldehyde. In an embodiment, the small halogenated organic compound is chlorinated, brominated, or iodinated. In an embodiment, the small halogenated organic compound is brominated. In an embodiment, the small halogenated organic compound comprises 1, 2, 3 halogen atoms. In an embodiment, the small halogenated organic compound comprises 1, 2, 3 bromine atoms. In an embodiment, the small halogenated organic compound comprises an acetone moiety. In an embodiment, the small halogenated organic compound comprises dibromoacetone, bromoacetone, bromopentanedione, bromoform, or tribromoacetone. In an embodiment, the small halogenated organic compound comprises 1,1-dibromoacetone, bromoacetone, 3-bromo-2,4-pentanedione, bromoform, 1,1,3-tribromoacetone, or 1,1,1-tribromoacetone.

In an embodiment, the conditions sufficient to product a small halogenated organic compound comprise one or more of: (a) temperature between 10° C. to 85° C.; (b) pH between 4-10; and (c) an ionic strength between 0.1 mM to 4 M. In an embodiment, the conditions sufficient to product a small halogenated organic compound comprise a temperature between 10° C. to 85° C. In an embodiment, the conditions sufficient to product a small halogenated organic compound comprise a pH between 4-10. In an embodiment, the conditions sufficient to product a small halogenated organic compound comprise an ionic strength between 0.1 mM and 4 M. In an embodiment, the evaluating comprises analysis of the small halogenated organic compound by an analytical technique. In an embodiment, the analytical technique comprises HPLC, GC-MS, NMR.

The small halogenated organic compound may be useful for a number of agricultural, marine, and/or industrial processes. In an embodiment, the small halogenated organic compound is capable of reducing methane production by a microbial organism. In an embodiment, methane production is reduced by at least 5%, 10% 0.15%, 20%, 25% 30%, 35%, 40%, 50%, 55%, 60%, 65%, 70%, 75%, 80%, 85%, 90%, 95%, 99%, or more. In an embodiment, the methane production is reduced by between 10-75%. In an embodiment, the microbial organism is present within a rumen community.

In another aspect, the method features a method for increasing expression of a peroxidase in a host cell or host microorganism.

In another aspect, the method further features a reactor for the continuous production of a small halogenated organic compound comprising: (i) a reaction chamber; (ii) a module for temperature control; (iii) a peristaltic pump; and/or (iv) a module for housing a catalyst.

Additional embodiments of the present disclosure are described in further detail herein in the Drawings, Description, Examples, and Claims.

### BRIEF DESCRIPTION OF DRAWINGS

FIG. 1 is an example of the chemical reactions catalyzed by vanadium haloperoxidases.

FIGS. 2A-2C are enzymatic reaction mechanisms catalyzed by vanadium haloperoxidases. FIG. 2A is an equation describing a general ping-pong bi-bi reaction mechanism. FIG. 2B is a Cleland equation for the ping-pong bi-bi reaction with substrate inhibition. FIG. 2C is an equation describing reaction parameters of the vanadium haloperoxidases formulated through the King-Altman method.

FIGS. 3A-3C show the influence of pH and ionic strength on exemplary CoVBPO kinetics. FIG. 3A illustrates the pH dependence of K_{mBr-} for high (2.0 M) and low (0.2 M) concentrations of MgSO₄. Further details of the kinetics are given in Tables 3 and 4. FIG. 3B illustrates the relationship between k_{cat} and the deuterium isotope fraction for each of the ionic strength solutions; and FIG. 3C illustrates the solvent isotope effect (SIE) on k_{cat} and K_{mBr-}. This information was used to interpret the reaction mechanism and reduce the K_{mBr-} 12-fold (K_{mBr}=0.8 mM.

FIGS. 4A-4C show the CiVCPO-driven production of small halogenated organic compounds from the exemplary small organic compound acetylacetone. FIG. 4A shows the concentration of reaction products over time in an exemplary CiVCPO reaction. FIG. 4B is an HPLC-derived chromatogram of the reaction products from the CiVCPO reaction, illustrating how 1,1, dibromoacetone was fractionated for use in methane-suppression experiments. FIG. 4C is a GC-MS chromatogram showing the components of the n-hexane extract of the HPLC-fractionated 1,1-dibromoacetone. The identity of the main product was made by comparison of the product MS spectrum with a 1,1-dibromoacetone standard.

FIG. 5 is an illustration of the exemplary workflow used for generating and testing the methane-suppression potential of small halogenated organic compounds.

FIGS. 6A-6C are graphs demonstrating the influence of small halogenated compounds on methane production by a rumen-derived microbial culture. The rates of methane production are shown for additions of 1,1-dibromoacetone, bromoform, and dibromomethane.

FIG. 7 is a sequence alignment of several exemplary VHPOs.

FIG. 8 is an SDS-PAGE polyacrylamide gel after purification of cchVBPO from heterologous production in Escherichia coli.

FIG. 9 is an SDS-PAGE polyacrylamide gel after purification of synVBPO from heterologous production in Escherichia coli.

FIGS. 10A-B shows the reaction kinetics of synVBPO and fits of the kinetic parameters to Michael-Menten and Hill-type kinetics. FIG. 10A is a surface plot of synVBPO halogenating activity showing k_{cat} as a function of H₂O₂ and KBr concentration. FIG. 10B describes the fitting of kinetic parameters to halogenation reaction velocity employing Michaelis-Menten and Hill-type kinetics. Akaike's Information Criterion Test (AIC), Bayesian Information Criterion Test (BIC), and associated statistics are also shown to determine best fit of the candidate models.

FIGS. 11A-D are Salwin tests depicting enzyme stability of synVBPO at various concentrations of KBr and H₂O₂. FIG. 11A is a Salwin Test of syn VBPO at 160 mM KBr and 50 μM H₂O₂. FIG. 11B is a Salwin Test of synVBPO at 40 mM KBr and 50 μM H₂O₂. FIG. 11C is a Salwin Test of synVBPO at 1 mM KBr and 50 μM H₂O₂. FIG. 11D is a Salwin Test of synVBPO at 1 mM KBr and 250 μM H₂O₂.

FIGS. 12A-D illustrate the enzymatic activity of synVBPO as a function of ionic strength and the determination of Michaelis-Menten kinetic parameters as a function of pH. FIG. 12A is a plot of change in fluorescence per second as a function of ionic strength of VBPO reaction velocity in the presence of peroxide and halide substrate. FIG. 12B is a plot of synVBPO k_{cat} as function of pH. FIG. 12C is a plot of synVBPO k_{cat}/Km_{H2O2} as function of pH. FIG. 12D is a plot of synVBPO k_{cat}/Km_{KBr} as function of pH.

FIG. 13 is a plot of the VCPO reaction velocity in the presence of the peroxide sources H₂O₂ and peracetic acid (PAA) and a calculation of the Michaelis-Menten kinetic parameters, demonstrating that the V_{max, PAA} is approximately twice that of V_{max, H2O2}.

FIG. 14 is a graph of the reaction velocity of a VCPO with a Michaelis Menten curve fit equation with PAA and H₂O₂ as substrates, demonstrating that PAA has both a higher vₘₐₓ and Kₘ than hydrogen peroxide.

FIG. 15 is a graph of DCIM concentration produced versus time in a two-step fed-batch system.

FIG. 16 is an illustration of the dissolved oxygen cascade in the glycerol batch phase.

FIG. 17 is an SDS-PAGE polyacrylamide gel showing the result of the purification protocols outlined in the Examples.

### DETAILED DESCRIPTION

The present disclosure features peroxidases (e.g., haloperoxidases) and related compositions thereof for use in producing a small halogenated organic compound or a plurality of small halogenated organic compounds. These small halogenated organic compounds may be useful for reducing methane production in a microorganism or rumen community. In addition, the present disclosure features methods for producing peroxidases in a robust and efficient manner, as well as reactors and other devices for housing and monitoring peroxidase activity.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

“About”, when used herein to modify a numerically defined parameter means that the parameter may vary by as much as 15% above or below the stated numerical value for that parameter. For example, a small organic compound defined as having a molecular weight of 100 Da may have a molecular weight of between 85 Da to 115 Da. In some embodiments, about means that the parameter may vary by as much as 10% above or below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometry to acquire molecular weight information.

A “halogenation agent,” as that term is used herein, refers to an agent (e.g., a small molecule or a protein) capable of modifying an entity with a halogen, for example, with a fluorine, chlorine, bromine, or iodine atom. In an embodiment, the halogenation agent is a small molecule or salt, such as potassium bromide. In another embodiment, the halogenation agent is a protein, such as a halogenase or a haloperoxidase (e.g., a vanadium haloperoxidase).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof. A “plurality of polypeptides” refers to two or more polypeptides, e.g., 2, 3, 4, 5, 6, 7, 8, 9, 10, 20, 50, 100, 200, or 500 or more polypeptides.

The term “peroxidase”, as used herein, refers to an enzyme that reduces peroxide-containing substrates or hydroperoxidase-containing substrates. Many peroxidases contain a cofactor, e.g., a heme cofactor, orthovanadate, or a redox-active side chain such as cysteine or selenocysteine. Exemplary peroxidases include haloperoxidases, ascorbate peroxidases, lactoperoxidases, thyroid peroxidases, and others.

The term “rumen” refers to a specialized enteric compartment found within certain animals, e.g. a ruminant animal, which carries out several digestive functions within the animal, e.g. fermentative processes.

The term “ruminant animal” refers to an animal with a specialized enteric compartment which carries out several digestive functions within the animal, e.g. fermentative processes.

The terms “rumen community,” or “rumen microbial community” refers to a population of microorganisms including bacteria, archaea, and protozoa that populate the digestive tract of a large animal, e.g., an ruminant animal. The rumen microbial community carries out several digestive functions within the animal, including assisting in digestion to provide key nutrition to the host animal. Exemplary organisms that make up the microbial community include archaeal methanogens, examples include, Methanobrevibacter, Methanosarcina, and Methanocorpulusum. Exemplary fermentative bacterial genera found within the rumen include Coriobacteriaceae, Fibrobacter, Ruminococcus, Butyivibrio, Streptococcus, Prevotella, Succinimonas, Selenomonas, Lachnospira and Succinivibrio.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₅ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 12 carbon atoms (“C₂-C₁₂ alkenyl”), 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 12 carbon atoms (“C₂-C₁₀alkynyl”), 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, and heterocyclyl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

### Haloperoxidases

Vanadium haloperoxidases (VHPO) (EC 1.11.1.18) are a member of the oxidoreductase enzyme subfamily that contains vanadate as a prosthetic group. VHPOs catalyze the two-electron oxidation of halide ions in a substrate inhibited bi-bi ping-pong mechanism to the corresponding hypohalous acids, often without substrate specificity and regioselectivity. VHPOs are often found in brown, red, and green marine algae and are classified by the most electronegative halide they oxidize. For example, vanadium chloroperoxidases (VCPOs) oxidize chloride, bromide, and iodide; vanadium bromoperoxidases (VBPOs) oxidize bromide and iodide; and vanadium iodoperoxidases (VHPOs) oxidize iodide. VHPOs are capable of halogenating a broad range of organic compounds having both commercial and pharmaceutical interest, and exhibit stability in a wide range of conditions, including high temperatures, acidic and basic pH, oxidative conditions, and organic solvents. These features make VHPOs attractive candidates for use in various industrial transformations.

Among the most studied VHPOs are a class of VBPOs found in a family of seaweeds in the genus Corallina. Corallina officinalis is a calcareous red seaweed commonly found in tide pools and in sea water habitats. The VBPO from Corallina officinalis (CoVBPO) has been successfully isolated from the seaweed and studied both biochemically and structurally. CoVBPO is characterized by a large dodecameric (12×64 kDa) structure comprising twelve identical subunits. CoVBPO was shown to exhibit functional stability over a pH range of 5-10 and at temperatures up to 90° C.; in addition, it exhibits tolerance towards organic solvents such as ethanol, methanol, and propan-1-ol, making it an attractive enzyme for commercial biocatalysis. However, isolating suitable quantities of CoVBPO from Corallina officinalis directly for industrial use has been challenging.

Industrial enzymes are enzymes that are commercially used in a variety of industries such as pharmaceuticals, chemical production, biofuels, food & beverage, and consumer products. Due to advancements in heterologous expression in hosts, like Escherichia coli and various fungi, biocatalysis through isolated enzymes may be more economical than using whole cells. However, in many cases, the heterologous expression of foreign proteins in E. coli results in the production of insoluble inclusion bodies because of protein misfolding. In particular, heterologous expression of large quantities of pure CoVBPO from E. coli has proven elusive, due to its tendency to form large insoluble inclusion bodies. As such, analogs of CoVBPO were sought from publicly available databases that may exhibit improved properties.

In an embodiment, the peroxidase (e.g., haloperoxidase) comprises an amino acid sequence of Formula (I): TGPX₁, wherein X₁ is proline or threonine. In an embodiment, the amino acid sequence of Formula (I) is selected from TGPP and TGPT. In an embodiment, the peroxidase (e.g., haloperoxidase) comprises an amino acid sequence of Formula (I-a): GPX₂TGPX₁, wherein X₁ is proline or threonine and X₂ is glutamine or leucine. In an embodiment, the amino acid sequence of Formula (I-a) is selected from GPPTGPQ, GPTTGPQ, GPPTGPL, and GPTTGPL. In an embodiment, the peroxidase (e.g., haloperoxidase) comprises an amino acid sequence of Formula (I-a): GPQTGPX₁ or GPLTGPX₁, wherein X₁ is proline or threonine.

In an embodiment, the peroxidase (e.g. haloperoxidase) comprises an amino acid sequence of Formula (II): X₃WKE, wherein X₃ is serine or alanine. In an embodiment, the amino acid sequence of Formula (II) is selected from SWKE and AWKE. In an embodiment, the peroxidase (e.g. haloperoxidase) comprises an amino acid sequence of Formula (II-a): GX₄FX₃WKE, wherein X₃ is serine or alanine and X₄ is isoleucine or valine. In an embodiment, the amino acid sequence of Formula (II-a) is selected from GIFSWKE, GIFAWKE, GVFSWKE and GVFAWKE. In an embodiment, the peroxidase (e.g. haloperoxidase) comprises an amino acid sequence of Formula (II-b): GIX₅X₃WX₆ wherein X₃ is serine or alanine; X₅ is phenylalanine or leucine; and X₆ is lysine, glutamate or glutamine. In an embodiment the amino acid sequence of Formula (II-b) is selected from GIFSWK, GIFSWE, GIFSWQ, GIFAWK, GIFAWE, GIFAWQ, GILSWK, GILSWE, GILSWQ, GIL,AWK, GILAWE, and GILAWQ.

In an embodiment, the peroxidase (e.g. haloperoxidase) comprises an amino acid sequence of Formula (III): X₇KW or X₇KY wherein X₇ is glutamate, or glutamine. In an embodiment the amino acid sequence of Formula (III) is selected from QKW and EKW. In an embodiment, the peroxidase (e.g. haloperoxidase) comprises an amino acid sequence of Formula (III-a): X₇KWX₈FE, wherein X₇ is glutamate or glutamine; and X₈ is glutamate, arginine, or histidine. In an embodiment, the amino acid sequence of Formula (III-a) is selected from EKWEFE, EKWRFE, EKWHFE, QKWEFE, QKWRFE, and QKWHFE. In an embodiment, the peroxidase (e.g. haloperoxidase) comprises an amino acid sequence of Formula (III-b): X₇KWX₈FEFW or X₇KYX₈FEFW, wherein X₇ is glutamate or glutamine; and X₈ is glutamate, arginine, or histidine. In an embodiment, the amino acid sequence of Formula (III-b) is selected from EKWEFEFW, EKWRFEFW, EKWHFEFW, QKWEFEFW, QKWRFEFW, QKWHFEFW, EKYEFEFW, EKYRFEFW, EKYHFEFW, QKYEFEFW, QKYRFEFW, and QKYHFEFW.

In an embodiment, the peroxidase (e.g. haloperoxidase) comprises an amino acid sequence of Formula (IV): YHX₉, wherein X₉ is glycine or alanine. In an embodiment, the amino acid sequence of Formula (IV) is selected from YHG and YHA. In an embodiment, the peroxidase (e.g. haloperoxidase) comprises an amino acid sequence of Formula (IV-a): YHX₉PFY, wherein X₉ is glycine or alanine. In an embodiment, the amino acid sequence of Formula (IV-a) is selected from YHGPFY and YHAPFY. In an embodiment, the peroxidase (e.g. haloperoxidase) comprises an amino acid sequence of Formula (IV-b): X₁₀YHGPFY or X₁₀YHAPFY, wherein Xio is glutamine, glycine, methionine, isoleucine. In an embodiment, the amino acid sequence of Formula (IV-b) is selected from QYHGPFY, GYHGPFY, MYHGPFY, IYHGPFY, QYHAPFY, GYHAPFY, MYHAPFY, and IYHAPFY. In an embodiment, the peroxidase (e.g. haloperoxidase) comprises an amino acid sequence of Formula (IV-c): FRX₁₁YHX₉PFY, wherein X₁₁ is glutamine, glycine, methionine, or isoleucine; and X₉ is glycine or alanine. In an embodiment, the amino acid sequence of Formula (IV-c) is selected from FRQYHGPFY, FRGYHGPFY, FRMYHGPFY, FRIYHGPFY, FRQYHAPFY, FRGYHAPFY, FRMYHAPFY, and FRIYHAPFY.

In an embodiment, the peroxidase (e.g. haloperoxidase) comprises an amino acid sequence of Formula (V): GVHX₁₂, wherein X₁₂ is tryptophan or tyrosine. In an embodiment, the amino acid sequence of Formula (V) is selected from GVHW and GVHF. In an embodiment, the peroxidase (e.g. haloperoxidase) comprises an amino acid sequence of Formula (V-a): GVHWX₁₃F, wherein X₁₃ is arginine, histidine, valine or glutamate. In an embodiment, the amino acid sequence of Formula (V-a) is selected from GVHWRF GVHWHF, GVHWVF, and GVHWEF. In an embodiment, the peroxidase (e.g. haloperoxidase) comprises an amino acid sequence of Formula (V-b): WX₁₃FDAX₁₄, wherein X₁₃ is arginine, histidine, valine or glutamate; and X₁₄ is alanine or phenylalanine. In an embodiment, the amino acid sequence of Formula (V-b) is selected from WRFDAA, WHFDAA, WVFDAA, WEFDAA, WRFDAF, WHFDAF, WVFDAF, and WEFDAF.

In an embodiment, the peroxidase (e.g. haloperoxidase) comprises an amino acid sequence of Formula (VI): X₁₅LI, wherein X₁₅ is asparagine or lysine. In an embodiment, the amino acid sequence of Formula (VI) is selected from NLI and KL. In an embodiment, the peroxidase (e.g. haloperoxidase) comprises an amino acid sequence of Formula (VI-a): DGX₁₆X₁₅LI, wherein X₁₅ is asparagine or lysine, and X₁₆ is serine or alanine. In an embodiment, the amino acid sequence of Formula (VI-a) is selected from DGSNLI, DGANLI, DGSKLI, and DGAKLI. In an embodiment, the peroxidase (e.g. haloperoxidase) comprises an amino acid sequence of Formula (VI-b): WX₁₇YDGX₁₆X₁₅LI, wherein X₁₅ is asparagine or lysine, X₁₆ is serine or alanine, and X, is alanine or glycine. In an embodiment, the amino acid sequence of Formula (VI-b) is selected from WGYDGSNLI, WAYDGSNLI, WAYDGANLI, WGYDGANLI, WGYDGSKLI, WAYDGSKLI, WAYDGAKLI, and WGYDGAKLI.

In an embodiment, the peroxidase (e.g. haloperoxidase) comprises an amino acid sequence of Formula (VII): GTPX₁₈, wherein X₁₈ is proline or valine. In an embodiment, the amino acid sequence of Formula (VII) is selected from GTPP and GTPV. In an embodiment, the peroxidase (e.g. haloperoxidase) comprises an amino acid sequence of Formula (VII-a): X₁₉LIGTPX₁₈, wherein X₁₉ is proline or valine and X₁₈ is asparagine or lysine. In an embodiment, the amino acid sequence of Formula (VII-a) is selected from NLIGTPP, KLIGTPP, NLIGTPV, and KLIGTPV. In an embodiment, the peroxidase (e.g. haloperoxidase) comprises an amino acid sequence of Formula (VII-b): DGSX₁₉LIGTPX₁₈, wherein X₁₈ is proline or valine and X₁₉ is asparagine or lysine. In an embodiment, the amino acid sequence of Formula (VII-b) is selected from DGSNLIGTPP, DGSKLIGTPP, DGSNLIGTPV, and DGSKLIGTPV. In an embodiment, the peroxidase (e.g. haloperoxidase) comprises an amino acid sequence of Formula (VII-c): X₂₀YDGSX₁₉LIGTPX₁₈, wherein X₁₈ is proline or valine, X₁₉ is asparagine or lysine, and X₂₀ is alanine or glycine. In an embodiment, the amino acid sequence of Formula (VII-c) is selected from AYDGSNLIGTPP, GYDGSNLIGTPP, AYDGSKLIGTPP, GYDGSKLIGTPP, AYDGSNLIGTPV, GYDGSNLIGTPV, AYDGSKLIGTPV and GYDGSKLIGTPV.

In an embodiment, the peroxidase (e.g. haloperoxidase) comprises an amino acid sequence of Formula (VIII): AYLNACX_{2I}I, wherein X₂₁ is leucine or isoleucine. In an embodiment, the amino acid sequence of Formula (VIII) is selected from AYLNACLI and AYLNACII. In an embodiment, the peroxidase (e.g. haloperoxidase) comprises an amino acid sequence of Formula (VIII-a): AYLNACX₂₁IX₂₂L, wherein X₂₁ is leucine or isoleucine and X₂₂ is leucine or methionine. In an embodiment, the amino acid sequence of Formula (VIII-a) is selected from AYLNACLILL, AYLNACIILL, AYLNACLIML, and AYLNACIIML.

In an embodiment, the peroxidase (e.g. haloperoxidase) comprises an amino acid sequence of Formula (IX): IPX₂₃ or LPX₂₃, wherein X₂₃ is methionine or phenylalanine. In an embodiment, the amino acid sequence of Formula (IX) is selected from IPM, IPF, LPM, and LPF. In an embodiment, the peroxidase (e.g. haloperoxidase) comprises an amino acid sequence of Formula (IX-a): DX₂₅X₂₄IPX₂₃ or DX₂₅X₂₄LPX₂₃, wherein X₂₃ is methionine or phenylalanine; X₂₄ is glycine, histidine, or asparagine; X₂₅ is glutamine, lysine, proline or serine.

In an embodiment, the amino acid sequence of Formula (IX-a) is selected from DQGIPM, DQHIPF, DQNIPM, DQGIPF, DQHIPM, DQNIPF, DQGIPM, DKHIPF, DKNIPM, DKGIPF, DKHIPM, DKNIPF, DKGIPM, DPHIPF, DPNIPM, DPGIPF, DPHIPM, DPNIPF, DPGIPM, DSHIPF, DSNIPM, DSGIPF, DSHIPM, and DSNIPF. In an embodiment, the amino acid sequence of Formula (IX-a) is selected from DQGLPM, DQHLPF, DQNLPM, DQGLPF, DQHLPM, DQNLPF, DQGLPM, DKHLPF, DKNLPM, DKGLPF, DKHLPM, DKNLPF, DKGLPM, DPHLPF, DPNLPM, DPGLPF, DPHLPM, DPNLPF, DPGLPM, DSHLPF, DSNLPM, DSGLPF, DSHLPM, and DSNLPF.

In an embodiment, the peroxidase (e.g. haloperoxidase) comprises an amino acid sequence of Formula (X): DX₂₆Q, wherein X₂₆ is histidine, lysine or asparagine. In an embodiment, the amino acid sequence of Formula (X) is selected from DHQ, DKQ, and DNQ.

In an embodiment, the peroxidase (e.g. haloperoxidase) comprises an amino acid sequence of Formula (X-a): DX₂₆QX₂₇GF or DX₂₆QX₂₇VF, wherein X₂₆ is histidine, lysine or asparagine; and X₂₇ is arginine, glutamine, or aspartate. In an embodiment, the amino acid sequence of Formula (X-a) is selected from DHQRGF, DHQQGF, DHQDGF, DKQRGF, DKQQGF, DKQDGF, DNQRGF, DNQQGF, and DNQDGF.

In an embodiment, the peroxidase (e.g. haloperoxidase) comprises an amino acid sequence of Formula (XI): EVATRALKX₂₈X₂₉R, wherein X₂₈ is alanine or glycine and X₂₉ is phenylalanine or tyrosine. In an embodiment, the amino acid sequence of Formula (XI) is selected from EVATRALKAFR, EVATRALKAYR, EVATRALKGFR, and EVATRALKGYR.

In an embodiment, the peroxidase (e.g. haloperoxidase) comprises an amino acid sequence of Formula (XI-a): EVATRALKX₂₈X₂₉RX₃₀QK, wherein X₂₈ is alanine or glycine, X₂₉ is phenylalanine or tyrosine, and X₃₀ is phenylalanine or tyrosine. In an embodiment, the amino acid sequence of Formula (XI-a) is selected from EVATRALKAFRYQK, EVATRALKAYRFQK, EVATRALKAFRFQK, EVATRALKAYRYQK, EVATRALKGFRYQK, EVATRALKGYRFQK, EVATRALKGFRFQK, and EVATRALKGYRYQK. In an embodiment, the peroxidase (e.g. haloperoxidase) comprises an amino acid sequence of Formula (XI-b): HRRLRPEAX₃₁G, wherein X₃₁ is valine, isoleucine or threonine. In an embodiment, the amino acid sequence of Formula (XI-b) is selected from HRRLRPEAVG, HRRLRPEAIG, and HRRLRPEATG.

In an embodiment, the peroxidase (e.g. haloperoxidase) comprises an amino acid sequence of Formula (XII): EGSPX₃₂HP, wherein X₃₂ is methionine or phenylalanine. In an embodiment, the amino acid sequence of Formula (XII) is selected from EGSPMHP and EGSPFHP. In an embodiment, the peroxidase (e.g. haloperoxidase) comprises an amino acid sequence of Formula (XII-a): EGSPX₃₂HPX₃₃YG, wherein X₃₂ is methionine or phenylalanine, and X₃₃ is serine or alanine. In an embodiment, the amino acid sequence of Formula (XII-a) is selected from EGSPMHPSYG, EGSPMHPAYG, EGSPFHPSYG, and EGSPFHPAYG. In an embodiment, the peroxidase (e.g. haloperoxidase) comprises an amino acid sequence of Formula (XII-b): EGSPX₃₂HPX₃₃YGX₃₄GHA, wherein X₃₂ is methionine or phenylalanine, X₃₃ is serine or alanine, and X₃₄ is serine or alanine. In an embodiment, the amino acid sequence of Formula (XII-b) is selected from EGSPMHPSYGAGHA, EGSPMHPAYGSGHA, EGSPMHPSYGAGHA, EGSPMHPAYGSGHA, EGSPFHPSYGAGHA, EGSPFHPAYGSGHA, EGSPFHPSYGAGHA, and EGSPFHPAYGSGHA.

In an embodiment, the peroxidase (e.g. haloperoxidase) comprises an amino acid sequence of Formula (XIII): VAGACX₃₅T, wherein X₃₅ is valine or threonine. In an embodiment, the amino acid sequence of Formula (XIII) is selected from VAGACVT and VAGACTT. In an embodiment, the peroxidase (e.g. haloperoxidase) comprises an amino acid sequence of Formula (XIII-a): VAGACX₃₅TLKAFFX₃₆, wherein X₃₅ is valine or threonine and X₃₆ is glutamine or aspartate. In an embodiment, the amino acid sequence of Formula (XIII-a) is selected from VAGACVTLKAFFQ, VAGACVTLKAFFD, VAGACTTLKAFFQ, and VAGACTTLKAFFD.

In an embodiment, the peroxidase (e.g. haloperoxidase) comprises an amino acid sequence of Formula (XIV): DGX₃₇X₃₈LD or DEX₃₇X₃₈LD, wherein X₃₇ is serine, lysine, arginine, threonine or aspartate; and X₃₈ is arginine, lysine or glycine. In an embodiment, the amino acid sequence of Formula (XIV) is selected from DGSRLD, DGSKLD, DGSGLD, DGKRLD, DGKKLD, DGKGLD, DGRRLD, DGRKLD, DGRGLD, DGTRLD, DGTKLD, DGTGLD, DGDRLD, DGDKLD, and DGDGLD. In an embodiment, the amino acid sequence of Formula (XIV) is selected from DESRLD, DESKLD, DESGLD, DEKRLD, DEKKLD, DEKGLD, DERRLD, DERKLD, DERGLD, DETRLD, DETKLD, DETGLD, DEDRLD, DEDKLD, and DEDGLD.

In an embodiment, the peroxidase (e.g. haloperoxidase) comprises an amino acid sequence of Formula (XV): LTVX₃₉, wherein X₃₉ is alanine, aspartate or glutamate. In an embodiment, the amino acid sequence of Formula (XV) is selected from LTVA, LTVD, and LTVE. In an embodiment, the peroxidase (e.g. haloperoxidase) comprises an amino acid sequence of Formula (XV-a): X₄₀ELNK, wherein X₄₀ is glycine or aspartate. In an embodiment, the amino acid sequence of Formula (XV-a) is selected from GELNK and DELNK. In an embodiment, the peroxidase (e.g. haloperoxidase) comprises an amino acid sequence of Formula (XV-b): LTVX₃₉X₄₀ELNK, wherein X₃₉ is alanine, aspartate or glutamate; and X₄₀ is glycine or aspartate. In an embodiment, the amino acid sequence of Formula (XV-b) is selected from LTVAGELNK, LTVDGELNK, LTVEGELNK, LTVADELNK, LTVDDELNK, and LTVEDELNK.

In an embodiment, Formula (XVI) comprises NISIGR, NISVGR, or NVAIGR.

In an embodiment, the peroxidase (e.g. haloperoxidase) comprises an amino acid sequence of Formula (XVII): AGVHYX₄₁X₄₂D, wherein X₄₁ is phenylalanine or tyrosine and X₄₂ is threonine or serine. In an embodiment, the amino acid sequence of Formula (XVII) is selected from AGVHYFSD, AGVHYFTD, AGVHYYSD, and AGVHYYTD. In an embodiment, the peroxidase (e.g. haloperoxidase) comprises an amino acid sequence of Formula (XVII-a): AGVHYX₄₁X₄₂DX₄₃X₄₄ES, wherein X₄₁ is phenylalanine or tyrosine; X₄₂ is threonine or serine; X₄₃ is tyrosine or glutamine; and X₄₄ is isoleucine, arginine, phenylalanine, or valine.

In an embodiment, the amino acid sequence of Formula (XVII-a) is selected from AGVHYFTDYIES, AGVHYFSDYIES, AGVHYFTDQIES, AGVHYFSDQIES, AGVHYFTDYRES, AGVHYFSDYRES, AGVHYFTDQRES, AGVHYFSDQRES, AGVHYYTDYFES, AGVHYYSDYFES, AGVHYYTDQFES, AGVHYYSDQFES, AGVHYYTDYVES, AGVHYYSDYVES, AGVHYYTDQVES, and AGVHYYSDQVES.

In an embodiment, the peroxidase (e.g. haloperoxidase) comprises an amino acid sequence of Formula (XVIII): EQX₄₅LT, wherein X₄₅ is methionine, lysine, or serine. In an embodiment, the amino acid sequence of Formula (XVIII) is selected from EQMLT, EQKLT, and EQSLT. In an embodiment, the peroxidase (e.g. haloperoxidase) comprises an amino acid sequence of Formula (XVIII-a): LX₄₆EQX₄₅LT, wherein X₄₅ is methionine, lysine, or serine; and X₄₆ is glutamate, glutamine or lysine. In an embodiment, the amino acid sequence of Formula (XVIII-a) is selected from LEEQMLT, LQEQMLT, LKEQMLT, LEEQKLT, LQEQKLT, LKEQKLT, LEEQSLT, LQEQSLT, and LKEQSLT.

In an embodiment, the peroxidase (e.g. haloperoxidase) comprises an amino acid sequence of Formula (XIX): GEX₄₇X₄₈A, wherein X₄₇ is glutamine, lysine or glutamate; and X₄s is isoleucine or valine. In an embodiment, the amino acid sequence of Formula (XIX) is selected from GEQIA, GEQVA, GEKIA, GEKVA, GEEIA, and GEEVA. In an embodiment, the peroxidase (e.g. haloperoxidase) comprises an amino acid sequence of Formula (XIX-a): GEX₄₇X₄₈AX₄₉G, wherein X₄₇ is glutamine, lysine or glutamate; X₄₈ is isoleucine or valine; and X₄₉ is leucine or isoleucine. In an embodiment, the amino acid sequence of Formula (XIX-a) is selected from GEQIALG, GEQIAIG, GEQIALG, GEQIAIG, GEKIALG, GEKIAIG, GEKVALG, GEKVAIG, GEEVALG, GEEVAIG, GEEVALG, and GEEVAIG.

In an embodiment, the peroxidase (e.g. haloperoxidase) comprises an amino acid sequence of Formula (XX): LX₅₀EQ, wherein X₅₀ is glutamate, glutamine or lysine. In an embodiment, the amino acid sequence of Formula (XX) is selected from LEEQ, LQEQ, and LKEQ. In an embodiment, the peroxidase (e.g. haloperoxidase) comprises an amino acid sequence of Formula (XX-a): LX₅₀EQX₅₁LT, wherein X₅₀ is glutamate, glutamine or lysine; and X₅₁ is methionine, lysine or serine. In an embodiment, the amino acid sequence of Formula (XX-a) is selected from LEEQMLT, LEEQKLT, LEEQSLT, LQEQMLT, LQEQKLT, LQEQSLT, LKEQMLT, LKEQKLT, and LKEQSLT. In an embodiment, the peroxidase (e.g. haloperoxidase) comprises an amino acid sequence of Formula (XX-b): LX₅₀EQX₅iLTFX₅₂E or LX₅₀EQX₅iLTYX₅₂E, wherein X₅₀ is glutamate, glutamine or lysine; X₅₁ is methionine, lysine or serine; and X₅₂ is proline, alanine, glycine or serine. In an embodiment, the amino acid sequence of Formula (XX-b) is selected from LEEQMLTPE, LEEQMLTAE, LEEQMLTGE, LEEQMLTSE, LEEQKLTPE, LEEQKLTAE, LEEQKLTGE, LEEQKLTSE, LEEQSLTPE, LEEQSLTAE, LEEQSLTGE, LEEQSLTSE, LQEQMLTPE, LQEQMLTAE, LQEQMLTGE, LQEQMLTSE, LQEQKLTPE, LQEQKLTAE, LQEQKLTGE, LQEQKLTSE, LQEQSLTPE, LQEQSLTAE, LQEQSLTGE, LQEQSLTSE, LKEQMLTPE, LKEQMLTAE, LKEQMLTG, LKEQMLTSE, LKEQKLTPE, LKEQKLTAE, LKEQKLTGE, LKEQKLTSE, LKEQSLTPE, LKEQSLTAE, LKEQSLTGE, and LKEQSLTSE.

In an embodiment, the peroxidase (e.g. haloperoxidase) comprises an amino acid sequence of Formula (XXI): EX₅₃F, wherein X₅₃ is threonine, glutamate, aspartate, lysine or asparagine. In an embodiment, the amino acid sequence of Formula (XXI) is selected from ETF, EEF, EDF, EKF, or ENF. In an embodiment, the peroxidase (e.g. haloperoxidase) comprises an amino acid sequence of Formula (XXI-a): EX₅₃FX₅₄M or EX₅₃FX₅₄F, wherein X₅₃ is threonine, glutamate, aspartate, lysine or asparagine; and X₅₄ is threonine, serine or phenylalanine. In an embodiment, the amino acid sequence of Formula (XXI-a) is selected from ETFTM, EEFMT, EDFTM, EKFTM, ENFTM, ETFSM, EEFSM, EDFSM, EKFSM, ENFSM, ETFFM, EEFFM, EDFFM, EKFFM, and ENFFM. In an embodiment, the amino acid sequence of Formula (XXI-a) is selected from ETFTF, EEFMF, EDFTF, EKFTF, ENFTF, ETFSF, EEFSF, EDFSF, EKFSF, ENFSF, ETFFF, EEFFF, EDFFF, EKFFF, and ENFFF.

In an embodiment, the peroxidase (e.g. haloperoxidase) comprises an amino acid sequence of Formula (XXII): PSGHAX₅₅FX₅₆, wherein X₅₅ is serine or threonine, and X₅₆ is glycine or serine. In an embodiment, the amino acid sequence of Formula (XXII) is selected from PSGHASFG, PSGHASFS, PSGHASFG, PSGHASFS, PSGHATFG, PSGHATFS, PSGHATFG, and PSGATFS. In an embodiment, the peroxidase (e.g. haloperoxidase) comprises an amino acid sequence of Formula (XXII-a): FPX₅₇YPSGHAX₅₅FX₅₆, wherein X₅₅ is serine or threonine, X₅₆ is glycine or serine, and X₅₇ is proline or asparagine. In an embodiment, the amino acid sequence of Formula (XXII) is selected from FPNYPSGHASFG, FPNYPSGHASFS, FPNYPSGHASFG, FPNYPSGHASFS, FPNYPSGHATFG, FPNYPSGHATFS, FPNYPSGHATFG, FPNYPSGATFS, FPPYPSGHASFG, FPPYPSGHASFS, FPPYPSGHASFG, FPPYPSGHASFS, FPPYPSGHATFG, FPPYPSGHATFS, FPPYPSGHATFG, and FPPYPSGATFS.

The peroxidase (e.g., haloperoxidase) of the present disclosure may contain any of the amino acid sequences described herein. In an embodiment, the peroxidase (e.g., haloperoxidase) comprises an amino acid sequence of one of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of two of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), or (XX-a), (XX-b), (XXI), (XXI-a), (XXII), or (XXII-a). In an embodiment, the peroxidase (e.g., haloperoxidase) comprises an amino acid sequence of three of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of four of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of five of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), or (XX-a), (XX-b), (XXI), (XXI-a), (XXII), or (XXII-a). In an embodiment, the peroxidase (e.g., haloperoxidase) comprises an amino acid sequence of six of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), or (XX-a), (XX-b), (XXI), (XXI-a), (XXII), or (XXII-a). In an embodiment, the peroxidase (e.g., haloperoxidase) comprises an amino acid sequence of seven of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of eight of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of nine of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 10 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 11 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 12 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 13 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 14 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 15 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 16 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 17 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 18 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 19 of: Formulas (I), (I-a), (II), (II-a), (II-b), (III), (III-a), (IIIb), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 20 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 20 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 21 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a). In an embodiment, the peroxidase (e.g., haloperoxidase) comprises an amino acid sequence of 22 of: Formulas (I), (I-a), (II), (II-a), (II-b), (III), (III-a), (III-b), (IV), (IV-a), (IV-b), (IV-c), (V), (V-a), (V-b), (VI), (VI-a), (VI-b), (VII), (VII-a), (VII-b), (VII-c), (VIII), (VIII-a), (IX), (IX-a), (X), (X-a), (XI), (XI-a), (XI-b), (XII), (XII-a), (XII-b), (XIII), (XIII-a), (XIV), (XV), (XV-a), (XV-b), (XVI), (XVII), (XVII-a), (XVIII), (XVIII-a), (XIX), (XIX-a), (XX), (XX-a), (XX-b), (XXI), (XXI-a), (XXII), or (XXII-a).

In an embodiment, the peroxidase (e.g., haloperoxidase) comprises an amino acid sequence of one of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of two of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of three of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of four of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII). In an embodiment, the peroxidase (e.g., haloperoxidase) comprises an amino acid sequence of five of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of six of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of seven of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of eight of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of nine of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of 10 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII). In an embodiment, the peroxidase (e.g., haloperoxidase) comprises an amino acid sequence of 11 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of 12 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of 13 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of 14 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII). In an embodiment, the peroxidase (e.g., haloperoxidase) comprises an amino acid sequence of 15 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), or (XXII). In an embodiment, the peroxidase (e.g., haloperoxidase) comprises an amino acid sequence of 16 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or or (XXII). In an embodiment, the peroxidase (e.g., haloperoxidase) comprises an amino acid sequence of 17 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or or (XXII). In an embodiment, the peroxidase (e.g., haloperoxidase) comprises an amino acid sequence of 18 of Formula (I), (II), (III), (IV), (V), (VI), (VII), (VIII), (IX), (X), (XI), (XII), (XIII), (XIV), (XV), (XVI), (XVII), (XVIII), (XIX) (XX), (XXI) or (XXII). In an embodiment, the peroxidase (e.g., haloperoxidase) comprises an amino acid sequence of 19 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or or (XXII). In an embodiment, the peroxidase (e.g., haloperoxidase) comprises an amino acid sequence of 20 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII). In an embodiment, the peroxidase (e.g., haloperoxidase) comprises an amino acid sequence of 21 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII). In an embodiment, the peroxidase (e.g., haloperoxidase) comprises an amino acid sequence of 22 of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII).

TABLE 1
Summary table of exemplary motifs and associated
subvariables, Xₙ in haloperoxidases

| Motif (Formula or | | |
|---|---|---|
| Subformula) | Amino Acid Sequence | Subvariable, Xₙ |
| Formula (I) | TGPX₁ | X₁ is P or T |
| Formula (I-a) | GPX₂TGPX₁ | X₁ is P or T |
| | | X₂ is Q or L |
| Formula (II) | X₃WKE | X₃ is S or A |
| Formula (II-a) | GX₄FX₃WKE | X₃ is S or A |
| | | X₄ is I or V |
| Formula (II-b) | GIX₅X₃WX₆ | X₃ is S or A; |
| | | X₅ is F or L; and |
| | | X₆ is K, E or Q |
| Formula (III) | X₇KW or X₇KY | X₇ is E or Q |
| Formula (III-a) | X₇KWX₈FE | X₇ is E or Q; and |
| | | X₈ is E, R, or H |
| Formula (III-b) | X-KWX₈FEFW or | X₇ is E or Q; and |
| | X-KYX₈FEFW | X₈ is E, R, or H |
| Formula (IV) | YHX₉ | X₉ is G or A |
| Formula (IV-a) | YHX₉PFY | X₉ is G or A |
| Formula (IV-b) | X₁₀YHGPFY or | X₁₀ is Q, G, M, or I |
| | X₁₀YHAPFY | |
| Formula (IV-c) | FRX₁₁YHX₉PFY | X₉ is G or A, and |
| | | X₁₁ is Q, G, M, or I |
| Formula (V) | GVHX₁₂ | X₁₂ is W or Y |
| Formula (V-a) | GVHWX₁₃F | X₁₃ is R, H, V or E |
| Formula (V-b) | WX₁₃FDAX₁₄ | X₁₃ is R, H, V or E; |
| | | and X₁₄ is A or F |
| Formula (VI) | X₁₅LI | X₁₅ is N or K |
| Formula (VI-a) | DGX₁₆X₁₅LI | X₁₅ is N or K, and |
| | | X₁₆ is S or A |
| Formula (VI-b) | WX₁₇YDGX₁₆X₁₅LI | X₁₅ is N or K, X₁₆ |
| | | is S or A, and |
| | | X₁₇ is A or G |
| Formula (VII) | GTPX₁₈ | X₁₈ is P or V |
| Formula (VII-a) | X₁₉LIGTPX₁₈ | X₁₈ is P or V, and |
| | | X₁₉ is N or K |
| Formula (VII-b) | DGSX₁₉LIGTPX₁₈ | X₁₈ is P or V, and |
| | | X₁₉ is N or K |
| Formula (VII-c) | X₂₀YDGSX₁₉LIGTPX₁₈ | X₁₈ is P or V, |
| | | X₁₉ is N or K, and |
| | | X₂₀ is A or G |
| Formula (VIII) | AYLNACX₂₁I | X₂₁ is L or I |
| Formula (VIII-a) | AYLNACX₂₁IX₂₂L | X₂₁ is L or I and |
| | | X₂₂ is L or M |
| Formula (IX) | IPX₂₃ or LPX₂₃ | X₂₃ is Mor F |
| Formula (IX-a) | DX₂₅X₂₄IPX₂₃ or | X₂₃ is M or F; |
| | DX₂₅X₂₄LPX₂₃ | X₂₄ is G, H, or N; |
| | | X₂₅ is Q, K, P or S |
| Formula (X) | DX₂₆Q | X₂₆ is H, K or N |
| Formula (X-a) | DX₂₆QX₂₇GF or | X₂₆ is H, K or N; and |
| | DX₂₆QX₂₇VF | X₂₇ is R, Q, or D |
| Formula (XI) | EVATRALKX₂₈X₂₉R | X₂₈ is A or G, |
| | | X₂₉ is F or Y |
| Formula (XI-a) | EVATRALKX₂₈X₂₉RX₃₀QK | X₂₈ is A or G, |
| | | X₂₉ is F or Y, and |
| | | X₃₀ is F or Y |
| Formula (XI-b) | HRRLRPEAX₃₁G | X₃₁ is V, I or T |
| Formula (XII) | EGSPX₃₂HP | X₃₂ is M or F |
| Formula (XII-a) | EGSPX ₃₂HPX₃₃YG | X₃₂ is M or F, and |
| | | X₃₃ is S or A |
| Formula (XII-b) | EGSPX₃₂HPX₃₃YGX₃₄GHA | X₃₂ is M or F, |
| | | X₃₃ is S or A, and |
| | | X₃₄ is S or A |
| Formula (XIII) | VAGACX ₃₅T | X₃₅ is V or T |
| Formula (XIII-a) | VAGACX₃₅TLKAFFX₃₆, | X₃₅ is V or T and |
| | | X₃₆ is Q or D |
| Formula (XIV) | DGX₃₇X₃₈LD or | X₃₇ is S, K, R, |
| | DEX₃₇X₃₈LD | T or D; and |
| | | X₃₈ is R, K or G |
| Formula (XV) | LTVX₃₉ | X₃₉ is A, D or E |
| Formula (XV-a) | X₄₀ELNK | X₄₀ is G or D |
| Formula (XV-b) | LTVX₃₉X₄₀ELNK | X₃₉ is A, D or E; and |
| | | X₄₀ is G or D |
| Formula (XVI) | NISIGR, NISVGR, or | |
| | NVAIGR | |
| Formula (XVII) | AGVHYX₄₁X₄₂D | X₄₁ is F or Y; and |
| | | X₄₂ is T or S; |
| Formula (XVII-a) | AGVHYX₄₁X₄₂DX₄₃X₄₄ES | X₄₁ is F or Y; |
| | | X₄₂ is T or S; |
| | | X₄₃ is Y or Q; and |
| | | X₄₄ is I, R, F, or V |
| Formula (XVIII) | EQX₄₅LT | X₄₅ is M, K, or S |
| Formula | LX₄₆EQX₄₅LT | X₄₅ is M, K, or S; and |
| (XVIII-a) | | X₄₆ is E, Q or K |
| Formula (XIX) | GEX₄₇X₄₈A | X₄₇ is Q, K or |
| | | E; and X₄₈ is |
| | | I or V |
| Formula (XIX-a) | GEX₄₇X₄₈AX₄₉G | X₄₇ is Q, K or E; |
| | | X₄₈ is I or V; and |
| | | X₄₉ is L or I |
| Formula (XX) | LX₅₀EQ | X₅₀ is E, Q or K. |
| Formula (XX-a) | LX₅₀EQX₅₁LT | X₅₀ is E, Q or K; and |
| | | X₅₁ is M, K or S |
| Formula (XX-b) | LX₅₀EQX₅₁LTFX₅₂E or | X₅₀ is E, Q or K; |
| | | X₅₁ is M, K or S; and |
| | LX₅₀EQX₅₁LTYX₅₂E | X₅₂ is P, A, G or S |
| Formula (XXI) | EX₅₃F | X₅₃ is T, E, D, K or N |
| Formula (XXI-a) | EX₅₃FX₅₄M or EX₅₃FX₅₄F | X₅₃ is T, E, D, |
| | | K or N; and |
| | | X₅₄ is T, S or F |
| Formula (XXII) | PSGHAX₅₅FX₅₆ | X₅₅ is S or T, and |
| | | X₅₆ is G or S |
| Formula (XXII-a) | FPX₅₇YPSGHAX₅₅FX₅₆ | X₅₅ is S or T, |
| | | X₅₆ is G or S, and |
| | | X₅₇ is P or N |

The sequences of exemplary haloperoxidases found in various organisms including Curvularia inaequalis, Corallima officinalis, Halomicronema hongdechloris, Moore bouillonii, Trichodesmium erythraeum, Aphanocapsa montana, Synechococcus sp. PCC7335, Coralilinand Saccharomyces cerevisiae are provided below in Table 2. These sequences include both naturally occurring and engineered VHPO sequences.

TABLE 2
Exemplary VHPO sequences

| Sequence number | Sequence |
|---|---|
| SEQ ID NO: 1 | MGSVTPIPLPKIDEPEEYNTNYILFWNHVGLELNRVTHTVGGPLT |
| C. inaequalis | GPPLSARALGMLHLAIHDAYFSICPPTDFTTFLSPDTENAAYRLPS |
| | PNGANDARQAVAGAALKMLSSLYMKPVEQPNPNPGANISDNA |
| | YAQLGLVLDRSVLEAPGGVDRESASFMFGEDVADVFFALLNDP |
| | RGASQEGYHPTPGRYKFDDEPTHPVVLIPVDPNNPNGPKMPFRQ |
| | YHAPFYGKTTKRFATQSEHFLADPPGLRSNADETAEYDDAVRV |
| | AIAMGGAQALNSTKRSPWQTAQGLYWAYDGSNLIGTPPRFYNQ |
| | IVRRIAVTYKKEEDLANSEVNNADFARLFALVDVACTDAGIFSW |
| | KEKWEFEFWRPLSGVRDDGRPDHGDPFWLTLGAPATNTNDIPF |
| | KPPFPAYPSGHATFGGAVFQMVRRYYNGRVGTWKDDEPDNIAI |
| | DMMISEELNGVNRDLRQPYDPTAPIEDQPGIVRTRIVRHFDSAW |
| | ELMFENAISRIFLGVHWRFDAAAARDILIPTTTKDVYAVDNNGA |
| | TVFQNVEDIRYTTRGTREDEEGLFPIGGVPLGIEIADEIFNNGLKP |
| | TPPEIQPMPQETPVQKPVGQQPVKGMWEEEQAPVVKEAP |
| SEQ ID NO: 2 | MGSVTPIPLPKIDEPEEYNTNYILFWNHVGLELNRVTHTVGGPLT |
| C. inaequalis | GPPLSARALGMLHLAIHDAYFSICPPTDFTTFLSPDTENAAYRLPS |
| | PNGANDARQAVAGAALKMLSSLYMKPVEQPNPNPGANISDNA |
| | YAQLGLVLDRSVLEAPGGVDRESASFMFGEDVADVFFALLNDP |
| | RGASQEGYHPTPGRYKFDDEPTHPVVLIPVDPNNPNGPKMPFRQ |
| | YHAPFYGKTTKRFATQSEHFVADPPGLRSNADETAEYDDAVRV |
| | AIAMGGAQALNSTKRSPWQTAQGLYWAYDGSNLIGTPPRFYNQ |
| | IVRRIAVTYKKEEDLANSEVNNADFARLFALVDVACADAGIFSW |
| | KEKWEFEFWRPLSGVRDDGRPDHGDPFWLTLGAPATNTNDIPF |
| | KDPFPAYPSGHATFGGAVFQMVRRYYNGRVGTWKDDEPDNIAI |
| | DMMISEELNGVNRDLRQPYDPTAPIEDQPGIVRTRIVRHFDSAW |
| | ELMFENAISRIFLGVHWRFDAAAARDILIPTTTKDVYAVDNNGA |
| | TVFQNVEDIRYTTRGTREDEEGLFPIGGVPLGIEIADEIFNNGLKP |
| | TPPEIQPMPQETPVQKPVGQQPVKGMWEEEQAPVVKEAP |
| SEQ ID NO: 3 | MGSVTPIPLPKIDEPEEYNTNYILFWNHVGLELNRVTHTVGGPLT |
| C. inaequalis | GPPLSARALGMLHLAIHDAYFSICPPTDFTTFLSPDTENAAYRLPS |
| | PNGANDARQAVAGAALKMLSSLYMKPVEQPNPNPGANISDNA |
| | YAQLGLVLDRSVLEAPGGVDRESASFMFGEDVADVFFALLNDP |
| | RGASQEGYHPTPGRYKFDDEPTHPVVLIPVDPNNPNGPKMPFRQ |
| | YHAPFYGKTTKRFATQSEHFVADPPGLRSNADETAEYDDAVRV |
| | AIAMGGAQALNSTKRSPWQTAQGLYWAYDGSNLIGTPPRFYNQ |
| | IVRRIAVTYKKEEDLANSEVNNADFARLFALVDVACADAGIFSW |
| | KEKWEFEFWRPLSGVRDDGRPDHGDPFWLTLGAPATNTNDIPF |
| | KDPFPAYPSGHATFGGAVFQMVRRYYNGRVGTWKDDEPDNIAI |
| | DMMISEELNGVNRDLRQPYDPTAPIEDQPGIVRTRIVRHFDSAW |
| | ELMFENAISRIFLGVHWRFDAAAARDILIPTTTKDVYAVDNNGA |
| | TVFQNVEDIRYTTRGTREDEEGLFPIGGVPLGIEIADEIFNNGLKP |
| | TPPEIQPMPQETPVQKPVGQQPVKGMWEEEQAPVVKEAPLEVLF |
| | QGPHHHHHHHH |
| SEQ ID NO: 4 | MKSRKQQALDVRVAAAKLAYDRPHPPQQPNGEESSYLFDGTDP |
| Synechococcus | ARGSQPSYLANYTKGLPHSEVDGLICDPDDYQLFVKGINSGDPN |
| PCC 7335 | DFIATPLGPKRATPDTPIQWKSSYTQRHGVEVRAWESAGAGLTF |
| | DLEGPDAQAVAMPPAPIIGSDELAAEVAEVYEMALLRDIPFSEFD |
| | SNLEVAASIRRLNKLEWFKGANLPGLSTAAAKRRRSKINSQNAF |
| | RGTTFGDTTGPYLSQFLTRGTPEIGFRYDNVLDGRVAYGAIAFR |
| | NRVRFATPNQDYMTSWEWWLDVQNGADLRGTEAYSSGSNRD |
| | RNRYRYIATPRDLATYVHYDALYQAYLNACLAMLAWGIPFDPG |
| | IPFQAPDSLDHQQGFAQFGGPHVLSLVTEVATRALKAVRYQKFN |
| | IHRRLRPEAIAGRIHQWRNTQPTNLAPVAELSTQINQTLQAVKA |
| | HNKAQNDQYKSSIGGEEFDRDEVYLLPMAFAEGSPMHPSYGAG |
| | HAAVAGACVTILKAFFDHSYVLPGPYVTPSTNGQKLQRVPSEEL |
| | SLTIEGELNKLASNISIGRNWAGVHYFTDYYESVRMGEQIAIGIL |
| | EEQKLTYRENFSMTVPLFDGGTVQI |
| SEQ ID NO: 5 | MGSSHHHHHHLEVLFQGPMKSRKQQALDVRVAAAKLAYDRPH |
| Synechococcus | PPQQPNGEESSYLFDGTDPARGSQPSYLANYTKGLPHSEVDGLIC |
| PCC 7335 | DPDDYQLFVKGINSGDPNDFIATPLGPKRATPDTPIQWKSSYTQR |
| | HGVEVRAWESAGAGLTFDLEGPDAQAVAMPPAPIIGSDELAAE |
| | VAEVYEMALLRDIPFSEFDSNLEVAASIRRLNKLEWFKGANLPG |
| | LSTAAAKRRRSKINSQNAFRGTTFGDTTGPYLSQFLTRGTPEIGF |
| | RYDNVLDGRVAYGAIAFRNRVRFATPNQDYMTSWEWWLDVQ |
| | NGADLRGTEAYSSGSNRDRNRYRYIATPRDLATYVHYDALYQA |
| | YLNACLAMLAWGIPFDPGIPFQAPDSLDHQQGFAQFGGPHVLSL |
| | VTEVATRALKAVRYQKFNIHRRLRPEAIAGRIHQWRNTQPTNLA |
| | PVAELSTQINQTLQAVKAHNKAQNDQYKSSIGGEEFDRDEVYLL |
| | PMAFAEGSPMHPSYGAGHAAVAGACVTILKAFFDHSYVLPGPY |
| | VTPSTNGQKLQRVPSEELSLTIEGELNKLASNISIGRNWAGVHYF |
| | TDYYESVRMGEQIAIGILEEQKLTYRENFSMTVPLFDGGTVQI |
| SEQ ID NO: 6 | MSFYRIAGRVVSQATGAGLSGLRVEVWDKDLIFDDFLAQATTD |
| Halomicronema | AQGAFQVQFDDTSFRDFIWFDRLPDLYFKVFDGNQLIKSTADSV |
| hongdechloris | LWNSRDRNQTVVLPVERAPSLALSPPGARGDGAPPAICLFNPPNI |
| | PGARLWDRRLQALEVRRQAAELARRPAHPEHLSNGEELLYRDT |
| | DNQPSHIANYTKGLPHDDRGLLSDADAYQRFVRGIDSGDTRDFQ |
| | DTPLGLRTGMASNFPIGSLTWPNARNYRCGPGKVPGAGNTFDLE |
| | GPDAQAVTMPPAPTLDSPELLAEMVEVYTMALLRDIPFAKFDSA |
| | PEIQQAIDRLNATDWIQSPDALALSPEEESRLRSPFTRQTVFRGIA |
| | PGDEVGPYLSQFLLVGNTGLGDAQAISDGYIQYGSIRADQRVRV |
| | AAPYRDYMTTWEAWLDVQNGADFRGLELYADDPKFRFMATPR |
| | DLATYVHYDALYEAYLNACLIMLSLGVPFDPGIPFQRADHIDKQ |
| | QGFAHFGGPHILSLVTEVATRALKAVRFQKFNVHRRARPEAIGG |
| | LIHRYASGDRQEFMPVEPLVNGFGESFLEEIADHNRRQNQHSDR |
| | SGDASAGSNSYLLPMAFPEGSPMHPAYGAGHGTVAGACVTILK |
| | AFFDHGYELPVCYQPSVDGAQLESVTLDRPLTVGAELNKLAANI |
| | AIGRNWAGVHYFTDYIESIRLGEAIALGILEEQKLTYGENFSMTIP |
| | LFDGGTIRI |
| SEQ ID NO: 7 | MANRSEKSFDVRLDAAKLARSRDYPTHKANGDEQRHADDQYF |
| Moorea bouillonii | MSFTKGLPHNPDTGLLQDPQDFVEFRRAIDDGFIDPFTDPVRHG |
| | AKFEVVFTGQEYTIKRETDPDLLEEFRQWEAPTAGVVFELNGPD |
| | PQAVTMPPAPPLIDANGKANQELIFEIAEVYELAILRDQPLNDFE |
| | KRAANSKIESSINRLNALDYIRNQTGRPRKVNGRGRLDEQTVFR |
| | GSSPGVEVGPYLSQFLLIGNVDLNGGGNVAEGKITYGALQIDQK |
| | LPIATPNLDYMTNMEDYVLVQRGIKQDRERYVLEKDQNPKLPD |
| | RPARRFISTPRDLATYVHYDALYEAYLNACIILLGMQTPFDPGFD |
| | HLSGGGVAAGNPATRRHAGGFALYGGPHILTLVTEVATRALKA |
| | VRFQKFNNHIRLRPEALAARIELLRRFQELPNEAKASVPRALIKYI |
| | KRFQRELESNGTLNSILELANQNGSGSPNYLLPMAFPEGSPMHP |
| | AYGAGHATVAGACVTMLKAFFDTSAVLVETPVKNPKPGQPRTQ |
| | IRFKRYSPKDQGIVFRAPDPSGYTNGEATLVSERSRYFLTLEGEL |
| | NKLAANISIGRNMAGVHYFTDYYDSLRMGEEIAIGILEEQALTYS |
| | TDPFVLSLPTFDGDVIRIGAR |
| SEQ ID NO: 8 | MRERSQKSYSIRVDAAELARSRQHPIHEANGDEQRYGGDKYFM |
| Trichodesmium | SFTKGLIHNPNTGLLEDPRDFVEFRRAIDNGFIDPFTDRVRHGAK |
| erythraeum | YKVNDQGNIVEEDNPQDLDFRQWEAPTAGVVHELEGPDAQAV |
| | TIPPAPPLLDSTRNTNPELIFEMAEVYELAILRDVPLNNFDNKNTT |
| | DELTASIARLNNLDYVNNQNGRPRKVNQAGELDLQTVFRGSSP |
| | GVEVGPYLSQFLLIGNKGVNGVDNTTTGRIAYGVQQIDQRVPVA |
| | SVQNYMTQWENYVRVQRGIKQERETYVEGNGSHRFIYTPRDLA |
| | TYVHYDALYQAYLNACFILLGMGTPFDPSFDHLSGGGEAAKDP |
| | KTRPHAGGFALYAGPHILTLVTEVATRALKAIRFQKENNHIRLRP |
| | EALAARIDLVRRKNNGEIQESLIPQALSQYVHGFIDKLGSNGYGS |
| | LTTSTLGRIAKIAESSYLLPMAFPEGSPMHPAYGAGHATVAGAC |
| | VTILKAFFDTSTVLIKDSNNEYKFKHQEAQDQFIAFRTNSSGTDLI |
| | TEDINNISEALTLEGELNKLAANISIGRNMAGVHYFSDYYDSIRM |
| | GEQIAIGILQEQALTYSTDPFVMSVPTFDGNIVRIGY |
| SEQ ID NO: 9 | MESRRTQARNIRIEAAHVAFNRAPVVHESNGEELQYRFGPTENN |
| Aphanocapsa | APPEDVVRSGQPSHLTSYTKGLPHREDDGLIAHPTDFQTFVRGID |
| montana | SGDVRDFRDTPLGPLQRSGGQVIGRGAQPGQAIDWSSAFTKSCA |
| | VRDRKGGAVPVRAWESQGAGNTFDLEGPDAQAVTMPPAPVAD |
| | SPELIAEMAEVYEMALLRDVPFAKFEHDGDVQAAIARLNQLEW |
| | FNGSEIVGLSAAAQARRRGAVTIANAFRGTTFGDCVGPYLSQFL |
| | CAGTPELGSGGQTAESATAGFVRYGAMVFQNKVRFATPYRDY |
| | MTTWEWWLDVQNGADLRGTETYEGDCASDRYRFIANPRDLAT |
| | YVHYDALYQAYLNACLSMLALGIPFDPGIPFQGADVMDRQQGF |
| | AHFGGPHILSLVTEVATRALKAVRFQKFNVHRRLRPEAVGGRLH |
| | RMKNSPGNPTFAPFEPMCDRLTEIGLLEAVRGHNARQNTIHDGR |
| | QCDQERDTSYLLPMAFPEGSPMHPAYGAGHATVAGACVTILKA |
| | FFDHRYVLPGPYVYPAPCGTKLEIAAGTPALTVEGELNKLASNIS |
| | IGRNWAGVHYCTDYSESVRLGEAIAIGILEEQKLTYGENFSMSLP |
| | RFDGTTIRI |
| SEQ ID NO: 10 | MESRRTQARNIRIEAAHVAFNRAPVVHESNGEELQYRFGPTENN |
| Lyngbya | APPEDVVRSGQPSHLTSYTKGLPHREDDGLIAHPTDFQTFVRGID |
| confervoides | SGDVRDFRDTPLGPLQRSGGQVIGRGAQPGQAIDWSSAFTKSCA |
| | VRDRKGGAVPVRAWESQGAGNTFDLEGPDAQAVTMPPAPVAD |
| | SPELIAEMAEVYEMALLRDVPFAKFEHDGDVQAAIARLNQLEW |
| | FNGSEIVGLSAAAQARRRGAVTIANAFRGTTFGDCVGPYLSQFL |
| | CAGTPELGSGGQTAESATAGFVRYGAMVFQNKVRFATPYRDY |
| | MTTWEWWLDVQNGADLRGTETYEGDCASDRYRFIANPRDLAT |
| | YVHYDALYQAYLNACLSMLALGIPFDPGIPFQGADVMDRQQGF |
| | AHFGGPHILSLVTEVATRALKAVRFQKFNVHRRLRPEAVGGRLH |
| | RMKNSPGNPTFAPFEPMCDRLTEIGLLEAVRGHNARQNTIHDGR |
| | QCDQERDTSYLLPMAFPEGSPMHPAYGAGHATVAGACVTILKA |
| | FFDHRYVLPGPYVYPAPCGTKLEIAAGTPALTVEGELNKLASNIS |
| | IGRNWAGVHYCTDYSESVRLGEAIAIGILEEQKLTYGENFSMSLP |
| | RFDGTTIRI |
| SEQ ID NO: 11 | MILTLKLTLFFTFNPKMSTSESQTNSVELLLDPNTQLVNINDPSPT |
| Lyngbya sp. PCC | ISVMWDQAAQQAVTETKVGPTVGSRAYSMVHTAMFDAWAAY |
| 8106 | DQVAVATQLGDQLQRPEAENTDAHKIEAMSYSAYRVLTDLFPD |
| | QVEIFDGLMADLGFDPNNTTTDTTTAAGIGNVSANALLEFRHQD |
| | GSNQLNDYANTTDYQPVNTPEQLVDIESWTPEHIPVDNPDAPLQ |
| | EYLTPQWGDVTPFGLDSGDQLRPEAPEPFLLVEGEVDLEARTITL |
| | ADGAVVDINRDIVGTIINPEFIAQAEQVVETSANLTDEQKLIAEF |
| | WEDGGGTSFPPGTWMTFGQYTSAQNDHSLDQDAKLFFGLGNA |
| | VFDAGVATWEAKTHYEYTRPVRLVRELGDLGLIGEFNEELGGY |
| | AVEAWAGPGEGTQTILASDFTTYQTPGSHVSPPFAEYVSGHSTFS |
| | ASAATILELVTGSDNFGASVTFEPGQSRFEPNLTPQQEVTLAWDT |
| | FSEAADEAGLSRLFGGIHFEDGDLNGRTLGQDVATEVFEQAQFY |
| | IEGGQEQNILFGSRDADELVGTEISEKLYGRAGNDTLAGGLGDD |
| | QLFGGDGDDILRGDRNHRSPGGSVGGDDILSGGFGNDRLGGKG |
| | GNDTLYGDEGDDQLWGDHGDDLLWGGLGNDTVTGGQGNDTF |
| | VVGVGQGTDLIADFNIGKDLIGLVNGLEVSQLSFNVNGNNTEVV |
| | FGDETLAIVDGVTASLTPTDFVAVV |
| SEQ ID NO: 12 | MGIPADNLQSRAKASFDTRVSAAELALARGVVPSLANGEELLYR |
| Corallina | NPDPENGDPSFIVSFTKGLPHDDNGAIIDPDDFLAFVRAINSGDEK |
| officinalis | EIADLTLGPARDPDTGLPIWRSDLANSLELEVRGWENSSAGLTF |
| | DLEGPDAQSIAMPPAPVLTSPELIAEIAELYLMALGREIEFSEFDS |
| | PKNAEYIQFAIDQLNGLEWENTPAMLGDPPAEIRRRRGEVTVGN |
| | LFRGILPGSEVGPYLSQYIIVGSKQIGSATGGNKTLVSPNAADEFD |
| | GEIAYGSITISQRVRIATPGRDFMTDLKVFLDVQDAADFRGFESY |
| | EPGARLIRTIRDLATWVHFDALYEAYLNACLILLANRVPFDPNIP |
| | FQQEDKLDNQDVFVNFGDAHVLSLVTEVATRALKAVRYQKFNI |
| | HRRLRPEATGGLISVNKIAAEKGESVFPEVDLAVEELEDILEKAEI |
| | SNRKQNIADGDPDPDPSFLLPQAFAEGSPFHPSYGSGHAVVAGA |
| | CVTILKAFFDSNFQIDQVFEVDKDEDKLVKSSFKGTLTVAGELN |
| | KLADNIAIGRNMAGVHYFSDQFESILLGEQVAIGILEEQSLTYGE |
| | NFFFNLPKFDGTTIQI |
| SEQ ID NO: 13 | MMKSRKQQSEEIRRNAAELAAARPHPRHHNNGDEQRLHREDG |
| Nostoc carneum | KPSYVGNFTKCLPHDDDGFLIEPRDYEQWLHSIDSGDPRDFRTIR |
| | LGPGPFQPNGDFQYQNPTDPDYSPSCPIKLNWKASYPGGRPCVR |
| | AWESQGAGLTFDLEGPDAQSNTMPPCPSLDSQELIAEMGELYW |
| | MALCRDVPFANWTTDPLIDAARNSLKRLWWFRRDKTLDLNGH |
| | TDVLPESLARRRILSENGSDPVPFSKLFRGVELGTEVGPYVSQFL |
| | LIGNTGINGNDNANTLADGAIAYGAIRIDQQVRVATPNQDYMQT |
| | WEKWLDVQNGADVRGQETYSSGYRFIATPRDIVTYVHYDALY |
| | QTYLNACLWLLGAGAPFDPGLPFRSPDYIDKQQGFAQFGGPHIL |
| | SLVTEVATRALKAVRYQKFNVHRRLRPEALGGWMYQRRKGSA |
| | TVKARLAVLENMEAAFAGSSGIGARLDTKNGMAGNWLLPMAF |
| | SEGSPTHPSYGAGHATVAAACVTILKAWFDSGWELKDKNGKPI |
| | VYVPKADGSALTDVSSYFAPLTLEGELNKLAANISIGRDWAGVH |
| | YYSDYYESFRLGEQVAIGILEEQKLTYGENFSMTVPLLDGTAVRI |
| SEQ ID NO: 14 | MDSILYWNAVALEANRVSHSNGAREQIGPPLSARALGIVHLGM |
| Nocardia iowensis | YDGYIGVLGDSAQYGPYLPDLPPAPPGANPDAAVAGAAEVTLK |
| | SLFPSQKEAFDEALAGAGLSGPGVAEGLAFGRLVAERILADRAG |
| | DPGVGDAGYQPSPERYRHRPDPFNPDQGFHGPFYGARSKLFSA |
| | MERHENRPPPLGNEAYLEVQRVVRVTGIAPELLGSVPQFVIDSLP |
| | PDRRRRTEERTLIGLFWAYDGANGIGTPPRLYNQIVRLVAEDRN |
| | NSTQENARLFALINVAMADAGILAWEQKYAHDLWRPVVGIREH |
| | DLSMGPAADDAANDISTDCQIDWLPLGAPASNSTDRNFTPNFPA |
| | YPSGHATFGAAALHIARRFYGIAKDDMNPDSLAAGITFVSDELN |
| | GITRDNSGTVRPRHVRSFPEGLWQMIVENGRSRVWLGVHWEFD |
| | FAPRPIEIGGLPLGLAIANDIFDTGMMKSPIGPASR |
| SEQ ID NO: 15 | MTDPILLWNEVALEANRVSHTNGKGEQTGPTLSSRALAIVHLA |
| Nostoc minutum | MYDAYAGVTNDPNLPPYLDLSALPSPGTGASVEAAVAAAAHTT |
| | LASLFPSQKPFFDAILAGAGDISNAGHVFGEKVAQALLNDRMND |
| | PGAAAGDYKPSPQRGRHRPDPDNPGQGFHAPFYGARSKGFAITQ |
| | RHMLAKPPLDNSEYLRALREVRVKGIAPELMGTLPENIDDRKRS |
| | IDETLIGIFWAYDGSAELGTPPRLYNQIVRRVAIAKGNTPAQNAS |
| | LFALVNAAMGDAGILAWEQKYLYDFWRPVVGIREHDRSLGPAP |
| | TEADNNISDDCDPLWLPLGAPNTNSRNKNFTPNFPAYPSGHATF |
| | GAAALHITRLFYGISIGDRKPDNLFKDLDFVSEEFNGINTDNRGT |
| | VRPRHNRNFPGGLWQMIIENGRSRVDLGVHWVFDAFAVKNDG |
| | TPDLTKTDTDGKPFGGVPLGIKIAEDIFQAGAGKGPKKSTVLPSS |
| SEQ 1D NO: 16 | MDSILYWNDVALEANRVSHTNGKGEQTGPTLSSRALAMVHLA |
| Deltaproteobacteria | MYDAYAGITNDAVNFPPYLTTLNPPAPGASIAAAVAAAAHATLS |
| bacterium | KLFPSQKAFFDLKHTQAGLQEPGLHDGHAFGLHVAQTLWDDRK |
| | DDPDASDAGHAPSMAHGDHRPDPDNPDQGYHAPFYGANAKCF |
| | AVTARHGLLPPPRQGNEYTKALREVRGKGIAPELMGTLPSGIKA |
| | RTVDETVRGVYWGYDGAAELGTPPRLYNQIVRTVADAQNNNE |
| | GQNARLFALVNAAMADAGILAWEQKYIHNLWRPVVGIREHDD |
| | SMGPAGTGNNDISNNCDPGWLPFGAPSTNSTKKNFTPPFPAYPS |
| | GHATFGAAALQMTRLFYNINNRGPDDDLFEDLTFVSDEMNGISK |
| | DNKGTVRPRHVRNFPNGLWQMIEENGRSRVDLGVHWVFDAFA |
| | VDNSGNMDLTRNIGGVPLGLNIAENIFTTGGGKGPKKSIV |
| SEQ ID NO: 17 | MLPLYTSEEPSELNKHPIFYWNHVALEMNRVTHSLNGPQTGPT |
| Azospirillum sp. | MSSRALGLLHLAIHDAYFAIDKGYKPYLPGLEPVLDAGETAEDA |
| | EQAMAGAAITVLDDLYSRGGAGISVLANHTLLAHLKNQIDNYP |
| | KHIDTLSVSYRFGARVARKILEILGVRPGEPGADSGRYEPKSGRF |
| | FFRDEPLNPVRLLPVDPNNPSGPEKPVRMYHGPFYGTTTRPFAT |
| | HQQHKLADPPKDDDEYQEAVREVVALGGAPGRPKTVRSADQT |
| | VAALFWAYDGANLIGTPPRLYNQIVRQIAWSQVKADAAPGKTT |
| | AEFVRLLALVNVAMADAGRIAWREKYHFEYWRPLTGVREHDA |
| | ASGPGAQQQNGRKKELDKDADPFWEALGAPETNTNKISFKPPFP |
| | AYPSGHATFGAAAFQITRLFYKKERDLQFDANGPDEIAFAFVSD |
| | ELNGISRDLHQPYSQDTPIQDQPGDVRTRVVRHFESLWDAILEN |
| | GASRVFLGVHWRFDAFASEDVIETTNPDGSTVYRDSKAVNYKT |
| | TGTRSGEEGQFPIGGVPLGIAIANDIFEKDMTPPAELGLPTDPAQ |
| | MVVDPASTSRDALTKSSNTNIR |
| SEQ ID NO: 18 | MPMLPKVEEEAGLNAYPVLYWNHVALEMNRITHSLGGPQTGPT |
| Rhodoplanes | MSSRALGLLHLAMHDAYFIALGHNQTSNPPTYLATTDVTSVLD |
| roseus | RTSKNYTGHRDGPSNPVVPGNLANANLALTGAAVTILDLLYGR |
| | QSPNISVVASDTLRSALRRMIGDHGPYIDTLHPAHGLGVTVARA |
| | VFDLLGVKPDEPGADQGRYEPRQGRYCFRDEPVTPVRPAPIDPN |
| | DPSRGTTAARIYHGPFYGSTVKRFAVHDDAGHRLEKWPEPAAP |
| | GAPGEYQNALQEVVHLGGAPGLPKTHRDPDQTVAAYYWAYDG |
| | ANLIGTPPRLYNQIIRTIAWSKKLPDGDELARTSDFVRLFALANT |
| | AMADAGKYAWLEKYRFELWRPLSGIREHDPSTGPDAVAGTGPE |
| | PIAEPHLSAEGADPFWYALGAPETNTDKVSFKPPFPAYPSGHATF |
| | GAAAFQMVRLYYRQRAGSLPSDPADWQAPDDITFTFVSEELNGI |
| | SRDLRQPYDPSKPIEDQQGVVRTRVRRRFVSLWNAIFENALSRIY |
| | LGVHWRFDAADYGDMTNGSGGYKSPSDMRFSHVWTGPRGPQ |
| | AKYPTGGIPLGLGIANDIFHNGMKSPHGEMTADRIATSSSAAGSA |
| | KMTNTNIR |
| SEQ ID NO: 19 | MLILPKTEEDGALNSLPILYWNHVGLEMNRITHSLGGPQAGPTM |
| Arenimonas sp. | SSRALGLLHLAMHDAYFGCLRSPDYGSYLKTLPPLESVPQTHEA |
| | ANLALTGAAVTVLDQLYRFTGSGISRLATDTLTSMLSTCIRDFSD |
| | HIDTLDVAYNFGEQVARAIIGQLGVKPGQLGADQGRYEPRNGR |
| | YYFRDEPANPVRRVDIDPDHPERGQRSTRMYHGPFYGLEVEPFD |
| | TTADDHRLAPPPRELGHGGDQAQDDAYKRALREVIAFGGAPGR |
| | AKTARNADQTVQALYWAYDGANLIGTPPRLYNQIIRKIAWDRK |
| | PNNAEEATAEFVRLFALVNIAMADAGKYAWKEKYQYQLWRPL |
| | SGVREHDSGADPLSITVNAKLDPDADPFWQALGAPETNTNKISF |
| | KPPFPAYPSGHATFGAACFQMARLFYKKRDGLEFDDNAADNIEF |
| | TFVSDELNGINRDLYQPHDPSLPIEEQPGLVRTRVNRRYPSLWHA |
| | IFENALSRIYLGVHWHFDAFALADVEGEPKDPKDTKEGEVCYK |
| | DPSHIQYSNVWTPKEPKRQTSPVGGVPLGLGVANEIWSRWTKR |
| | DEEANKGELPETAPAAEFLPQTEQLLKSSNTNIR |
| SEQ ID NO: 20 | MEGSKNFPGATVVLPRVNEPAEYNQNYILYWNSVALDLNRLVT |
| Candelina | TLSGPLNGPPGASRVLAILHLAIHDAYFAIETSSGTPTYLSSLQNP |
| submexicana | TDPSTKLPPVLGATDARQAVAGAANTVLIQQYTTPSPNVATATT |
| | IQLTQFLQNAVSAFSDLDTLSSGYRFGVAVGNAMLNLLAIKPGE |
| | PGFDQDSYRPRPGKFRFDDDPTNPVRIVPVDINNPTGPTKAFRIY |
| | QAPFYGMTAKRIAVQGQVNGAETEHILADPPTNDLAEYYSAFD |
| | DIYREGGAQTLNTTRRTPEQTVNGLFWAYDGSNLIGTPVRLFNQ |
| | IIRKVAFDKKPAGTADEKTNADFARLFALFNASLADAGIFAWQE |
| | KYCYEFWRPLSGVRKDDTSLADPFWLSLGAPETNSNQVAIKPPF |
| | PSYPSGHASFSGAGFQSARLYYKQRDNLTFAPDEPDNIAFDFISE |
| | ELNGVSRDLRNPYNPNVPITDQLGTVRTRVVRRFHSLWDAMYD |
| | AALSRVFLGVHWRFDAFASKDILASTTVKSDGTTDYKAAADVR |
| | YTTLGPRADRPGQLFPIGGVPLGIGIANDIFQGGLKPTPANIQPSG |
| | RNKCGDGASSAPPKTLNQPSNEPAVKQMAVQL |
| SEQ ID NO: 21 | MMTKKTTPQPSGRWLSSANLSGNSMSIMYKVLTYAHFNFCTTP |
| Parastagonospora | SKISPSIHRIELFIMTTQPPFVPLPLPPVEESKEYGKNYILHWNLCA |
| nodorum | LDLNRVSHSVAGPQTGPPLSARSLGMLHLAIHDAYFAIQSSTAFS |
| | TFLAANAEDAELRLPDLNGATDARQAVAGAAFTLLGLLYLQGG |
| | PTISNNATAQLQAVLDRFLASAPGGVDQASGSYQFGAAVGEAIF |
| | QLLFHPPGVSADGYHPTPGRYKFDDEPTHPVVLVPVDPNDPNGP |
| | KKAIRQYHGPFYGKTAKRVATQSEHMIADPPGLRSNVDEGAEY |
| | DDALRDIIRMGGAIPLNSTKRSPDDTARGHEWAYDGSNLVGTPP |
| | RFYNQIIRNIAVQHKQDADIESEVNNADFARVFALANAAMADA |
| | GVFSWREKWEFEFWRPLSGVRDDGRPDHGDPFFLSLGAPSTNT |
| | NDIPFKPPFPAYPSGHATFGGAMFQMLRRYYNGRVGTWAPNEP |
| | DNICFEMVSDELNGINRDLRQPYDPTTPITEQPGIVRTRMPYKFSS |
| | LWEAIFDNAISRVFLGVHWRFDAAAAKDILIPTDTKDVYATDNT |
| | GATVYQNPEDIRYETMSTREGHEGLLPIGGVPLGMGIANDIWES |
| | GLKPTPKSIQPAPPAEQSKEGEQVVMGLPNAAP |
| SEQ ID NO: 22 | MASFIPVKLPQQVEDEADNQNYILYWNHVGLDLNRVTHTFDGP |
| Alternaria | QTGPPISARALGMLQLAVHDTYFVIKPSSDFKTFLSLDAEDEKYR |
| conjuncta | LPDPAGADNAQQAVAGAAFTMLSMLYMRPAVTPVPASIANSTY |
| | DQLEYIIQQSITNAPGGVDPASPSFTFGKAVAMKFFDLLFHPEGA |
| | SQQGYHPTVGPYKFDDEPTHPVDLVPQDANDPNGPKVPRRQYH |
| | GPFYGTRAKRFATQSEHILADPPAIRSMSDEQAEYDDALREVYT |
| | MGGAPALNSTKRTPHQTVQGMTWAYDGSKLIGTPPRLYNQIVR |
| | KIAVRYKKDSQSLTSEANNADFARLLALVNVALADAGVFSWKE |
| | KWEFEFWRPLSGVRDDILRDPTRVDRGDPFWLTLGAPATNSDA |
| | LPFKPPFPAYPSGHATFGGAAFQMVRRYYNGRIGSWQDDAPDNI |
| | AIDFVSDELNGISRDLRQPYMLEREITDQPGTVRTRVPRQFSSCW |
| | EMMFENAISRIFLGVHWRFDAAAAKDILVPTTKKDIYAVDDAGT |
| | ALFKNPEDIRYKTRGTRQGIEGDYPIGGVPLGMEIANEIFANGLK |
| | PTPPNL |
| SEQ ID NO: 23 | MATFTPIPLPHIEEPAEYNSNYVLYWHHVGLELNRLTHTVGGPQ |
| Pyrenophora | TGPPISARALGMVQLAVHDAYFTIKPSSSFPTFLTSGGDNPAYRL |
| seminiperda | PALNNASDARQAVAGASVNMLSRLYMKPTSNPGTGNPSNPAPG |
| | ISDNAYAQLKYFLDDSVTKAPGGVSKDSSSFIFGQAVANVFFDL |
| | LFHAPGASQDGYHPTPGPYKFDDEPTHPVVLVPNDPNNPTGPKH |
| | PFRQYHAPFYGKTAKRFATQTEHMIADPPGIRSGANEGAEYDDA |
| | IRQTIAMGGATDLNSTKRSPYQTVQGMFWAYDGSNLVGTPPRF |
| | YNQIVRRIAVTYKKEEDLVNSEVNNADFVRLLALVNVACADAG |
| | IFSWKEKWEFEFWRPLSGVRDDGRPDHGDPFWLTLGAPSTNTN |
| | DIPFKPPFPAYPSGHATFGGAVFQMVRRYYNGRIGNWKDNEPD |
| | NIAIDMVVSEELNGLSRDLRQPYDPTAPITDQPGIVRTRIPRHFSS |
| | AWEMMFENAISRIFLGVHWRFDAAAAKDILIPTITKDVYAVDEH |
| | GASLFQNVEDIKYTTMGTRQGHDGLLPIGGVPLGLGIANEIFDN |
| | GLKPTPPEKQPMTQQSPIHQGPPRMKNKETTVVEEQVPIMDIAP |
| SEQ ID NO: 24 | MATFTPIPLPQIDEPAEYNTNYVLYWHHVGLELNRVTHTVGGPQ |
| Alternaria | TGPPISARALGMLQLAVHDAYFAIHPSSSFPTFLTSGADNPAYAL |
| alternata | PELNGADDARQAVAGASVTMLSMLYMKPPTNPNPNPGATISDN |
| | AYAQLQYVIDKSVTDAPGGVDAESSSFNFGKAVATVFFNLLFHA |
| | PGASQAGYHPTPGPYKFDDEPTHPVVLVPVDANNPDGPKRPFRQ |
| | YHGPFYGKTAKRFATQTEHMIADPPAIRSAVGEQAEYDDSVRQI |
| | IAMGGATGLNSTKRSPFQTTQGMFWAYDGSNLVGTPPRFYNQI |
| | VRRIAVTYKKEEDLTNSEVNNADFVRLLALVNVACADAGIFSW |
| | KEKWEFEFWRPLSGVRDDNFRDPNRPDRGDPFWLTLGAPATNT |
| | NDIPFKPPFPAYPSGHATFGGAVFQMVRRYYNGRVGNWKDDEV |
| | DNIAIDMMVSEELNGLSRDLRQPYDPKAPITDQPGIVRTRVPRHF |
| | SSVWEMMFENAISRIFLGVHWRFDAAAAKDILIPTTTKDVYAVD |
| | NNGASLFQNVEDIRYTTMGTREGHDGLLPIGGVPLGIGIANEIFD |
| | TGLKPTPPEKQPVPPPPFNQSGPTKEMLEEAGSEEQVPMMDVAP |
| SEQ ID NO: 25 | MGSVTPIPLPKIDEPEEYNTNYILFWNHVGLELNRVTHTVGGPLT |
| Curvularia | GPPLSARALGMLHLAIHDAYFSICPPTDFTTFLSPDTENAAYRLPS |
| inaequalis | PNGANDARQAVAGAALKMLSSLYMKPVEQPNPNPGANISDNA |
| | YAQLGLVLDRSVLEAPGGVDRESASFMFGEDVADVFFALLNDP |
| | RGASQEGYHPTPGRYKFDDEPTHPVVLIPVDPNNPNGPKMPFRQ |
| | YHAPFYGKTTKRFATQSEHFVADPPGLRSNADETAEYDDAVRV |
| | AIAMGGAQALNSTKRSPWQTAQGLYWAYDGSNLIGTPPRFYNQ |
| | IVRRIAVTYKKEEDLANSEVNNADFARLFALVDVACADAGIFSW |
| | KEKWEFEFWRPLSGVRDDGRPDHGDPFWLTLGAPATNTNDIPF |
| | KDPFPAYPSGHATFGGAVFQMVRRYYNGRVGTWKDDEPDNIAI |
| | DMMISEELNGVNRDLRQPYDPTAPIEDQPGIVRTRIVRHFDSAW |
| | ELMFENAISRIFLGVHWRFDAAAARDILIPTTTKDVYAVDNNGA |
| | TVFQNVEDIRYTTRGTREDEEGLFPIGGVPLGIEIADEIFNNGLKP |
| | TPPEIQPMPQETPVQKPVGQQPVKGMWEEEQAPVVKEAP |
| SEQ ID NO: 26 | MDPVTPIPLPKIEEPEDYNTNYILYWHHVGLEHNRVTHTVGGPQ |
| Stemphylium | TGPPISARALGMLQLAAHDAYFAINPSCDFTTFLTPGAENAAYR |
| lycopersici | LPDLNGANDARQAVAGASIKMLSELYSKPVTQPNPNPGANISDN |
| | AYEQLELVLDRSIADAPGGVDQASASFVFGQAVAEVFFNLLFHP |
| | PGASQEGYHPTPGRYKFDDEPTHPVVLIPVNPNNPDGPKMPFRQ |
| | YHGPFYGTSAKRLGTQTEHMIADPPGIRSAADEGAEYDDSIRVAI |
| | AMGGAIGLNSTKRSPYQTTQGIYWAYDGSNLIGTPPRFYNQIVR |
| | RIAVTYKKESDLAASETNNADFARLLALVNVACADAGIFSWKE |
| | KWEFEFWRPLSGVRDDGRPDHGDPFWLTLGAPATNTNDIPFKPP |
| | FPAYPSGHATFGGAAFQMVRRYYNGRVGTWKNDEPDTIAIDM |
| | MVSEELNGLSRDLRQPYDPSAPITDQPGIVRTRVVRHFSSAWEL |
| | MFENAISRLFLGVHWRFDAAAARDILIPTTTKDVYAKDENGATV |
| | FQNVEDVRYTTLGTREGHEGLLPIGGVPLGIGIADEIFESGLRPTP |
| | VERQPMGDGGEGVGGRKEEVMVVDGGMWEGEQVPAMDQAP |
| SEQ ID NO: 27 | MDPVTPIPLPQIDEPEEYNTNYILYWHHIGLELNRLTHTVGGPQT |
| Exserohilum | GPPISARALGMLQLAAHDAYFAINPSAEFSTELTPGAENAEYRLP |
| turcica | DLNGADDARQAVAGASLKMLTELYMKPVVQSNPNPGANISDR |
| | AYAQLALFIEQSSAEAPGGVDRASASFLFGEAVADVFFKLLFHPP |
| | GASQDGYHPTPGRYRFDDEPTHPVVLIPVDPNNPNGPKKPFRQY |
| | HGPFYGTTAKRFATQTEHILADPPGLRSAANETAEYDDSIRVAIA |
| | MGGAPGLNSTKRSPYQTAQGIFWAYDGSNLIGTPPRLYNQIVRR |
| | IAVTYKKEDDLANSEVNNADFARLLALVNVACADAGIFSWKEK |
| | WEFEFWRPLSGVRDDGRPDHGDPFWLTLGAPATNTNDIPFKPPF |
| | PAYPSGHATFGGAVFQMVRRYYNGRVGTWKDDEPDNIAIDMM |
| | VSEELNGLSRDLRQPYDPTAPITDQPGIVRTRIERHFNSAWEMML |
| | ENAISRIFLGVHWRFDAAAARDILIPTVTKDVYAIDNNGASVFQN |
| | AEDVRYSTRGTREGHDGLFPIGGVPLGIEIADEIFNAGLKPTPPEA |
| | QPMPQPPYVQKPPYGKESQSTMWEEEHAQAMDEAP |
| SEQ ID NO: 28 | MEPITPIPLPRIDEPEEYNTNYVLYWNHVGLELNRVTHTVGGPQT |
| Bipolaris maydis | GPPISARALGMLHLAIHDAYFAINPSADILTFLTPNAEDAAYRLP |
| | DLNGADDARQAVAGASLKMLSSLYMKPDMPPTNISDYAYAQL |
| | GLVLDRSAAEAPGGVDRASASFLFGEAVADVFFALLFHAPGASQ |
| | EGYQPTPGRYRFNDEPTHPVVLVPVDPNNPNGPKRPFRQYHAPF |
| | YGKTAKRFATQTDHILADPPGLRSAADESAEYDDSIRVAIAMGG |
| | AIGLNSTKRTPYQTVQGIFWAYDGSNLIGTPPRLYNQLVRHIAVT |
| | YKKEDDLANSEVNNADFARLFGLVNVACADAGIFSWKEKWEF |
| | EFWRPLSGVRDDGRPDHADPFWLTLGAPATNTNDIPFKPPFPAY |
| | PSGHATFGGAVFQMVRRYYNGRVGTWNDDEPDNIAIDMVVSE |
| | ELNGLSRDLRQPYDPTAPIEDQPGIVRTRVVRHFDSAWELMFEN |
| | AISRIFLGVHWRFDAAAARDILIPTTTKDVYAVDANGATVFQNV |
| | EDVRYSTKGTREGCEGLFPIGGVPLGIEIADEIFTSGLRPTPPEAQP |
| | APQEPPTVQKPIDQEVILGTQQEEAFVPAMNKAP |
| SEQ ID NO: 29 | MTDQRKLTAQRVREDANALAAGRIHPRHQANGDEQRYESANY |
| Synechococcus sp. | AMSFTKGLDHNTTTGLIEQSGDFEAFRSAIDNGFAEDFTRHVAV |
| | PRAEPRRKWEAPTAGTVYELQGPDPQAVTIPPAPALCSDELTFE |
| | MAEVYELALLRDLPFNAFVAGGGSAALADSTARLNSLAYAQDG |
| | FNRRPRTTNSSNQLDAQTVFRGSSPGVDQGPYLSQFMLIGNASPS |
| | EGITPEQGFINFGAQRIDQRVLEARQQDDYMMKWDDWHRVQQ |
| | GYEVRADRFDPCKSSGPGQAFTGQRRFIHTPRDLATYVHVDALY |
| | QAYLNACLLLLGNGTAFDPGFDLLSGGGEGLLHDPAGGQKVPL |
| | NAGGFALWGGPHVLSLVTEVATRGLKAVRYQKFNNHLRLRPE |
| | ALAARIEKAQEIESRFPTICGCFSEMASDLQQVVDLIRNHNQSLA |
| | GEATALLPMAFAEGSPMHPAYGAGHATVAGACVTILKAFFNTS |
| | ALFVKINDVAGFHSKQHILARLKCGDSVEAGAYQETDCGKRLEF |
| | ERCGSFHLIEGKYATFKPDGKTNQSCCPLTLEGELNKLAANISIG |
| | RNMAGVHYFSDYYDSLRMGEEIAIGILEEQALCYKTDPFVLSVP |
| | TFDGDVRRIGQR |
| SEQ ID NO: 30 | MATPCFAQAPAPTLRRSPRRLPLTPTSFLPRPFLPKPFPRSPRHPH |
| Chondrus crispus | PRLNAASARTSAAQPPDRAATPCATRQQQALELRRAAAQLAHD |
| | RAHPAHVSNGSEELHLHPKTGAPLLLANFTKGLPHERDTGLAAD |
| | AAHYDLFVAAVHSGLPDDIRAIPLGPQAAPGVRLPRSLRFRSGIA |
| | RSSHLFPHTDVRAWESMAAGNAFDLQGPDAQAVTMPPAPALAS |
| | PELALEMTENYWMALLRDVRFSQFAADPTVAAAMESMNATEW |
| | VALACGDASAVAEEERKRLRGPFTAQNVFRGNTQGDCKGPYIS |
| | QFLLSGSAGLGQVNSGADGYIQYGAMRMDQRVRVALEGLDYM |
| | TTWESWLDVQNGADLRGRELYEDGERFRFICTPRDLATYVHYD |
| | ALYQAYLNACIIMLNKKIPFDKGLPFQKDDDIDKQQGFALFGPP |
| | HILTLCTEVATRALKGIRFQKYNVHRRLRPEAVGGRVERYHHNC |
| | EDPLFADVKPLYDALDKDMLERVAAHNAEQNAKDDFGNSRFE |
| | DYSPTGSSGRTYLLPMAFPEGSPMHPAYGAGHAAVAGACTTILK |
| | AFFDHEHELDFAYVPTADGSKLEDVVDSLGEKLTVEGELNKVC |
| | SNISVGRNWAGVHYFTDYRESIILGEKIALGLLEEQKLTYAEEFS |
| | MTIPLFDGSTVTI |
| SEQ ID NO: 31 | MGIPADNLQSRAKASFDTRVAAAELALNRGVVPSFANGEELLY |
| Corallina pilulifera | RNPDPDNTDPSFIASFTKGLPHDDNGAIIDPDDFLAFVRAINSGDE |
| 3 | KEIADLTLGPARDPETGLPIWRSDLANSLELEVRGWENSSAGLTF |
| | DLEGPDAQSIAMPPAPVLTSPELVAEIAELYLMALGREIEFSEFDS |
| | PKNAEYIQFAIDQLNGLEWENTPAKLGDPPAEIRRRRGEVTVGN |
| | LFRGILPGSEVGPYLSQYIIVGSKQIGSATVGNKTLVSPNAADEFD |
| | GEIAYGSITISQRVRIATPGRDFMTDLKVFLDVQDAADFRGFESY |
| | EPGARLIRTIRDLATWVHFDALYEAYLNACLILLANGVPFDPNLP |
| | FQQEDKLDNQDVFVNFGSAHVLSLVTEVATRALKAVR |
| | YQKFNIHRRLRPEATGGLISVNKIAPQKGESIFPEVDLAVEELGDI |
| | LEKAEISNRKQNIADGDPDPDPSFLLPMAFAEGSPFHPSYGSGHA |
| | VVAGACVTILKAFFDSGIEIDQVFEVDKDEDKLVKSSFKGTLTV |
| | AGELNKLADNIAIGRNMAGVHYFSDQFESLLLGEQVAIGILEEQS |
| | LTYGENFFFNLPKFDGTTIQI |
| SEQ ID NO: 32 | MGIPADNLQSRAKASFDTRVAAAELALNRGVVPSFANGEELLY |
| Corallina pilulifera | RNPDPDNTDPSFIASFTKGLPHDDNGAIIDPDDFLAFVRAINSGDE |
| 2 | KEIADLTLGPARDPETGLPIWRSDLANSLELEVRGWENSSAGLTF |
| | DLEGPDAQSIAMPPAPVLTSPELVAEIAELYLMALGREIEFSEFDS |
| | PKNAEYIQFAIDQLNGLEWENTPAKLGDPPAEIRRRRGEVTVGN |
| | LFRGILPGSEVGPYLSQYIIVGSKQIGSATVGNKTLVSPNAADEFD |
| | GEIAYGSITISQRVRIATPGRDFMTDLKVFLDVQDAADFRGFESY |
| | EPGARLIRTIRDLATWVHFDALYEAYLNACLILLANGVPFDPNLP |
| | FQQEDKLDNQDVFVNFGSAHVLSLVTEVATRALKAVRYQKFNI |
| | HRRLRPEATGGLISVNKIAAQKGESIFPEVDLAVEELGDILEKAEI |
| | SNRKQNIADGDPDPDPSFLLPMAFAEGSPFHPSYGSGHAVVAGA |
| | CVTILKAFFDSGIEIDQVFEVDKDEDKLVKSSFKGTLTVAGELNK |
| | LADNIAIGRNMAGVHYFSDQFESLLLGEQVAIGILEEQSLTYGEN |
| | FFFNLPKFDGTTIQI |
| SEQ ID NO: 33 | MGIPADNLQSRAKASFDTRVAAAELALARGVVPSFANGEELLY |
| Corallina pilulifera | RDFLAFVRAINSGDEKEIADLTLGPARDPETGLPIWRSDLANSLE |
| 1 | LEVRGWENSSAGLTFDLEGPDAQSVAMPPAPVLMSPELIAEMAE |
| | LYLMALGRDIEFSEFESPKNAAFIRSAIERLNGLEWENTPAKLGD |
| | PPAEIRRRRGEVTVGNLFRGILPGSEVGPYLSQYIIVGSKQIGSAT |
| | VGNKTFVSPNAADEFDGEIAYGSITISQRVRIATPGRDFMTD |
| | LKVFLDVQDGADFRGFESYEPGARLIRTIRDLATWVHFDALYEA |
| | YLNACLILLANGVPFDPNLPFQQEDKLDNHDVFVNFGSAHVLSL |
| | VTEVATRALKAVRYQKFNIHRRLRPEATGGLISVNKKSFLAGSDI |
| | IFPEVSELVEELSSILDDVAESNEKQNRADGIVSPDKSFLLPMAFA |
| | EGSPFHPSYGSGHAVVAGACVTILKAFFDANFQIDKVFEVDTDE |
| | DKLVKSSFKGTLTVAGELNKLADNVAIGRNMAGVHYFSDQFES |
| | LLLGEQIAIGILEEQSLTYGENFFFNLPKFDGTTIQI |
| SEQ ID NO: 34 | MGIPADNLQSRAKASFDTRVAAAELALARGAVPSFANGEELLY |
| Corallina | RNSETGDPSFIGSFTKGLPHDDNGAIIDPDDFLAFVRAINSGDEKE |
| officinalis 2 | IAALTLGPARDPETGLPIWRSDLANSLDLEVRGWENSSAGLTFD |
| | LEGPDAQSVAMPPAPVLTSPELIAEMAELYLMALGRDIEFSEFDS |
| | PKNAAFIRSAIERLNGLEWENTPAKLGDPPAEIRRRRGEVTVGNL |
| | FRGILPGSEVGPYLSQFIIVGSKQIGSATVGNKTLVSPNAADEFDG |
| | EIAYGSITISQRVRIATPGRDFMTDLKVFLDVQDAADFRGFESYE |
| | PGARLIRTIRDLATWVHFDSLYEAYLNACLILLANGVPFDPNLPF |
| | QQEDKLDNQDVFVNFGSAHVLSLVTEVATRALKAVR |
| | YQKFNIHRRLRPEATGGLISVNKNAFLKSESVFPEVDVLVEEL- |
| | SSILDDSASSNEKQNIADGDVSPGKSFLLPMAFAEGSPFHPSYGS |
| | GHAVVAGACVTILKAFFDANFQIDQVFEVDTDEDKLVK--SF-- |
| | GPLTVAGELNKLADNVAIGRNMAGVHYFSDQFESLLLGEQIAIG |
| | ILEEQSLTYGENFFFNLPKFDGTTIQI |
| SEQ ID NO: 35 | MAGPTASGTARRNEAFDKRVAAATVARERVHPPHAANGEEYE |
| Neopyropia | YRATFDRHPAVAAAKAAAAGASREASNGDPGATDGGATAAGA |
| yezoensis | PPSSQSQVQGPSHIGSFTKGLQHNLSTGLLEDPAAFRAFVRAIDS |
| | GDPRDFVDVPIADRFTRPPTRGVRSWESASAGLAFDLQGPDAQA |
| | LTMPPAPRLGSAELVAEMIEVYAMALARDVPFATAEWAAA KP- |
| | IAAAVRELNATRWHGAAATDRELGLSPAAAARRRVTHFTPATV |
| | FRGVGRGVADGPYLSQFLLLGNAGVGGSSGAPTDGFITYGANR |
| | VDLRVPVATPGVDYLTDWESFVDVQNGADTRGTETYVPAADG |
| | TKTRFIATPRDLATYVHYDALYQAYLNAALWLLAEGVPFDAGL |
| | PWTAPDEVDHQQGFATFGGPHLLTLVTEVATRALKAVR |
| | FQKFNVHRRLRPEAVGGLLDRLPVAPGPLAPVAPLAEEAAETLR |
| | RVADANAAAAAGGSGAGAPVTHSVLLPMAFPEGSPMHPSYGA |
| | GHATVAGACVTVLKGWFDAGHALGTTAYEATPDGRCLREVST |
| | GGVALTVEGELNKLAANISIGRNWAGVHYYSDYIESVQLGEAV |
| | AIGILEEQKLTYGENWSMTVPLTDGTTVRI |
| SEQ ID NO: 36 | TRRQTALVKRIAAADLAANRHHPHHVRNADETTYKLPNHVC---- |
| Asparagopsis | SYISAFTKGLPHHLDGRLVSPHHFQLFIHAVRTGHVKDIAAIPLG |
| taxiformis 5 | PPHSSSFRSGIAKDAAVRGWESMAAGNTFDLQGPDAQAVTMPP |
| | CPRLNSHELVTEMTELYYMALLRDVPFTHFNKHHLVQSAVYDM |
| | NHTHWTKSNRCCLPRPERRRHRSKFTTDNIFRGVTPGDITG |
| | PYISQFLLVGTKGLADVHEETEGYVQYGATRIDQRVRTVQPKID |
| | YMTTWAAYLDVQNGADLRRHSAYAEPPNYRFICTPRDLATYVH |
| | YDALYQAYLNACIILLDINAPYDSGLPFTADDAVDKQQGFATFG |
| | APHVLTLVTEVATRALKAVRFQKYAVHRRLRPEAVGGLLEQYR |
| | RYGGGHSDLAYIIRPIRHLADSLSSDLMSMVAKENGRQNSLIRD |
| | NGHPRSHDEGSDDTHLLSMAYAEGSPMHPSYGAGHATVAGAC |
| | VTILKAFFDHTFKLPFAYVSSSDGRKLKTVKLSKKLTVEDELNK |
| | LAANISIGRSWAGVHYYSDYVESIRLGEEVAIGMLKEQKLTYSE |
| | KFTMTIPKFDGSVIEI |
| SEQ ID NO: 37 | TRRQTALVKRIAAADLAANRHHPLHVRNADETTYKLPNHVC---- |
| Asparagopsis | SYISAFTKGLPHHLDGRLVSPHDFLAFVRAINSGDEKEIAALTLG |
| taxiformis 2 | PARDPETGLPIWRSDLANSLDLEVRAWESMAAGNTFDLQGPDA |
| | QAVTMPPCPRLNSHELVTEMTELYYMALLRDVPFTHFNKHHLV |
| | QSAVHDMNHTHWTRSNRCCLPRPERRRHRSKFTTDNIFRGVTP |
| | GDITGPYISQFLLVGTKGLGDAHEETEGYVQYGATRIDQRVRTV |
| | EPKIDYMTTWAAYLDVQNGADLRRHSTYAKPPSYRFISTPRDLA |
| | TYVHYDALYQAYLNACIILLDINAPYDGGLPFTADDAVDKQQG |
| | FATFGAPHVLTLVTEVATRALKAVRFQKYAVHRRLRPEAVGGL |
| | LEQYRRYGGGHSDLAYIIRPIRHLADSLSSDLMFMVAKENGRQN |
| | SSITDNGHPRSHDEGSDDTHLLSMAYAEGSPMHPSYGAGHATV |
| | AGACVTILKAFFDHTFKLPFAYVSSSDGRKLKTVKLSRKLTVED |
| | ELNKLAANISIGRSWAGVHYYSDYVESIRLGEEVAIGMLKEQKL |
| | TYSEKFTMTIPKFDGSVIEI |
| SEQ ID NO: 38 | ISPRQQEAYDIRVAAATLALARDRKRKVFEPNGEEDRYFRPNSQI |
| Chondrus crispus 1 | LSYLGSYTKGLPHNRSTALVTSPNHFVRFVQAIDSGEPADFIKVP |
| | LGPRPALEFTTEEECIAANLFKSGIATRNDSFDPACLRAWESMGA |
| | GLVFDLEGPDAQAVGMPPAPTLDSDELVAEISELYWMALLRDV |
| | RFTDFTNPGPIRRAINTLNRTTWIRAARDPPDSLTPQERARLRGPF |
| | NPSSIFRGTLPGDDVGPYLSQFLLVGTEGIGGAQDRADGFISYGA |
| | HRIDQRVRHATKELDYMTSWEAFLDVQNGADVAGRDTFESGS |
| | DAFRFITTPRDLATYVHFDALYQAYLNACLILLDNKVKFDQGIP |
| | MGEPDFNGNQGIPFQDPDFKDHQRGFAHFGGPHILSLVTEVATR |
| | ALKAVRFQKFNTHRRLRPEAVGGLIERFNSNPDDPQFQDVKPLF |
| | EALDEDMLRRVASHNREQNERSDFGMPRADDFNPAGDTLETML |
| | LPMAFPEGSPMHPSYGAGHATVAGACVTVLKAFFQHDATLDFC |
| | YVPSDDGSRLDDASHTLNKKLTVEGELNKVCSNISIGRNWAGV |
| | HYFTDYIESILLGEQIALGILEEQMLTFPETFTMTVPLFSG |
| SEQ ID NO: 39 | ARRQEAFQTRVRAARLARDRDHQVFEPNGEEDRYFRPNSQIFSY |
| Chondrus crispus 3 | LGSYTKGLPHNRRTALITSPNHFVRFVRAIDSGEPADFIKVPLGPR |
| | PALQFESEEECRAANLFKSGIAGRNNTFPSSCLRAWESMGAGLV |
| | FDLEGPDAQAVGMPPAPTLDSDELVAEISEVYWMALLRDVRFT |
| | DFTNPGPIGRAINTLNKTAWIRAARDPPDSLTPQERARLRGPFNP |
| | SNIFRGTLPGDDVGPYLSQFLLVGTKGIGGAQDMADGFISYGAH |
| | RIDQRVRHARKELDYMTSWEAFLDVQNGADVTGRDTFESGSDA |
| | FRFITTPRDLATYVHFDALYQAYLNACLIMLDSGVRFDKGIPFGE |
| | PDFKDHQRGFAHFGGPHILSLVTEVATRALKAVRFQKFNTHRRL |
| | RPEAVGGLIERFNSNPDDPQFQDVKPLFEALDEDMMRRVARH |
| | NREQNQGSDFGMPRADDFNPAGDTLETMLLPMAFPEGSPMHPS |
| | YGAGHATVAGACVTVLKAFFQHDTELDFCFVPSDDGSRLVDAS |
| | HNMNKKLTVEGELNKVCSNISIGRNWAGVHYFTDYIESILLGEQI |
| | ALGILEEQMLTFPETFTMTVPLFSG |
| SEQ ID NO: 40 | QARKQEAFQTRVAAARLARDRDHQVFEPNGEEDRYFRPNSQILS |
| Chondrus crispus 2 | YLGSYTKGLPHNRRTALVTSPNHFVRFVRAIDSGEPADFIKVPLG |
| | PRPALEFESEEECRAANLFKSGIAGRNNTFPSSCLRAWESMGAG |
| | LVFDLEGPDAQAVGMPPAPTLDSDELVAEISEVYWMALLRDVR |
| | FTDFTNPGPIGRAVNTLNRTTWIRAARDPPDSLTPQERARLRGPF |
| | NPSNIFRGTLPGDDVGPYLSQFLLVGTEGIGGAQDRADGFISYGA |
| | HRIDQRVRHATKELDYMTSWEAFLDVQNGADVAGRDTFESGS |
| | DAFRFITTPRDLATYVHFDALYQAYLNACLIMLDSGVKFDKGIP |
| | FGEPDFKDHQQGFAHFGGPHILSLVTEVATRALKAVRFQKFNTH |
| | RRLRPEAVGGLIERFNSNPDDPQFQDVKPLFEALDEDMMRRVA |
| | RHNREQNQGSDFGMPRADDFNPAGDTLETMLLPMAFPEGSPMH |
| | PSYGAGHATVAGACVTVLKAFFQHDTELDFCFVPSNDGSRLVD |
| | ASHNMNKKLTVEGELNKVCSNISIGRNWAGVHYFTDYIESILLG |
| | EQIALGILEEQMLTFPETFTMTVPLFSG |
| SEQ ID NO: 41 | NRFTEAYNVRLEAATLAKNRPHPIHLANTEELRYRRPFDCEFPG |
| Gracilariopsis | QPSHIASYTKGLPHDPKTGLLIKPNDFQTFIRALDAGDEFSIREVP |
| chorda 1 | LGPQIPEPRFISGIASTPKADGSPTDVRAWESMAAGNTYGLEGPD |
| | AQAVTMPPAPALDSKELVYEISESYWMALVRDVPFTRFDDDVV |
| | RDAVRSLNQTEWIRFLAKGGPLPPSLSDAQRKRLRKPFTIDTVFR |
| | GVTKGDLEGPYLSQFMLIGNSGIANGNKIEDGFVQYGAHRVDQ |
| | KVRAVEDGRDYMTTFKSFVDVQNGADVRGREVYSDGFRFITTP |
| | RDLTTYVHFDALYQAYLNACLILLGMKVPFDKGLPFQLDDDVD |
| | KQQTFATFGGPHILSLVTEVATRALKAVRFQKYNVHRRLRPEAV |
| | GGLIEYYHNNPDSEIVAGVKPLYEALDKDMLNRVSMHNESQNK |
| | LCDGDVPRMSDFEDGESSATRLLPMAFPEGSPMHPSYGAGHAA |
| | VAGACVTVLKAFFDHGYELRMLDENGNMVPFAFVPTRDGQSL |
| | KNVISEYGNKPLTVEGELNKVCSNISIGRNWAGVHYFTDYRESIT |
| | LGEKIAIGILEEQKLTYGENFSMTVPLFDGTVYRI |
| SEQ ID NO: 42 | NRFTEAYNVRLEAATLAKNRPHPIHLANTEELRYRRPFDCEFPG |
| Gracilariopsis | QPSHIASYTKGLPHDPKTGLLIKPNDFQTFIRALDAGDEFSIREVP |
| chorda 3 | LGPQIPEPRFISGIASTPKADGSPTDVRAWESMAAGNTYDLEGPD |
| | AHAVTMPPAPALDSKELVYEISESYWMALVRDVPFTRFDDDVV |
| | RDAVRSLNQTEWIRFLAKGGPLPPSLSDAQRKRLRKPFTIDTVFR |
| | GVTKGDLEGPYLSQFMLIGNSGIANGNKIEDGFVQYGAHRVDQ |
| | KVRAVEDGRDYMTTFKSFVDVQNGADVRGREVYSDGFRFITTP |
| | RDLATYVHFDALYQAYLNACLILLGMKVPFDKGLPFQLDDDVD |
| | KQQTFATFGGPHILSLVTEVATRALKAVRFQKYNVHRRLRPEAV |
| | GGLIEYYHNNPDSEIVAGVKPLYEALDKDMLNRVSMHNESQNK |
| | LCDGDVPRMSDFEDGESSATRLLPMAFPEGSPMHPSYGAGHAA |
| | VAGACVTVLKAFFDHGHELRMPDENGNMVPFAFVPTRDGQSK |
| | NVISEYGNKPLTVEGELNKVCSNISIGRNWAGVHYFTDYRESITL |
| | GEKIAIGILEEQKLTYGENFSMTVPLFDGTVYRI |
| SEQ ID NO: 43 | NRFTEAYNVRLEAATLAKNRPHPIHLANTEELRYRRPFDCEFPG |
| Gracilariopsis | QPSHIASYTKGLPHDPKTGLLIKPNDFQTFIRALDAGDEFSIREVP |
| chorda 2 | LGPQIPEPRFISGIASTPKADGSPTDVRAWESMAAGNTYDLEGPD |
| | AHAVTMPPAPALDSKELVYEISESYWMALVRDVPFTRFDDDVV |
| | RDAVRSLNQTEWIRFLAKGGPLPPSLSDAQRKRLRKPFTIDTVFR |
| | GVTKGDLEGPYLSQFMLIGNSGIANGNKIEDGFVQYGAHRVDQ |
| | KVRAVEDGRDYMTTFKSFVDVQNGADVRGREVYSDGFRFITTP |
| | RDLTTYVHFDALYQAYLNACLILLGMKVPFDKGLPFQLDDDVD |
| | KQQTFATFGGPHILSLVTEVATRALKAVRFQKYNVHRRLRPEAV |
| | GGLIEYYHNNPDSEIVAGVKPLYEALDKDMLNRVSMHNESQNK |
| | LCDGDVPRMSDFEDGESSATRLLPMAFPEGSPMHPSYGAGHAA |
| | VAGACVTVLKAFFDHGYELRMPDENGNMVPFAFVPTRDGQSL |
| | KNVISEYGNKPLTVEGELNKVCSNISIGRNWAGVHYFTDYRESIT |
| | LGEKIAIGILEEQKLTYGENFSMTVPLFDGTVYRI |
| SEQ ID NO: 44 | SRDNKSYGVREQAALLAKSRPHPIHVANAEELRYRIPFDGSAIQG |
| Gracilaria | MPSHIASYTKGLPHDLETGVLVNPNDFQTFIRAMDSGDKFSIKEI |
| domingensis | PLGPQIPEPRFTSGIAGPGTDVRAWESMGAGNTFDLEGPDAQSV |
| | TMPPAPTLDSDELVTEITECYWMALLRDISFSEFSTNNLVCEAVE |
| | SLNKTRWVELSKHGATVPASLTVPERRRLRGPFTSETVFRGTTR |
| | GDDVGPYLSQFLLVGNSGIAGGNKISDGQIQYGAHRVDQRVRV |
| | AEKGRDFMTTWQSFMDVQNGADVRGREVYDNDVPFRFIATPR |
| | DLATYVHFDALYQAYLNACLILLGMKVPFDKGLPFQLDDDVDR |
| | QQTFATFGAPHILSLVTEVATRALKAVRFQKFNVHRRLRPEAVG |
| | GLIERFLEDPASDLFSGVQNLCEQLDESLVKTVRKHNKKQNEK- |
| | SDNGVPRNDDKGAGRTMLMPMAFPEGSPMHPSYGAGHATVAG |
| | ACVTVLKAFFDHGYELRMKDDEGKMVPFAFVANEDGSELVNEI |
| | EKYGNKALTVEGELNKVCSNICIGRNWAGVHYFSDYRESIEIGE |
| | KIAIGVLEEQKLTYGENFSMSVPLFDGSVYRI |
| SEQ ID NO: 45 | RFTKAYAVREQAALMAKSRPHPLHVSNNEELRYRIPTSGEPSHI |
| Gracilaria changii | GSYTKGLPHDRNTGVLSNPDDFQSFIRAMDAGDQFSIRDVPLGP- |
| | QNEQQAFQSGIAKGVSARAWESMAAGLTYDLEGPDAQSVTMPP |
| | APALDSDELLTEVTELYWMALLRDVPFSEFNGDKNNDIQEAVD |
| | SLNSTPWVQIAKNGVVPTTLTDAERVRLRGPFTVDNVFRGTTRG |
| | DRDGPYLSQFMLIGNTGIGNGNRIEDGFIRYGAHRVDQRVRVAE |
| | MGRDFMTTWSAFVDVQNGADVRGREVYNNDVRYRFISTPRDL |
| | ATYVHFDALYQAYLNACLILLGMRVPFDKGLPFQREDDIDHQQ |
| | TFATFGAPHILSLVTEVATRALKAVRFQKFNVHRRLRPEAVGGL |
| | LHRYMQETTNDLYRGAKQLAENLDANLLDKVKRHNAKQNMK- |
| | SDGDAPRANDLGECEDADEITLLPMAFPEGSPMHPSYGAGHAT |
| | VAGACVTILKAFFDHGYELRMLDEDGETMIPFAFVPTADGSGLE |
| | NRIGEYRNLTALTVEGELNKVCSNICIGRNWGGVHYFSDYRESI |
| | RVGEQVAIGLLEEQKLTYGENFSMTVPLFDGSVHRI |
| SEQ ID NO: 46 | ATPCATRQQQALELRRAAAQLAHDRAHPAHVSNGSEELHLHPK |
| Chondrus crispus 4 | TGAPLLLANFTKGLPHERDTGLAADAAHYDLFVAAVHSGLPDD |
| | IRAIPLGPQAAPGVRLPRSLRFRSGIARSSHLFPHTDVRAWESMA |
| | AGNAFDLQGPDAQAVTMPPAPALASPELALEMTENYWMALLR |
| | DVRFSQFAADPTVAAAMESMNATEWVALACGDASAVAEEERK |
| | RLRGPFTAQNVFRGNTQGDCKGPYISQFLLSGSAGLGQVNSGAD |
| | GYIQYGAMRMDQRVRVALEGLDYMTTWESWLDVQNGADLRG |
| | RELYEDGERFRFICTPRDLATYVHYDALYQAYLNACIIMLNKKIP |
| | FDKGLPFQKDDDIDKQQGFALFGPPHILTLCTEVATRALKGIRFQ |
| | KYNVHRRLRPEAVGGRVERYHHNCEDPLFADVKPLYDALDKD |
| | MLERVAAHNAEQNAKDDFGNSRFEDYSPTGSSGRTYLLPMAFP |
| | EGSPMHPAYGAGHAAVAGACTTILKAFFDHEHELDFAYVPTAD |
| | GSKLEDVVDSLGEKLTVEGELNKVCSNISVGRNWAGVHYFTDY |
| | RESIILGEKIALGLLEEQKLTYAEEFSMTIPLFDGSTVTI |
| SEQ ID NO: 47 | NRAEVAFKVRVSAAELARARGSPAHLSNNSESRFRNPDGTRSLL |
| Asparagopsis | ANFTKGLPHIKETALVESAIDYDRFVRAIDSGDPRDFADLPLGPQ |
| taxiformis 6 | GVEPRFTSGIASDPEVGTRAWESGGAGLVFDLEGPDAQAVTMPP |
| | APELDSDELVAEVTECYWMSLLRDVPFPTFESNSHIQAAAESINN |
| | TQWIKFKNNPPAHLTAAERSRLRGPVTTANVFRGITPGDEVGPY |
| | LSQFLLVGTTGIANGNEVGDGFIQYGGMRMDQRVRVAKPHIDY |
| | MTTFGAYLDVQNAANVSGRELYKEEEPRFRFIHTPRDLATYVHF |
| | DALYQAYLNACIILLDIGAPFDSGIPFQLDNDIDKQQGFATFGGP |
| | HILSLVTEVATRALKAVRFQKFNVHRRLRPEAIGARVDRYCAT- |
| | KAPEFAGAAKLSEALDKELLQKVHDHNKKQNLLSDRGNPRAN |
| | DFNPDGEVSEGNLLMPMAFPEGSPMHPAYGAGHATVAGACVT |
| | VLKAFFDGGYRLPFCYITDEDGTGLQAVEIDEPLTVDGELNKICS |
| | NISIGRNWAGVHYFTDYIESIRIGEEIAIGILQEQKLTFSENFSMTL |
| | NKFDGSTIRI |
| SEQ ID NO: 48 | NRAEVAFKVRVSAAELARARGSPTHLSNNSESRFRNPDGTRSLL |
| Asparagopsis | ANFTKGLPHTKETALVVSAIDYDSFVRAIDSGDPRDFADLPLGPQ |
| taxiformis 3 | GVEPRFTSGIASDPEVGTRAWESGGAGLVFDLEGPDAQAVTMPP |
| | APELDSDELVTEVTECYWMSLLRDVPFPTFDSDPHIQSAAESINN |
| | TQWIQFKNNPPAHLTAAERSRLRGPITTANVFRGITPGDEVGPYL |
| | SQFLLVGTTGIANGNEVGDGFIQYGGMRMDQRVRVAKSHIDYM |
| | TTFGAYLDVQNAANVSGRELYNEEEPRFRFIHTPRDLATYVHFD |
| | ALYQAYLNACIILLDIGAPFDSGIPFQLDNDIDKQQGFATFGGPHI |
| | LSLVTEVATRALKAVRFQKFNVHRRLRPEAIGARVDRYCATKA |
| | PEFAGAAKLSEALDKKLLQKVHDHNKKQNLLSDRGNPRANDEN |
| | PDGDVSKGNLLMPMAFPEGSPMHPAYGAGHATVAGACVTVLK |
| | AFFDGGYRLPFCYITDENGTGLKAVEIDEPLTVDGELNKICSNISI |
| | GRNWAGVHYFTDYIESIRIGEEIAIGILQEQKLTFSENFSMTLNKF |
| | DGSTIRI |
| SEQ ID NO: 49 | EERRQNALEIRIQAAKLAKKRDHPTHKANGDEDRYPETLIGSFT |
| Asparagopsis | KGLPHEKETGLLSNPADFADFVRAINTGAIKDMRRLKLGIDEDP |
| taxiformis 4 | RFISGIASKGDKHPFADTRAWESMAAGLTYDLEGPDAQAVTMP |
| | PAPKLDSDELVTEITESYWMALLRDVPFTEFERDGNTAAAAASIS |
| | RTRWVQYNEQPSQRPSTLTDEEIARLRGPYTKKNVFRGVTNGEN |
| | VGPYLSQFLLVGTKGIGDAQQVSDGYVQYGGMRMDQRVRVAV |
| | PKRDYMTTWASWLDVQNAGDLRGREIYDDDTPFRFITTPRDLA |
| | TWVHFDALYQAYLNACIILLDIKAPFDPHIPFQADDDVDKQQGF |
| | ATFGGPHILSLCTEVATRALKAVRFQKYNLHRRLRPEAIGGLVE |
| | RFKKTNGDPKFAPVKKLVNDLDGDMLRRVEQHNCEQNKLSDD |
| | GHARREDYSPEGESSQSYLLPMAFPEGSPMHPSYGAGHATVAG |
| | ACVTVLKAFFDHEYELDFCYVPTTDGKRLEKVNINEKLTVEGEL |
| | NKLCANISIGRNWAGVHYYSDYFESIKVGEEIAIGILQEQKLTYG |
| | EDFFMTLPKFDGEKIRI |
| SEQ ID NO: 50 | EERRQNALDIRIQAARLAKKRDHPTHEANGDEDRYPETLIGSFT |
| Asparagopsis | KGLPHEKETGLLSNPAAWESMAAGLTYDLEGPDAQAVTMPPAP |
| taxiformis 1 | KLDSDELVTEITESYWMALIRDVPFTEFERDGNTAAAAASISRTR |
| | WVQFNEQPSQRPSTLTDEEIARLRGPYTKKNVFRGVTIGENVG |
| | PYLSQFLLVGTKGIGDVQQISDGYIQYGSMRMDQRVRVAAPKR |
| | DYMTTWASWLDVQNAGDLRGREIYDDDTPFRFITTPRDLATWV |
| | HFDALYQAYLNACIILLDIKAPFDPHIPFQADDDVDKQQGFATFG |
| | GPHILSLCTEVATRALKAVRFQKYNVHRRLRPEAIGGLVERFKK |
| | TNGDAKFAPVKKLVNDLDGDMLRRIEQHNCEQNKLSDDGHAR |
| | RDDYSPEGESNQTYLLPMAFPEGSPMHPSYGAGHATVAGACVT |
| | VLKAFFDHEYELDFCYVPTTDGKRLEKVNINEKLTVEGELNKLC |
| | ANICIGRNWAGVHYYSDYFESIKVGEEIAIGILQEQKLTYGEDFF |
| | MTLSKFDGEKIRI |

### Host Cell Microorganisms

The present disclosure provides peroxidases and related methods for producing a plurality of small halogenated organic compounds useful, e.g., for the reduction of methane in the rumen of agricultural animals. In an embodiment, the peroxidases (VHPOs) are heterologously produced in a host cell microorganism. In an embodiment, producing a peroxidase (VHPO) in a host cell microorganism results in an increase in the total quantity of peroxidase (VHPO), e.g., compared with a reference standard. As used herein, producing a peroxidase (VHPO) in a host cell microorganism includes the expression, translation, and/or secretion of the peroxidase (e.g., VHPO).

A host cell microorganism suitable for use in the present disclosure is capable of producing a peroxidase (VHPO) described herein. In an embodiment, the host cell microorganism naturally produces a peroxidase (VHPO), e.g., expresses an endogenous peroxidase (VHPO). In an embodiment, the host cell microorganism produces an exogenous peroxidase (VHPO). In an embodiment, the host cell microorganism is genetically modified to produce a peroxidase (VHPO), e.g., to express a heterologous peroxidase (VHPO). In such embodiments, a nucleic acid encoding a heterologous peroxidase (VHPO) is introduced to the host cell microorganism using standard methods known in the art, e.g., by electroporation, transfection, or transduction. The heterologous peroxidase (VHPO) may be a peroxidase (VHPO) that is naturally produced in a different microorganism or may be a modified a peroxidase (VHPO) comprising a different amino acid sequence or different function and/or activity, e.g., increased or decreased activity, from that of the corresponding naturally occurring a peroxidase (VHPO).

The host cell microorganism can be a fungus (e.g. a yeast), a bacterium, a protozoan, an archaeon, a synthetic organism or a semi-synthetic organism that produces one or more proteins, e.g., one or more enzymes, such as one or more peroxidases (e.g., VHPOs). In an embodiment, the host cell microorganism is a fungus. In an embodiment, the host cell microorganism is a bacterium. In an embodiment, the host cell microorganism is a protozoan. In an embodiment, the host cell microorganism is an archaeon. Exemplary host cell microorganisms include Pichia pastoris, Aspergillus niger, Saccharomyces cerevisiae, Escherichia coli, and Synechococcus sp. PCC11901.

Pichia pastoris is a highly successful heterologous expression system. The yeast Pichia pastoris has been used in biotechnology as an expression system for protein and enzyme production, as it has shown to result in increased protein production over many native systems. Pichia can reach high cell density growing on a defined medium resulting in upwards of 250 g/L wet cell density of produced protein (e.g., secreted protein), and has excellent scalability (e.g., scalability of up to ˜200,000 liters of cell culture). Appropriate folding and secretion are two of the main advantages of the P. pastoris system. The system is also often used for the production of complex eukaryotic proteins, and it has been successfully used to produce fungal enzymes, including beta-glucosidase, lignocellulosic enzymes, and oxidases from macroalgae. While P. pastoris expression systems may be facile for use on both experimental and industrial scales, the production of recombinant proteins in P. pastoris often requires an optimization process to reach maximum production of the proteins of interest. It is widely known that many conditions must be tested to yield the desired results, including optimization of culture medium components, growth temperature, and protein and nucleic acid sequences. P. pastoris cells can also be converted into cell factories for large-scale production.

Aspergillus niger is a filamentous mold initially developed to produce citric acid and now fundamental for producing a diverse range of proteins, enzymes, and secondary metabolites. A. niger is now being increasingly used as an alternative biologtems. The products include proteins including enzymes, but also pharmaceuticals which used for human and animal health.

Saccharomyces cerevisiae is a commonly utilized host cell microorganism for improving heterologous production of proteins, e.g., enzymes. In addition to the mass-production potential of baker's yeast, extensive research exists on approaches to concentrate products in organelles, such as peroxisomes, within the cell, which has significant advantages in biomanufacturing. S. cerevisiae's peroxisomes have been engineered for equivalent reactions, and the mass-production of this yeast is well-established in the food industry (e.g., wine, beer, bread). Intracellular compartmentalization can provide optimal conditions for desired enzymatic reactions by increasing the metabolic flux and reducing the metabolic crosstalk, making it a powerful strategy to isolate toxic products away from the hosts' cytosol, thereby limiting cytotoxicity, improving growth, and increasing product titer.

Cyanobacteria are prokaryotic oxyphotoautotrophs able to convert CO₂ and inorganic sources of nitrogen, phosphorus, and microelements into biomass. Synechococcus PCC11901, a robust marine cyanobacterial strain established for high biomass production on an industrial scale. PCC11901 can be cultivated in seawater unsuitable for agricultural use or direct human consumption; other promising features include: (i) it grows at high light intensities and in a wide range of salinities, (ii) it accumulates up to ≈33 g dry cell weight/L, (iii) genome and genetic tools are already available. PCC 11901 interestingly lacks a VHPO, but can heterologously express the CiVHPO enzyme. In addition, there is a large body of literature on Synechococcus sp. ecology, genetics, cell biology, and biotechnology applications that can be directly applied to PCC11901. Strains of interest have been engineered for fast-growth, solvent tolerance, product biosynthesis and accumulation, and biocontainment.

In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to a peroxidase (e.g., the VHPO) described herein, or a functional fragment thereof, e.g., at least 80%, at least 85%, at least 90%, at least 91%, at least 92%, at least 93%, at least 94%, at least 95%, at least 96%, at least 97%, at least 98%, or at least 99% identity to a peroxidase (e.g., the VHPO) described herein, or a functional fragment thereof.

In an embodiment, the peroxidase (e.g., the VHPO) comprises an amino acid sequence listed in Table 2. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to an amino acid sequence listed in Table 2. In an embodiment, the peroxidase (e.g., the VHPO) comprises the amino acid sequence of SEQ ID NO: 1. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 1. In an embodiment, the peroxidase (e.g., the VHPO) comprises the amino acid sequence of SEQ ID NO: 2. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 2. In an embodiment, the peroxidase (e.g., the VHPO) comprises the amino acid sequence of SEQ ID NO: 3. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 3. In an embodiment, the peroxidase (e.g., the VHPO) comprises the amino acid sequence of SEQ ID NO: 1. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 1. In an embodiment, the peroxidase (e.g., the VHPO) comprises the amino acid sequence of SEQ ID NO: 4. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 4. In an embodiment, the peroxidase (e.g., the VHPO) comprises the amino acid sequence of SEQ ID NO: 5. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 5. In an embodiment, the peroxidase (e.g., the VHPO) comprises the amino acid sequence of SEQ ID NO: 6. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 6. In an embodiment, the peroxidase (e.g., the VHPO) comprises the amino acid sequence of SEQ ID NO: 7. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 7. In an embodiment, the peroxidase (e.g., the VHPO) comprises the amino acid sequence of SEQ ID NO: 8. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 8. In an embodiment, the peroxidase (e.g., the VHPO) comprises the amino acid sequence of SEQ ID NO: 9. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 9. In an embodiment, the peroxidase (e.g., the VHPO) comprises the amino acid sequence of SEQ ID NO: 10. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 10. In an embodiment, the peroxidase (e.g., the VHPO) comprises the amino acid sequence of SEQ ID NO: 11. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 11. In an embodiment, the peroxidase (e.g., the VHPO) comprises the amino acid sequence of SEQ ID NO: 12. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 12. In an embodiment, the peroxidase (e.g., the VHPO) comprises the amino acid sequence of SEQ ID NO: 13. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 13. In an embodiment, the peroxidase (e.g., the VHPO) comprises the amino acid sequence of SEQ ID NO: 14. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 14. In an embodiment, the peroxidase (e.g., the VHPO) comprises the amino acid sequence of SEQ ID NO: 15. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 15. In an embodiment, the peroxidase (e.g., the VHPO) comprises the amino acid sequence of SEQ ID NO: 16. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 16. In an embodiment, the peroxidase (e.g., the VHPO) comprises the amino acid sequence of SEQ ID NO: 17. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 17. In an embodiment, the peroxidase (e.g., the VHPO) comprises the amino acid sequence of SEQ ID NO: 18. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 18. In an embodiment, the peroxidase (e.g., the VHPO) comprises the amino acid sequence of SEQ ID NO: 19. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 19. In an embodiment, the peroxidase (e.g., the VHPO) comprises the amino acid sequence of SEQ ID NO: 20. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 20. In an embodiment, the peroxidase (e.g., the VHPO) comprises the amino acid sequence of SEQ ID NO: 21. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 21. In an embodiment, the peroxidase (e.g., the VHPO) comprises the amino acid sequence of SEQ ID NO: 22. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 22. In an embodiment, the peroxidase (e.g., the VHPO) comprises the amino acid sequence of SEQ ID NO: 23. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 23. In an embodiment, the peroxidase (e.g., the VHPO) comprises the amino acid sequence of SEQ ID NO: 24. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 24. In an embodiment, the peroxidase (e.g., the VHPO) comprises the amino acid sequence of SEQ ID NO: 25. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 25. In an embodiment, the peroxidase (e.g., the VHPO) comprises the amino acid sequence of SEQ ID NO: 26. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 26. In an embodiment, the peroxidase (e.g., the VHPO) comprises the amino acid sequence of SEQ ID NO: 27. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 27. In an embodiment, the peroxidase (e.g., the VHPO) comprises the amino acid sequence of SEQ ID NO: 28. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 28. In an embodiment, the peroxidase (e.g., the VHPO) comprises the amino acid sequence of SEQ ID NO: 29. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 29. In an embodiment, the peroxidase (e.g., the VHPO) comprises the amino acid sequence of SEQ ID NO: 30. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 30. In an embodiment, the peroxidase (e.g., the VHPO) comprises the amino acid sequence of SEQ ID NO: 31. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 31. In an embodiment, the peroxidase (e.g., the VHPO) comprises the amino acid sequence of SEQ ID NO: 32. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 32. In an embodiment, the peroxidase (e.g., the VHPO) comprises the amino acid sequence of SEQ ID NO: 33. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 33. In an embodiment, the peroxidase (e.g., the VHPO) comprises the amino acid sequence of SEQ ID NO: 34. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 34. In an embodiment, the peroxidase (e.g., the VHPO) comprises the amino acid sequence of SEQ ID NO: 35. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 35. In an embodiment, the peroxidase (e.g., the VHPO) comprises the amino acid sequence of SEQ ID NO: 36. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 36. In an embodiment, the peroxidase (e.g., the VHPO) comprises the amino acid sequence of SEQ ID NO: 37. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 37. In an embodiment, the peroxidase (e.g., the VHPO) comprises the amino acid sequence of SEQ ID NO: 38. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 38. In an embodiment, the peroxidase (e.g., the VHPO) comprises the amino acid sequence of SEQ ID NO: 39. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 39. In an embodiment, the peroxidase (e.g., the VHPO) comprises the amino acid sequence of SEQ ID NO: 40. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 40. In an embodiment, the peroxidase (e.g., the VHPO) comprises the amino acid sequence of SEQ ID NO: 41. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 41. In an embodiment, the peroxidase (e.g., the VHPO) comprises the amino acid sequence of SEQ ID NO: 42. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 42. In an embodiment, the peroxidase (e.g., the VHPO) comprises the amino acid sequence of SEQ ID NO: 43. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 43. In an embodiment, the peroxidase (e.g., the VHPO) comprises the amino acid sequence of SEQ ID NO: 44. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 44. In an embodiment, the peroxidase (e.g., the VHPO) comprises the amino acid sequence of SEQ ID NO: 45. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 45. In an embodiment, the peroxidase (e.g., the VHPO) comprises the amino acid sequence of SEQ ID NO: 46. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 46. In an embodiment, the peroxidase (e.g., the VHPO) comprises the amino acid sequence of SEQ ID NO: 47. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 47. In an embodiment, the peroxidase (e.g., the VHPO) comprises the amino acid sequence of SEQ ID NO: 48. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 48. In an embodiment, the peroxidase (e.g., the VHPO) comprises the amino acid sequence of SEQ ID NO: 49. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 49. In an embodiment, the peroxidase (e.g., the VHPO) comprises the amino acid sequence of SEQ ID NO: 50. In an embodiment, the peroxidase (e.g., the VHPO) comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO: 50.

In an embodiment, the peroxidase (e.g., the VHPO) comprises an amino acid sequence with 1, 2, 3, 4, 5, 6, 7, 8, 9, 10, 11, 12, 13, 14, 15, or more mutations relative to a peroxidase (e.g., the VHPO) described herein, or a functional fragment thereof. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to a peroxidase (e.g., the VHPO) listed in Table 2. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 1. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 2. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 3. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 4. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 5. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 6. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 7. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 8. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 9. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 10. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 11. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 12. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 13. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 14. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 15. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 16. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 17. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 18. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 19. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 20. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 21. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 22. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 23. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 24. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 25. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 26. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 27. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 28. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 29. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 30. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 31. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 32. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 33. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 34. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 35. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 36. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 37. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 38. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 39. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 40. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 41. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 42. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 43. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 44. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 45. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 46. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 47. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 48. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 49. In an embodiment, the peroxidase (e.g., the VHPO) comprises an amino acid sequence with 1, 2, 3, 4, 5, 6, 7, 8, 9, 10, 11, 12, 13, 14, 15, or more mutations relative to the amino acid sequence of SEQ ID NO: 50.

Percent identity in the context of two or more amino acid or nucleic acid sequences, refers to two or more sequences that are the same, e.g, conserved. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 5 amino acids, 10 amino acids, 15 amino acids, 20 amino acids, 25 amino acids, 30 amino acids, 40 amino acids, 50 amino acids, 75 amino acids, 100 amino acids, or 150 amino acids in length. The identity may exist over a region that is between about 10 amino acids to about 100 amino acids, or about 50 amino acids to about 250 amino acids, or about 200 amino acids to about 500 amino acids in length.

For sequence comparison, one sequence typically acts as a reference sequence to which one or more test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

Functional variants may comprise one or more mutations, such that the variant retains some level of activity, e.g., peroxidase (e.g., a VHPO) described herein produced by the microorganism from which the enzyme originates from. In an embodiment, the functional variant has at least 10%, at least 20%, at least 30%, at least 40%, at least 50%, at least 60%, at least 70%, at least 75%, at least 80%, at least 85%, at least 90%, at least 95%, or at least 99% (e.g., at least 80%, at least 85%, at least 90%, at least 95%, or at least 99%) of the peroxidase (e.g., the VHPO) as the corresponding naturally occurring peroxidase (e.g., VHPO). In embodiments, the functional variant has at least 200%, at least 300%, at least 400%, at least 500%, at least 1000% or more of the peroxidase (e.g., VHPO) as the corresponding naturally occurring peroxidase (e.g., VHPO). Peroxidase activity can be tested using the functional assays known in the art. For example, if the peroxidase (e.g., VHPO) is a VBPO, then functional assays that measure consumption of bromide activity can be performed. In addition, functional assays may be coupled with each other; for example, if the peroxidase is a VBPO, then the VBPO activity may be coupled with consumption of a peroxide-containing compound, such as H₂O₂ or peracetic acid (PAA). Other exemplary assays include MCD and APF assays.

The mutations present in a functional variant include amino acid substitutions, additions, and deletions. Mutations can be introduced by standard techniques known in the art, such as site-directed mutagenesis and PCR-mediated mutagenesis. Mutagenesis can also be achieved through using CRISPR (Clustered regularly-interspaced short palindromic repeats)/Cas systems. The CRISPR/Cas system is naturally found in bacteria and archaea, and has been modified for use in gene editing (silencing, enhancing or mutating specific genes) in eukaryotes such as mice or primates (Wiedenheft et al. (2012) Nature 482: 331-8). This is accomplished by introducing into the cell a plasmid containing a specifically designed CRISPR and one or more appropriate Cas.

The mutation may be a conservative amino acid substitution, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the peroxidase (e.g., VHPO) can be replaced with other amino acids from the same side chain family, and the resultant peroxidase (e.g., VHPO) activity comparable (e.g., at least 80%, 85%, 90%, 95%, or 99% of the peroxidase (e.g., VHPO) activity) to that of the wild-type peroxidase (e.g., VHPO). Alternatively, the mutation may be an amino acid substitution in which an amino acid residue is replaced with an amino acid residue having a different side chain.

Such mutations may alter or affect various enzymatic characteristics of the peroxidase (e.g., VHPO). For example, such mutations may alter or affect the activity, e.g., the peroxidase activity, thermostability, optimal pH for reaction, enzyme kinetics, or substrate recognition of the enzyme, e.g., the peroxidase (e.g., VHPO). In some embodiments, a mutation results in an increase of the peroxidase (e.g., VHPO) activity of the variant in comparison to the peroxidase (e.g., VHPO) activity of the wild-type peroxidase (e.g., VHPO). In some embodiments, a mutation results in an increase or decrease in the thermostability of the variant in comparison to a wild-type peroxidase (e.g., VHPO). In an embodiment, a mutation changes the pH range at which the variant optimally performs the peroxidase reaction in comparison to wild-type peroxidase (e.g., VHPO). In an embodiment, a mutation results in the increase or decrease in the kinetics of the peroxidase (e.g., VHPO) (e.g., k_{cat}, K_{M}, k_{cat}/K_{M}, or K_{D}) in comparison to wild-type type peroxidase (e.g., VHPO). In an embodiment, a mutation results in an increase or decreases in the ability of the peroxidase (e.g., VHPO) to recognize or bind to the substrate in comparison to wild-type peroxidase (e.g., VHPO). In an embodiment, a mutation results in an increase or decrease in the selectivity of the peroxidase (e.g., VHPO) for a small organic compound compared to wild-type peroxidase (e.g., VHPO).

In addition, such mutations may alter or affect the stability or expression level of the peroxidase (e.g., VHPO). For example, such mutations may increase the stability of the peroxidase (e.g., VHPO) at a certain condition (e.g., temperature or pH) or increase the expression level of the peroxidase (e.g., VHPO) in a certain host cell microorganism. In an embodiment, a mutation results in an increase of peroxidase (e.g., VHPO) stability compared to the wild-type sequence. In an embodiment, a mutation results in an increase of the expression level of the peroxidase (e.g., VHPO) compared to the wild-type sequence.

In an embodiment, the peroxidase sequence contains an N-terminal signal sequence. In an embodiment, the peroxidase sequence contains an N-terminal protein purification tag (e.g., a cleavable or non-cleavable protein purification tag). In an embodiment, the peroxidase sequence contains a C-terminal protein purification tag (e.g., a cleavable or non-cleavable protein purification tag).

### Cell Culture and Protein Expression

The host cell microorganism that can produce a protein, e.g., peroxidase (e.g., VHPO) can be in a cell culture. A cell culture comprises one or more cells in a cell culture medium. The cell culture medium can be an aqueous cell culture medium comprising components that support cell maintenance, cell viability, cell growth, and/or cell proliferation. Cell culture media typically comprises physiological salts, e.g., ammonium salt, phosphate salt, potassium salt, magnesium salt, calcium salt, iron salt, manganese salt, zinc salt, or cobalt salt; amino acids; water, and optionally, a carbon source. In an embodiment, a cell culture media suitable for growing a host cell microorganism described herein comprises an ammonium salt, e.g., ammonium sulfate and/or ammonium hydroxide; a potassium salt, e.g., potassium hydroxide; a calcium salt, e.g., calcium chloride; a magnesium salt, e.g., magnesium sulfate; a manganese salt, e.g., manganese sulfate; an iron salt, e.g., iron sulfate; a zinc salt, e.g., zinc sulfate, a cobalt salt, e.g., cobalt chloride, phthalic acid; lactose; antibiotics, e.g., ACETOBAN®; and a carbon source, e.g., glycerol or carbon dioxide.

The host cell microorganism or cell culture is contacted with, e.g., fed, a carbon source, such as a sugar, to support the growth or proliferation of the host cell microorganism. In an embodiment, the host cell microorganism or cell culture is contacted with, e.g., fed, glucose. As the host cell microorganism proliferates in culture, the cell culture can be transferred from one container, e.g., a cell culture container, to a larger container to allow and encourage the host cell microorganism to continue to proliferate. For example, the host cell microorganism is contacted with sugar in a first container under suitable conditions, as described herein, such that the host cell microorganism proliferates. The proliferation can be monitored, and once a desired level of growth, e.g., a specific growth phase, or a desired level of proliferation, e.g., as measured by turbidity of the culture or by cell number, the host cell microorganism can be transferred to a second container, where the second container is larger, e.g., by volume, than the first container. Transferring the microorganism to the larger second container allows and encourages the microorganism to continue to proliferate. In embodiments, the microorganism is transferred once, e.g., from a first container to a larger second container. In embodiments, the host cell microorganism is transferred more than once, e.g., two, three, four, five, six, seven, eight, nine, or ten times, wherein for each transfer, the host cell microorganism is transferred into a container that is larger than the container from which the host cell microorganism was transferred from.

Containers suitable for transferring and culturing the host cell microorganisms described herein include any cell culture container known in the art. Examples of suitable containers include, but are not limited to, a cell culture flask, a roller bottle, a bioreactor, or a tank. Other cell culture conditions appropriate for maintaining cell viability or promoting cell proliferation are known in the art. Cell culture conditions for consideration include pH, temperature, oxygen levels, and movement. The pH of the cell culture, e.g., the media, is generally at physiological pH, e.g., between pH 4-8, or between pH 5-7, e.g., at pH 5, pH 6, or pH 7. The temperature for growth of a host cell microorganism producing a peroxidase (e.g., VHPO) is generally between 2° and 40° C., e.g., 30° C. In some embodiments, a particular strain of the host cell microorganism may show enhanced proliferation or enzyme production at an elevated temperature, e.g., 32 or 37° C., or at a lower temperature, e.g., 27° C., respectively. The cell culture may be stationary or may use movement to promote maintenance or proliferation. For example, the cell culture may be rolled, shaken, or agitated to enhance cell proliferation. The cell culture conditions disclosed herein are merely exemplary and should not be construed as limiting. Varying cell culture conditions from those explicitly listed herein may be envisioned or experimentally determined and may depend on the species or strain of host cell microorganism used. Cell culture conditions sufficient for proliferation of the host cell microorganism that can produce a peroxidase (e.g., VHPO) result in an increase in the cell number of a culture of the host cell microorganism. Cell culture conditions sufficient for the production of a peroxidase (e.g., VHPO) results in one or more cells of the microorganism producing a peroxidase (e.g., VHPO).

Once the cell culture has achieved a desired level of growth, e.g., a specific growth phase or culture volume size, or when the cell culture, e.g., the aqueous portion, is substantially free from the carbon source, e.g., sugar, utilized to stimulate proliferation, the cell culture can be induced to produce a protein, e.g., a peroxidase (e.g., VHPO) described herein. A composition described herein comprising a small organic compound starting material is added e.g., fed, to the host cell microorganism or cell culture that is capable of producing a peroxidase (e.g., VHPO), thereby inducing the host cell microorganism to produce the peroxidase (e.g., VHPO). In an embodiment, the composition comprising a small organic compound is added to the culture directly. In an embodiment, the composition comprising a small organic compound is added to an enzyme production culture media, comprising components that support and encourage the production of the protein, e.g., peroxidase (e.g., VHPO). The host cell microorganism is then transferred or cultured in the enzyme production culture media. An enzyme production culture media can comprise physiological salts, e.g., ammonium salts, and a composition comprising a caramelized sugar product and/or a second agent, and is adjusted to pH 4-7, e.g., pH 6. In an embodiment, an enzyme production culture media comprises ammonium sulfate, rice bran, and a composition comprising a small organic compound.

In an embodiment, a small organic compound and a second agent are added to the host cell microorganism or cell culture simultaneously. The small organic compound and second agent (e.g., a peroxide-containing substrate, e.g., H₂O₂ or PAA) can be present in the same composition or can be in separate compositions. When the small organic compound and second agent are present in the same composition, the small organic compound and second agent can be components of an enzyme production culture media. In another embodiment, a small organic compound and a second agent are in separate compositions and are added to the host cell microorganism or cell culture sequentially. For example, a small organic compound can be added to the host cell microorganism or cell culture prior to or after a second agent is added to the host cell microorganism or cell culture. In such sequential induction processes, the duration between the addition of the small organic compound and the addition of a second agent can be hours, e.g., 1, 2, 3, 4, 5, 6, 12, 18, or more hours, or days, e.g., 1, 2, 3, 4, 5, 6, 7 or more days.

A small organic compound can be introduced to the host cell microorganism, e.g., by direct addition to the culture or by enzyme production culture media, twice a day, once a day, every other day, every three days or once a week. The small organic compound can be added at a concentration range of 1-20 g/L, 1-15 g/L, 1-10 g/L, 1-5 g/L, 2-15 g/L, 2-10 g/L, 2-5 g/L, 5-20 g/L, 5-15 g/L, 5-10 g/L, 4-5 g/L, 10-20 g/L or 10-15 g/L of host cell microorganism cell culture. The small organic compound can be added at a concentration of 0.5 g/L, 1 g/L, 2 g/L, 3 g/L, 4 g/L, 5 g/L, 6 g/L, 7 g/L, 8 g/L, 9 g/L, 10 g/L, 15 g/L or 20 g/L or more, of host cell microorganism culture. In an embodiment, the small organic compound is added to the host cell microorganism at 4 g/L once per day, or 5 g/L once per day.

A second agent (e.g., a peroxide-containing substrate, e.g., H₂O₂ or PAA) can be introduced to the host cell microorganism, e.g., by direct addition to the culture or by enzyme production culture media, twice a day, once a day, every other day, every 3 days, or once a week. The second agent can be added at a concentration range of 1-20 g/L, 1-15 g/L, 1-10 g/L, 1-5 g/L, 2-15 g/L, 2-10 g/L, 2-5 g/L, 5-20 g/L, 5-15 g/L, 5-10 g/L, 10-20 g/L, or 10-15 g/L of host cell microorganism cell culture. The second agent can be added at a concentration of 0.5 g/L, 1 g/L, 2 g/L, 3 g/L, 4 g/L, 5 g/L, 6 g/L, 7 g/L, 8 g/L, 9 g/L, 10 g/L, 15 g/L or 20 g/L or more, of host microorganism culture. In an embodiment, the second agent is added to the host cell microorganism at 5 g/L, once per day.

In embodiments, the concentration of a small organic compound or a second agent used for inducing production of a peroxidase (e.g., VHPO) is greater than or equal to 0.1% weight by volume (w/v), 0.5% w/v, 1% w/v, 2% w/v, or 5% w/v, and less than or equal to 25% w/v, 20% w/v, 15% w/v, and 10% w/v. The host cell microorganism can be induced to produce a peroxidase (e.g., VHPO) for one or more days, e.g., 2, 3, 4, 5, 6, 7, 8, 9, 10, 11, 12, 13, 14, 15, 20, or 30 or more days. The duration of the induction can depend on the size, e.g., volume or cell number, of the host cell microorganism culture, the microorganism used, or the amount of the peroxidase (e.g., VHPO) needed. In an embodiment, the host cell microorganism is induced to produce a peroxidase (e.g., VHPO) for 1-2, 1-3, 1-4, 1-5, 1-6, 1-7, 1-8, 1-9, 1-10, 1-11 or 1-12 days.

Production of the peroxidase (e.g., VHPO) can be measured from the cell culture by measuring the level of peroxidase (e.g., VHPO) present in the cell culture and/or activity of the peroxidase (e.g., VHPO) that is produced by the cells. For example, the aqueous portion of the culture can be isolated, e.g., by centrifuging the cell culture or an aliquot or sample of the cell culture. A protein assay known in the art, such as the Bradford assay or nanodrop protein quantification, can be used to determine the total level or titer of protein, e.g., g/L, in the aqueous portion of the culture. The total protein titer indicates the amount of peroxidase (e.g., VHPO) produced by the microorganism or cell culture. A control sample can be used to normalize for the amount of proteins present in a cell culture that has not been induced to produce a peroxidase (e.g., VHPO).

The peroxidases (e.g., VHPOs) produced by the host cell microorganism as described herein can be useful in biological or industrial processes, e.g. the peroxidase enzyme itself or the products of peroxidase reactions may be used in a rumen composition as described herein. Methods for modulating features of the rumen, including reducing the production of methane in the rumen, are described further herein. The host cell microorganism, or culture thereof, that has been induced to produce a peroxidase (e.g., VHPO), as described herein, can be added directly to the rumen. Alternatively, the small organic compound or a plurality of small organic compounds to be processed can be added directly to the host cell microorganism or culture that has been induced to produce a peroxidase (e.g., VHPO). In another aspect the isolated halogenated organic compound produced as a product of peroxidase catalysis is added to the rumen as inhibitor of methanogenesis. In another aspect the halogenated organic compound produced as a product of peroxidase catalysis is delivered to the rumen via encapsulation in a matrix for inhibition of methanogenesis.

A peroxidase (e.g., VHPO) produced by the host cell microorganism as described herein can also be separated or purified prior use in a culture or rumen sample. The peroxidase (e.g., VHPO) can be separated from one or more of the following components: the microorganism, e.g., the cells of the microorganism; the small organic compound starting material; the small organic halogenated compound product; the second agent; components of the cell culture media, e.g., glucose, physiological salts; and one or more proteins present in the culture that do not have peroxidase activity. The peroxidase (e.g., VHPO) can be purified, such that the peroxidase (e.g., VHPO) is substantially free of other proteins that do not have peroxidase activity, cell debris, nucleic acids, e.g., from the host cell microorganism, small organic compound, and/or agent. Methods for separation or purification of a peroxidase (e.g., VHPO) are known in the art, and can include centrifugation, filtration, protein fractionation, size exclusion chromatography, affinity chromatography, ion exchange chromatography, or any combination thereof.

### Halogenation of Compounds

The present disclosure features methods for producing small halogenated organic compounds, as well as related compositions thereof, using a peroxidase. In an embodiment, the method comprises (i) providing a small organic compound or a plurality of small organic compounds; (ii) providing a peroxide source, e.g. H₂O₂ or peracetic acid (PAA), and a halogen source, e.g. halogenated salts such as KBr, NaCl or KI; (iii) contacting the small organic compound or plurality of small organic compounds with a peroxidase, such as a vanadium haloperoxidase (i.e., VHPO), or the reaction product thereof, i.e. hypohalous acid to form a reaction mixture under conditions sufficient to produce a small halogenated organic compound or a plurality of small halogenated organic compounds; and (iv) evaluating the small halogenated organic compound or plurality of small halogenated organic compounds produced.

The small organic compound may be a naturally occurring or non-naturally occurring compound. For example, the small organic compound may comprise be a natural product, a lipid, a sterol, a steroid, an amino acid, a sugar, a phlorotannin, a tannin, a lignin, or a lignin derivative. In an embodiment, the small organic compound comprises a functional group, e.g., an aldehyde, ketone, acetyl, acyl, hydroxyl, ester, ether, amine, amide, aryl, heteroaryl, heterocyclyl, or cycloalkyl group. In an embodiment, the small organic compound comprises an alkenyl or alkynyl group. In an embodiment, the small organic compound comprises an aldehyde or ketone group. In an embodiment, the small organic compound comprises an alpha-beta unsaturated ketone. In an embodiment, the small organic compound is acetone or acetylacetone.

The small organic compound contains at least 1 carbon atom, e.g., at least 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 12 carbon atoms, or more. In an embodiment, the small organic compound comprises between 1 and 10 carbon atoms, 1 and 6 carbon atoms, 2 and 10 carbon atoms, 3 and 10 carbon atoms, 4 and 10 carbon atoms, 5 and 10 carbon atoms, or 6 and 10 carbon atoms. In some embodiments, the small organic compound is saturated. In In some embodiments, the small organic compound comprises an element of unsaturation, e.g., 2, 3, 4, 5, 6, 7, 8, or more elements of unsaturation. In an embodiment, the small organic compound further comprises an oxygen atom, a nitrogen atom, a sulfur atom, or a phosphorus atom. In an embodiment, the small organic compound comprises 1, 2, 3, 4, 5, or 6 oxygen atoms. In an embodiment, the small organic compound comprises 1, 2, 3, 4, 5, or 6 nitrogen atoms. In an embodiment, the small organic compound comprises 1, 2, 3, 4, 5, or 6 sulfur atoms. In an embodiment, the small organic compound comprises 1, 2, 3, 4, 5, or 6 phosphorus atoms.

In an embodiment, the small organic compound has a molecular weight or molecular mass of between about 15 Da and 1,500 Da, e.g., between about 15 Da and 1,250 Da, between about 15 Da and about 1,000 Da, between about 15 Da and about 750 Da, between about 15 Da and about 500 Da, between about 15 Da and about 250 Da, between about 15 Da and about 100 Da, between about 25 Da and 500 Da, between about 25 Da and 100 Da, between about 50 Da and 500 Da, between about 100 Da and 250 Da, between about 100 Da and 500 Da, or between about 100 Da and 1,000 Da.

In an embodiment, the small organic compound comprises an aldehyde, ketone, acetyl, acyl, hydroxyl, ester, ether, amine, amide, aryl, heteroaryl, heterocyclyl, or cycloalkyl group. In an embodiment, the small organic compound comprises an alkenyl or alkynyl group. In an embodiment, the small organic compound comprises an aldehyde or ketone group. In an embodiment, the small organic compound comprises an alpha-beta unsaturated ketone. In an embodiment, the small organic compound is acetone or acetylacetone. In an embodiment, the small organic compound is a natural product, a lipid, a sterol, a steroid, an amino acid, a sugar, a phlorotannin, a tannin, a lignin, or a lignin derivative.

In one aspect, the small organic compound comprises a compound of Formula (Y):

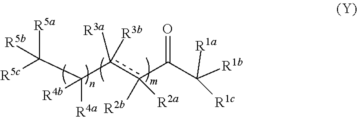

or a salt, tautomer, or isomer thereof, wherein each of R^{1a}, R^{1b}, R^{1c}, R^{2a}, R^{2b}, R^{3a}, R^{3b}, R^{4a}, R^{4b}, R^{5a}, R^{5b}, and R^{5c} is independently hydrogen, halogen, C₁-C₆ alkyl, C₁-C₆ heteroalkyl, C₂-C₆ alkenyl, C₂-C₆ alkynyl, cycloalkyl, or heterocyclyl, wherein each alkyl, heteroalkyl, alkenyl, alkynyl, cycloalkyl or heterocyclyl is optionally substituted with one or more R⁶; R⁶ is halogen, C₁-C₆ alkyl, C₁-C₆ heteroalkyl, C₂-C₆ alkenyl, C₂-C₆ alkynyl, —OR^{A}, or —NR^{B}R^{C}; R^{A} is hydrogen, C₁-C₆ alkyl, C₁-C₆ heteroalkyl, or C₂-C₆ alkenyl; R^{B} and R^{C} are each independently hydrogen, C₁-C₆ alkyl, or C₁-C₆ heteroalkyl; each of m and n is independently an integer between 0 and 24; and “ [symbol] ” is a single or double bond, wherein when [symbol] is a double bond, each of R^{2b} and R^{3b} is independently absent.

In an embodiment of Formula (Y), each of R^{1a}, R^{1b}, R^{1c}, R^{2a}, R^{2b}, R^{3a}, R^{3b}, R^{5a}, R^{5b}, and R^{5c} are each independently hydrogen. In an embodiment of Formula (Y), m is selected from 0, 1, 2, or 3. In an embodiment of Formula (Y), n is selected from 0, 1, 2, or 3. In an embodiment of Formula (Y), [symbol] is a single bond. In an embodiment of Formula (Y), each of R^{1a}, R^{1b}, R^{1c}, R^{2a}, R^{2b}, R^{3a}, R^{3b}, R^{5a}, R^{5b}, and R⁵⁰ are each independently hydrogen, each of m and n is independently selected from 0, 1, 2, or 3, and [symbol] is a single bond.

In an embodiment of Formula (Y), each of R^{1a}, R^{1b}, R^{1c}, R^{5a}, R^{5b}, and R⁵⁰ are each independently hydrogen, and each of m and n is 0. In an embodiment of Formula (Y), each of R^{1a}, R^{1b}, R^{1c}, R^{2a}, R^{2b}, R^{3a}, R^{3b}, R^{5a}, R^{5b}, and R⁵⁰ are each independently hydrogen, n is 0, m is 1, and [symbol] is a single bond. In an embodiment of Formula (Y), R^{1a} is C₁ alkyl; R^{1b}, R^{1c}, R^{5a}, R^{5b}, and R⁵⁰ are each independently hydrogen, and each of m and n is 0. In an embodiment of Formula (Y), R^{1a} is halogen (e.g., chlorine, bromine, or iodine); R^{1b}, R^{1c}, R^{5a}, R^{5b}, and R^{5c} are each independently hydrogen, and each of m and n is 0.

In an embodiment, the method features providing a peroxide source, e.g. PAA, to a VHPO (e.g., a VCPO), e.g., a VHPO comprising a protein sequence motif provided in Table 1 or a VHPO sequence provided in Table 2. In an embodiment, the method features: (i) providing a peroxide source, e.g. PAA, and a chloride anion to a VCPO to generate a hypochlorite anion or its conjugate acid. In an embodiment, the method features: (i) providing a peroxide source, e.g. PAA, and a chlorine source to a VCPO to generate an hypochlorite anion or its conjugate acid; and (ii): providing the hypochlorite anion to an amine to generate a chlorinated amine, e.g. NH₂Cl. In an embodiment, the method features: (i) providing a peroxide source, e.g. PAA, and a chlorine source to a VCPO to generate an hypochlorite anion or its conjugate acid; (ii): providing the hypochlorite anion to an amine to generate a chlorinated amine, e.g. NH₂Cl; and (iii) reacting the chlorinated amine with a iodide anion to generate a hypoiodite anion or its conjugate acid. In an embodiment, the method features: (i) providing a peroxide source, e.g. PAA, and a chlorine source to a VCPO to generate an hypochlorite anion or its conjugate acid; and (ii-a) reacting the hypochlorite anion with an iodide anion to generate a hypoiodite anion or its conjugate acid. In an embodiment, the method features: (i) providing a peroxide source, e.g. PAA, and a chlorine source to a VCPO to generate an hypochlorite anion or its conjugate acid; (ii): providing the hypochlorite anion to an amine to generate a chlorinated amine, e.g. NH₂Cl; (iii) reacting the chlorinated amine with a iodide anion to generate a hypoiodite anion or its conjugate acid; and (iv) reacting the hypoiodite anion with the small organic compound dichloroacetic acid to generate the small halogenated organic compound product dichloroiodomethane. In an embodiment, the method features: (i) providing a peroxide source, e.g. PAA, and a chlorine source to a VCPO to generate an hypochlorite anion or its conjugate acid; (ii-a) reacting the hypochlorite anion with an iodide anion to generate a hypoiodite anion or its conjugate acid; and (iv)) reacting the hypoiodite anion with the small organic compound dichloroacetic acid to generate the small halogenated organic compound product dichloroiodomethane. In an embodiment, the method features (ii). In an embodiment, the method features (ii) and (iii). In an embodiment, the method features (ii), (iii), and (iv). In an embodiment, the method features (ii) and (iv). In an embodiment, the method features (i) and (iii). In an embodiment, the method features (i), (iii), and (iv). In an embodiment, the method features (iii) and (iv). In an embodiment, the method features (ii-a). In an embodiment, the method features (ii-a) and (iv). In an embodiment, the method features (iii). In an embodiment, the method features (iv).

In an embodiment the method comprising the enzymatic reaction of PAA and VCPO features a reduction between 10%-99% in the conversion rate of hypohalite anion and excess iodide anion to diatomic iodine I₂ and triiodide anion I₃⁻ at pH between 0-5 over the method comprising the enzymatic reaction of H₂O₂ and VHPO at pH between 0-5. In an embodiment the method comprising the enzymatic reaction of PAA and VCPO features a reduction between 10%-99% in the conversion rate of hypohalite anion and excess iodide anion to diatomic iodine I₂ and triiodide anion I₃⁻ at pH 7 over the method comprising the enzymatic reaction of H₂O₂ and VHPO at pH 7. In an embodiment the method comprising the enzymatic reaction of PAA and VCPO features a reduction between 10%-99% in the conversion rate of hypohalite anion and bromide anion to diatomic bromine Br₂ at pH between 0-5 over the method comprising the enzymatic reaction of H₂O₂ and VHPO at pH between 0-5. In an embodiment the method comprising the enzymatic reaction of PAA and VCPO features a reduction between 10%-99% in the conversion rate of hypohalite anion and chloride anion to diatomic chlorine Cl₂ at pH between 0-5 over the method comprising the enzymatic reaction of H₂O₂ and VHPO at pH between 0-5. In an embodiment the method comprising the enzymatic reaction of PAA and VCPO features a reduction between 10%-99% in the conversion rate of peracetic acid and bromide anion to hypobromite anion or its conjugate acid at pH between 0-5 over the method comprising the enzymatic reaction of H₂O₂ and VHPO at pH between 0-5. In an embodiment the method comprising the enzymatic reaction of PAA and VCPO features a reduction between 10%-99% in the conversion rate of peracetic acid and iodide anion to hypoiodite anion or its conjugate acid at pH between 0-5 over the method comprising the enzymatic reaction of PAA and VHPO at pH between 0-5.

In an embodiment the method comprising the enzymatic reaction of PAA and VCPO features a reduction between 10%-99% in the conversion rate of hypochlorite and hypoiodite to iodite anion IO₂⁻ and iodate anion IO₃⁻ at pH between 7-14 over the method comprising the enzymatic reaction of H₂O₂ and VHPO at pH between 7-14. In an embodiment the method comprising the enzymatic reaction of PAA and VCPO features a reduction between 10%-99% in the conversion rate of hypobromite and hypoiodite to iodite anion IO₂⁻ and iodate anion IO₃⁻ at pH between 7-14 over the method comprising the enzymatic reaction of H₂O₂ and VHPO at pH between 7-14. In an embodiment the method comprising the enzymatic reaction of PAA and VCPO features a reduction between 10%-99% in the conversion rate of hypoiodite and hypoiodite to iodite anion IO₂⁻ and iodate anion IO₃⁻ at pH between 7-14 over the method comprising the enzymatic reaction of H₂O₂ and VHPO at pH between 7-14. In an embodiment the method comprising the enzymatic reaction of PAA and VCPO features a reduction between 10%-99% in the conversion rate of hypohalite and hypoiodite to iodite anion IO₂⁻ and iodate anion IO₃⁻ at pH between 7-14 over the method comprising the enzymatic reaction of H₂O₂ and VHPO at pH between 7-14. In an embodiment the method comprising the enzymatic reaction of PAA and VCPO features a reduction between 10%-99% in the conversion rate of a hypohalite comprising HOCl, HOBr, or HOI, or a combination thereof, and hypoiodite to iodite anion IO₂⁻ and iodate anion IO₃⁻ at pH between 7-14 over the method comprising the enzymatic reaction of H₂O₂ and VHPO at pH between 7-14. In an embodiment the method comprising the enzymatic reaction of PAA and VCPO features a reduction between 10%-99% in the conversion rate of monochloroamine and hypoiodite to iodite anion IO₂⁻ and iodate anion IO₃⁻ at pH between 7-14 over the method comprising the enzymatic reaction of H₂O₂ and VHPO at pH between 7-14. In an embodiment the method comprising the enzymatic reaction of PAA and VCPO features a reduction between 10%-99% in the conversion rate of diatomic iodine I₂ and hypoiodite to iodite anion IO₂⁻ and iodate anion IO₃⁻ at pH between 7-14 over the method comprising the enzymatic reaction of H₂O₂ and VHPO at pH between 7-14.

In an embodiment the method comprising the enzymatic reaction of PAA and VCPO features an increase between 10%-500% in the k_{cat} over the method comprising the enzymatic reaction of H₂O₂ and VHPO. In an embodiment the method comprising the enzymatic reaction of PAA and VCPO features an increase between 25%-250% in the k_{cat} over the method comprising the enzymatic reaction of H₂O₂ and VHPO. In an embodiment the method comprising the enzymatic reaction of PAA and VCPO features an increase between 50%-150% in the k_{cat} over the method comprising the enzymatic reaction of H₂O₂ and VHPO. In an embodiment the method comprising the enzymatic reaction of PAA and VCPO features an increase between 75%-125% in the k_{cat} over the method comprising the enzymatic reaction of H₂O₂ and VHPO. In an embodiment the method comprising the enzymatic reaction of PAA and VCPO features an increase between 80%-120% in the k_{cat} over the method comprising the enzymatic reaction of H₂O₂ and VHPO. In an embodiment the method comprising the enzymatic reaction of PAA and VCPO features an increase between 90%-110% in the k_{cat} over the method comprising the enzymatic reaction of H₂O₂ and VHPO.

In an embodiment the method comprising the enzymatic reaction of PAA and VCPO features an increase between 10%-500% in the k_{cat} over the method comprising the enzymatic reaction of H₂O₂ and VHPO, wherein dichloriodomethane is generated. In an embodiment the method comprising the enzymatic reaction of PAA and VCPO features an increase between 25%-250% in the k_{cat} over the method comprising the enzymatic reaction of H₂O₂ and VHPO, wherein dichloriodomethane is generated. In an embodiment the method comprising the enzymatic reaction of PAA and VCPO features an increase between 50%-150% in the k_{cat} over the method comprising the enzymatic reaction of H₂O₂ and VHPO, wherein dichloriodomethane is generated. In an embodiment the method comprising the enzymatic reaction of PAA and VCPO features an increase between 75%-125% in the k_{cat} over the method comprising the enzymatic reaction of H₂O₂ and VHPO, wherein dichloriodomethane is generated. In an embodiment the method comprising the enzymatic reaction of PAA and VCPO features an increase between 80%-120% in the k_{cat} over the method comprising the enzymatic reaction of H₂O₂ and VHPO, wherein dichloriodomethane is generated. In an embodiment the method comprising the enzymatic reaction of PAA and VCPO features an increase between 90%-110% in the k_{cat} over the method comprising the enzymatic reaction of H₂O₂ and VHPO, wherein dichloriodomethane is generated. In an embodiment the method comprising the enzymatic reaction of PAA and VCPO features an increase between 10%-500% in the k_{cat} over the method comprising the enzymatic reaction of H₂O₂ and VHPO, wherein dibromochloromethane is generated. In an embodiment the method comprising the enzymatic reaction of PAA and VCPO features an increase between 25%-250% in the k_{cat} over the method comprising the enzymatic reaction of H₂O₂ and VHPO, wherein dibromochloromethane is generated. In an embodiment the method comprising the enzymatic reaction of PAA and VCPO features an increase between 50%-150% in the k_{cat} over the method comprising the enzymatic reaction of H₂O₂ and VHPO, wherein dibromochloromethane is generated. In an embodiment the method comprising the enzymatic reaction of PAA and VCPO features an increase between 75%-125% in the k_{cat} over the method comprising the enzymatic reaction of H₂O₂ and VHPO, wherein dibromochloromethane is generated. In an embodiment the method comprising the enzymatic reaction of PAA and VCPO features an increase between 80%-120% in the k_{cat} over the method comprising the enzymatic reaction of H₂O₂ and VHPO, wherein dibromochloromethane is generated. In an embodiment the method comprising the enzymatic reaction of PAA and VCPO features an increase between 90%-110% in the k_{cat} over the method comprising the enzymatic reaction of H₂O₂ and VHPO, wherein dibromochloromethane is generated.

As described herein, the peroxidase (e.g., VHPO) may be used to convert a small organic compound into a small halogenated organic compound, e.g., in the presence of a halogen source. In an embodiment, the small halogenated organic compound comprises a natural product, a lipid, a sterol, a steroid, an amino acid, a sugar, a phlorotannin, a tannin, a lignin, or a lignin derivative is chlorinated, brominated, or iodinated. In an embodiment, the small halogenated organic compound is brominated. In an embodiment, the small halogenated organic compound comprises a functional group, e.g., an aldehyde, ketone, acetyl, acyl, hydroxyl, ester, ether, amine, amide, aryl, heteroaryl, heterocyclyl, or cycloalkyl group. In an embodiment, the small halogenated organic compound comprises an alkenyl or alkynyl group. In an embodiment, the small halogenated organic compound comprises an aldehyde or ketone group. In an embodiment, the small halogenated organic compound comprises an alpha-beta unsaturated ketone. In an embodiment, the small halogenated organic compound comprises 1, 2, 3, 4, 5, 6, 7, or 8 carbon atoms. In an embodiment, the small halogenated organic compound comprises 1, 2, 3 halogen atoms. In an embodiment, the small halogenated organic compound comprises 1, 2, 3 bromine atoms. In an embodiment, the small halogenated organic compound comprises an acetone moiety. In an embodiment, the small halogenated organic compound comprises dibromoacetone, bromoacetone, bromopentanedione, bromoform, or tribromoacetone. In an embodiment, the small halogenated organic compound comprises 1,1-dibromoacetone, bromoacetone, 3-bromo-2,4-pentanedione, bromoform, 1,1,3-tribromoacetone, or 1,1,1-tribromoacetone. In an embodiment, the small halogenated compound comprises dichloroiodomethane, dichlorobromomethane, dibromoiodomethane, diiodochloromethane, or diiodobromomethane. In an embodiment, the small halogenated organic compound comprises 1,1-dibromoacetone, bromoacetone, 3-bromo-2,4-pentanedione, bromoform, 1,1,3-tribromoacetone, or 1,1,1-tribromoacetone, dichloroiodomethane, dichlorobromomethane, dibromoiodomethane, diiodochloromethane, or diiodobromomethane.

The small halogenated organic compound may comprise any halogen atom, such as a chlorine atom, a bromine atom, or an iodine atom. In an embodiment, the small halogenated organic compound comprises 1, 2, 3, 4, 5, 6, or more halogen atoms. In an embodiment, the small halogenated organic compound comprises 1, 2, 3, 4, 5, 6, or more chlorine atoms. In an embodiment, the small halogenated organic compound comprises 1, 2, 3, 4, 5, 6, or more bromine atoms. In an embodiment, the small halogenated organic compound comprises 1, 2, 3, 4, 5, 6, or more iodine atoms. In an embodiment, the small halogenated organic compound comprises bromoacetone, dibromoacetone, bromopetandione, bromoform, tribromoacetone, or a variant or analog thereof. In an embodiment, the small halogenated organic compound comprises 1,1-dibromoacetone, bromoacetone, 3-bromo-2,4-pentanedione, bromoform, 1,1,3-tribromoacetone, 1,1,1-tribromoacetone, or a variant or analog thereof. In an embodiment, the small halogenated organic compound comprises halomethanes, e.g. dihalomethanes and trihalomethanes. In an embodiment, the small halogenated organic compound comprises haloacetones, e.g. dihaloacetones and trihaloacetones. In an embodiment, the small halogenated organic compound comprises bromoacetone, 1,1-dibromoacetone, 1,1,3-tribromoacetone, 1,1,1-tribromoacetone, or a variant or analog thereof. In an embodiment, the small halogenated organic compound comprises bromomethane, dibromethane, bromoform, or a variant or analog thereof.

In another aspect, the small halogenated organic compound comprises a compound of Formula (Z):

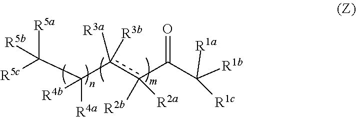

or a salt, tautomer, or isomer thereof, wherein each of R^{1a}, R^{1b}, R^{1c}, R^{2a}, R^{2b}, R^{3a}, R^{3b}, R^{4a}, R^{4b}, R^{5a}, R^{5b}, and R^{5c} is independently hydrogen, halogen, C₁-C₆ alkyl, C₁-C₆ heteroalkyl, C₂-C₆ alkenyl, C₂-C₆ alkynyl, cycloalkyl, or heterocyclyl, wherein each alkyl, heteroalkyl, alkenyl, alkynyl, cycloalkyl or heterocyclyl is optionally substituted with one or more R⁶, and at least one of R^{1a}, R^{1b}, R^{1c}, R^{2a}, R^{2b}, R^{3a}, R^{3b}, R^{4a}, R^{4b}, R^{5a}, R^{5b}, and R^{5c} is independently halogen; R⁶ is halogen, C₁-C₆ alkyl, C₁-C₆ heteroalkyl, C₂-C₆ alkenyl, C₂-C₆ alkynyl, —OR^{A}, or —NR^{B}R^{C}; R^{A} is hydrogen, C₁-C₆ alkyl, C₁-C₆ heteroalkyl, or C₂-C₆ alkenyl; R^{B} and R^{C} are each independently hydrogen, C₁-C₆ alkyl, or C₁-C₆ heteroalkyl; each of m and n is independently selected from 0, 1, 2, or 3; and “ [symbol] ” is a single or double bond, wherein when [symbol] is a double bond, each of R^{2b} and R^{3b} is independently absent.

In an embodiment of Formula (Z), each of R^{1a}, R^{1b}, and R^{1c} is independently halogen or hydrogen, wherein at least one of R^{1a}, R^{1b}, and R^{1c} is halogen. In an embodiment, the halogen is selected from chlorine, bromine, or iodine. In an embodiment of Formula (Z), each of R^{1a}, R^{1b}, R^{1c} is independently halogen or hydrogen, wherein at least two of R^{1a}, R^{1b}, and R^{1c} is halogen. In an embodiment, the halogen is selected from two of chlorine, bromine, or iodine. In an embodiment of Formula (Z), each of R^{1a}, R^{1b}, R^{1c} is independently halogen. In an embodiment, the halogen is selected from chlorine, bromine, or iodine. In an embodiment of Formula (Z), each of R^{5a}, R^{5b}, and R^{5c} is independently halogen or hydrogen, wherein at least one of R^{5a}, R^{5b}, and R^{5c} is halogen. In an embodiment of Formula (Z), each of R^{5a}, R^{5b}, and R^{5c} is independently halogen or hydrogen, wherein at least two of R^{5a}, R^{5b}, and R^{5c} is halogen. In an embodiment of Formula (Z), each of R^{5a}, R^{5b}, and R^{5c} is independently halogen. In an embodiment of Formula (Z), [symbol] is a single bond. In an embodiment of Formula (Z), each of m and n is independently selected from 0, 1, 2, or 3, and [symbol] is a single bond.

The peroxidases (e.g., VHPOs) described herein are capable of modulating the production of a small halogenated organic compound based on the reaction conditions. For example, under a first set of reaction conditions, the peroxidase may produce a first small halogenated organic compound, and under a second set of reaction conditions, the peroxidase may produce a second halogenated organic compound. In addition, the peroxidases (e.g., VHPOs) described herein may be capable of modulating the ratio of certain small halogenated organic compounds to one another within a plurality of small halogenated organic compounds, depending on the reaction conditions. For example, under a first set of reaction conditions, the peroxidase may produce a first ratio of a small halogenated organic compound to another small halogenated organic compound within the plurality, and under a second set of reaction conditions, the peroxidase may produce a second ratio of a small halogenated organic compound to another small halogenated organic compound within the plurality.

In an embodiment, the peroxidase (e.g., VHPO) capable of modulating the production of a small halogenated organic compound is a peroxidase derived from an algal species. In an embodiment, the peroxidase (e.g., VHPO) capable of modulating the production of a small halogenated organic compound is a peroxidase derived from a fungal species. In an embodiment, the peroxidase (e.g., VHPO) capable of modulating the production of a small halogenated organic compound is a peroxidase derived from a cyanobacterium species In an embodiment, the peroxidase (e.g., VHPO) capable of modulating the production of a small halogenated organic compound is from C. inaequalis, C. officinalis, N. carneum, H. hongdechloris, M. bouillonii, T. erythraeum, A. montana, Synechococcus, S. cerevisiae, L. confervoides, or Lyngbya. In an embodiment, the peroxidase (e.g., the VHPO) capable of modulating the production of a small halogenated organic compound comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to an amino acid sequence listed in Table 1, e.g., provided herein. In an embodiment, the peroxidase (e.g., VHPO) capable of modulating the production of a small halogenated organic compound is produced in a host cell microorganism described herein, e.g., P. pastoris, A. niger, or S. cerevisiae.

The halogenation of a small organic compound or a plurality of small organic compounds takes place under certain conditions, e.g., conditions sufficient for halogenation. In an embodiment, the conditions sufficient for producing a small halogenated organic compound or a plurality of small halogenated organic compounds comprise a temperature between 0° C. and 85° C., e.g., between about 4° C. and about 65° C., between about 10° C. and about 65° C., and between about 25° C. and about 50° C. In an embodiment, the conditions sufficient for producing a small halogenated organic compound or a plurality of small halogenated organic compounds comprise a pH between about 2 and about 10, e.g., between about 2 and about 9, between about 2 and about 8, between about 4 and about 10, between about 4 and about 8, between about 5 and about 8.

### ENUMERATED EMBODIMENTS

1. A method of modulating production of a small halogenated organic compound with a peroxidase enzyme, comprising:
(i) providing a small organic compound (e.g., acetyl acetone);
(ii) contacting the small organic compound with a peroxidase (e.g., a VHPO) to form a reaction mixture under conditions sufficient to produce a small halogenated organic compound;
(iii) evaluating the small halogenated organic compound produced; and thereby modulating production of a small halogenated organic compound.
   2. The method of embodiment 1, wherein the peroxidase is a haloperoxidase.
   3. The method of embodiment 2, wherein the haloperoxidase is a vanadium haloperoxidase (VHPO).
   4. The method of embodiment 3, wherein the VHPO is a vanadium chloroperoxidase (VCPO), vanadium bromoperoxidase (VBPO), or vanadium iodoperoxidase (VHPO).
   5. The method of any one of embodiments 3-4, wherein the VHPO is a VBPO.
   6. The method of any one of the preceding embodiments, wherein the peroxidase is an algal haloperoxidase (e.g., derived from an algal species), a fungal haloperoxidase (e.g., derived from a fungal species), or a cyanobacterial haloperoxidase (e.g. derived from a cyanobacteria).
   7. The method of any one of the embodiments, wherein the peroxidase is a fungal haloperoxidase (e.g., derived from a fungal species).
   8. The method of any one of the preceding embodiments, wherein the peroxidase is derived from an organism selected from Curvularia inaequalis, Halomicronema hongdechloris, Moorea bouillonii, Trichodesmium erythraeum, Aphanocapsa montana, Lyngbya confervoides, Synechococcus sp. PCC7335, and Corallina officinalis.
   9. The method of any one of the preceding embodiments, wherein the peroxidase is derived from Corallina officinalis.
   10. The method of any one of the preceding embodiments, wherein the peroxidase is derived from Aphanocapsa montana.
   11. The method of any one of the preceding embodiments, wherein the peroxidase is derived from Curvularia inaequalis.
   12. The method of any one of the preceding embodiments, wherein the peroxidase is produced in a host cell microorganism.
   13. The method of any one of the preceding embodiments, wherein the host cell microorganism is selected from Pichia pastoris, Aspergillus niger, Saccharomyces cerevisiae, or Escherichia coli.
   14. The method of any one of the preceding embodiments, wherein expression of the peroxidase produced in the host cell microorganism is increased by about 1.5-fold, 2-fold, 3-fold, 4-fold, 5-fold, 6-fold, 7-fold, 8-fold, or 10-fold, e.g., over a peroxidase produced in its native host.
   15. The method of any one of the preceding embodiments, wherein the amino acid sequence of the peroxidase comprises a motif selected from the protein sequence motifs provided in Table 1.
   16. The method of any one of the preceding embodiments, wherein the amino acid sequence of the peroxidase is selected from an amino acid sequence listed in Table 2.
   17. The method of any one of the preceding embodiments, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from the list in Table 2.
   18. The method of any one of the preceding embodiments, wherein the peroxidase is a sequence selected from any one of SEQ ID NOs. 1-50.
   19. The method of any one of the preceding embodiments, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from SEQ ID NOs: 1-50.
   20. The method of any one of the preceding embodiments, wherein the amino acid sequence of the peroxidase has at 1, 2, 3, 4, 5, or 6 amino acid substitutions relative to an amino acid sequence selected from any one of SEQ ID NOs. 1-50.
   21. The method of any one of the preceding embodiments, wherein the small organic compound comprises 1, 2, 3, 4, 5, 6, 7, or 8 carbon atoms.
   22. The method of any one of the preceding embodiments, wherein the small organic compound comprises a ketone or aldehyde.
   23. The method of any one of the preceding embodiments, wherein the small halogenated organic compound is chlorinated; brominated; iodinated; chlorinated and iodinated; chlorinated and brominated; brominated and iodinated; or chlorinated, brominated, and iodinated.
   24. The method of any one of the preceding embodiments, wherein the small halogenated organic compound is brominated.
   25. The method of any one of the preceding embodiments, wherein the small halogenated organic compound comprises 1, 2, 3 halogen atoms.
   26. The method of any one of the preceding embodiments, wherein the small halogenated organic compound comprises 1, 2, 3 bromine atoms.
   27. The method of any one of the preceding embodiments, wherein the small halogenated organic compound comprises an acetone moiety.
   28. The method of any one of the preceding embodiments, wherein the small halogenated organic compound comprises dibromoacetone, bromoacetone, bromopentanedione, bromoform, or tribromoacetone.
   29. The method of any one of the preceding embodiments, wherein the small halogenated organic compound comprises dichloroiodomethane, dibromochloromethane, 1,1-dibromoacetone, bromoacetone, 3-bromo-2,4-pentanedione, bromoform, 1,1,3-tribromoacetone, or 1,1,1-tribromoacetone.
   30. The method of any one of the preceding embodiments, further comprising providing a peroxide source, e.g. H₂O₂ or PAA, and a halogen source, e.g. halogenated salts such as KBr, NaCl or KI.
   31. The method of any one of the preceding embodiments, wherein the conditions sufficient to product a small halogenated organic compound comprise one or more of:
(a) temperature between 10° C. to 85° C.
(b) pH between 4-10; and
(c) an ionic strength between 0.1 mM to 4 M.
   32. The method of embodiment 31, wherein the conditions sufficient to product a small 25 halogenated organic compound comprise a temperature between 10° C. to 85° C.
   33. The method of any one of embodiments 31-32, wherein the conditions sufficient to product a small halogenated organic compound comprise a pH between 4-10.
   34. The method of any one of embodiments 31-33, wherein the conditions sufficient to product a small halogenated organic compound comprise an ionic strength between 0.1 mM to 4 M.
   35. The method of any one of the preceding embodiments, wherein the evaluating comprises analysis of the small halogenated organic compound by an analytical technique.
   36. The method of embodiment 45, wherein the analytical technique comprises HPLC, GC-MS, NMR.
   37. The method of any one of the preceding embodiments, wherein the small halogenated organic compound is capable of reducing methane production by a microbial organism.
   38. The method of embodiment 36, wherein methane production is reduced by at least 5%, 10% 15%, 20%, 25% 30%, 35%, 40%, 50%, 55%, 60%, 65%, 70%, 75%, 80%, 85%, 90%, 95% 99%, or more.
   39. The method of any one of embodiments 37-38, wherein the methane production is reduced by between 10-75%.
   40. The method of any one of embodiments 37-39, wherein the microbial organism is present within a rumen community.
   41. A method for reducing production of methane in a rumen community, comprising:
(i) providing a small organic compound (e.g., acetyl acetone);
(ii) contacting the small organic compound with a peroxidase (e.g., a VHPO) to form a reaction mixture under conditions sufficient to produce a small halogenated organic compound;
(iii) separating the small halogenated organic compound from the reaction mixture;
(iv) providing the small halogenated organic compound to a rumen community under conditions sufficient to reduce the production of methane.
   42. The method of embodiment 41, further comprising acquiring a value for the level of methane (a) prior to providing the peroxidase or (b) after providing the peroxidase.
   43. The method of embodiment 42, comprising (a).
   44. The method of embodiment 42, comprising (b).
   45. The method of embodiment 42, comprising (a) and (b).
   46. The method of any one of embodiments 41-45, wherein methane production is reduced by at least 5%, 10% 0.15%, 20%, 25% 30%, 35%, 40%, 50%, 55%, 60%, 65%, 70%, 75%, 80%, 85%, 90%, 95%, 99%, or more.
   47. The method of any one of embodiments 41-46, wherein the methane production is reduced by between 10-75%.
   48. The method of any one of embodiments 40-46, wherein the peroxidase is a haloperoxidase.
   49. The method of embodiment 47, wherein the haloperoxidase is a vanadium haloperoxidase (VHPO).
   50. The method of embodiment 49, wherein the VHPO is a vanadium chloroperoxidase (VCPO), vanadium bromoperoxidase (VBPO), or vanadium iodoperoxidase (VHPO).
   51. The method of any one of embodiments 49-50, wherein the VHPO is a VBPO.
   52. The method of any one of the preceding embodiments, wherein the peroxidase is an algal haloperoxidase (e.g., derived from an algal species) or a fungal haloperoxidase (e.g., derived from a fungal species).
   53. The method of any one of the preceding embodiments, wherein the peroxidase is a fungal haloperoxidase (e.g., derived from a fungal species).
   54. The method of any one of the preceding embodiments, wherein the peroxidase is derived from an organism selected from Curvularia inaequalis, Halomicronema hongdechloris, Moorea bouillonii, Trichodesmium erythraeum, Aphanocapsa montana, Lyngbya confervoides, Synechococcus sp. PCC7335, Corallina officinalis, and Saccharomyces cerevisiae.
   55. The method of any one of the preceding embodiments, wherein the peroxidase is derived from Corallina officinalis.
   56. The method of any one of the preceding embodiments, wherein the peroxidase is derived from Aphanocapsa montana.
   57. The method of any one of the preceding embodiments, wherein the peroxidase is derived from Curvularia inaequalis.
   58. The method of any one of the preceding claims, wherein the peroxidase is produced in a host cell microorganism.
   59. The method of any one of the preceding embodiments, wherein the host cell microorganism is selected from Pichia pastoris, Aspergillus niger, or Escherichia coli.
   60. The method of any one of the preceding embodiments, wherein expression of the peroxidase produced in the host cell microorganism is increased by about 1.5-fold, 2-fold, 3-fold, 4-fold, 5-fold, 6-fold, 7-fold, 8-fold, or 10-fold, e.g., over a peroxidase produced in its native host.
   61. The method of any one of the preceding embodiments, wherein the amino acid sequence of the peroxidase is selected from an amino acid sequence listed in Table 2.
   62. The method of any one of the preceding embodiments, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from the list in Table 2.
   63. The method of any one of the preceding embodiments, wherein the peroxidase is a sequence selected from any one of SEQ ID NOs. 1-50.
   64. The method of any one of the preceding embodiments, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from SEQ ID NOs: 1-50.
   65. The method of any one of the preceding embodiments, wherein the amino acid sequence of the peroxidase has at 1, 2, 3, 4, 5, or 6 amino acid substitutions relative to an amino acid sequence selected from any one of SEQ ID NOs. 1-50.
   66. The method of any one of the preceding embodiments, wherein the small organic compound comprises 1, 2, 3, 4, 5, 6, 7, or 8 carbon atoms.
   67. The method of any one of the preceding embodiments, wherein the small organic compound comprises a ketone or aldehyde.
   68. The method of any one of the preceding embodiments, wherein the small halogenated organic compound is chlorinated, brominated, or iodinated.
   69. The method of any one of the preceding embodiments, wherein the small halogenated organic compound is brominated.
   70. The method of any one of the preceding embodiments, wherein the small halogenated organic compound comprises 1, 2, 3 halogen atoms.
   71. The method of any one of the preceding embodiments, wherein the small halogenated organic compound comprises 1, 2, 3 bromine atoms.
   72. The method of any one of the preceding embodiments, wherein the small halogenated organic compound comprises an acetone moiety.
   73. The method of any one of the preceding embodiments, wherein the small halogenated organic compound comprises dibromoacetone, bromoacetone, bromopentanedione, bromoform, or tribromoacetone.
   74. The method of any one of the preceding embodiments, wherein the small halogenated organic compound comprises 1,1-dibromoacetone, bromoacetone, 3-bromo-2,4-pentanedione, bromoform, 1,1,3-tribromoacetone, or 1,1,1-tribromoacetone.
   75. The method of any one of embodiments 40-73, wherein the conditions sufficient to product a small halogenated organic compound comprise a pH between 4-10.
   76. The method of any one of embodiments 40-74, wherein the conditions sufficient to product a small halogenated organic compound comprise an ionic strength between 0.1 mM to 4 M.
   77. The method of any one of the preceding embodiments, further comprising evaluating the small halogenated organic compound produced, e.g., by an analytical technique.
   78. The method of embodiment 77, wherein the analytical technique comprises WPLC, GC-MS, NMR.
   79. A method for increasing expression of a peroxidase in a host cell or host microorganism.
   80. A reactor for the continuous production of a small halogenated organic compound comprising:
(i) a reaction chamber;
(ii) a module for temperature control;
(iii) a peristaltic pump; and
(iv) a module for housing a catalyst.
   81. A reactor for the continuous production of a small halogenated compound of claim 79 further comprising:
(i) a device for regulating the internal atmosphere;
(ii) an impeller for stirring the reaction mixture;
(iii) a plurality of sensors for monitoring the reaction.
   82. The method of any of the preceding embodiments for modulating the production of a small halogenated organic compound, further comprising providing the peroxide source (e.g., peracetic acid) and a halogen source (e.g., a bromine source, a chlorine source, or an iodine source, e.g., a chlorinated salt such as KCl or NaCl) to the peroxidase (e.g., a VCPO).
   84. The method of any of the preceding embodiments for modulating the production of a small halogenated organic compound, further comprising:
(i) providing a peroxide source, e.g. PAA, and a chlorine source to a VCPO to generate a hypochlorite anion or its conjugate acid;
(ii): providing the hypochlorite anion to an amine to generate a chlorinated amine, e.g. NH₂C₁;
(iii) reacting the chlorinated amine with a iodide anion to generate a hypoiodite anion or its conjugate acid; and
(iv) reacting the hypoiodite anion with the small organic compound dichloroacetic acid to generate the small halogenated organic compound product dichloroiodomethane.
   85. The method of embodiments 83-84 for modulating the production of a small halogenated organic compound wherein the conversion rate of hypohalite anion and excess iodide anion to diatomic iodine I₂ and triiodide anion I₃⁻ at pH between 0-5 is reduced between 10%-99% over the method comprising the enzymatic reaction of H₂O₂ and VHPO of claim 1 at pH between 0-5.
   86. The method of embodiments 83-84 for modulating the production of a small halogenated organic compound wherein the conversion rate of hypohalite anion and excess iodide anion to diatomic iodine I₂ and triiodide anion I₃⁻ at pH 7 is reduced between 10%-99% over the method comprising the enzymatic reaction of H₂O₂ and VHPO at pH 7.
   87. The method of any of embodiments 83-84 for modulating the production of a small halogenated organic compound wherein the conversion rate of hypohalite anion and bromide anion to diatomic bromine Br₂ at pH between 0-5 is reduced between 10%-99% over the method comprising the enzymatic reaction of H₂O₂ and VHPO of claim 1 at pH between 0-5.
   88. The method of any of embodiments 83-84 for modulating the production of a small halogenated organic compound wherein the conversion rate of hypohalite anion and chloride anion to diatomic chlorine C₁₂ at pH between 0-5 is reduced between 10%-99% over the method comprising the enzymatic reaction of H₂O₂ and VHPO of claim 1 at pH between 0-5.
   89. The method of any of embodiments 83-84 for modulating the production of a small halogenated organic compound wherein the conversion rate of peracetic acid and bromide anion to hypobromite anion or its conjugate acid at pH between 0-5 is reduced between 10%-99% over the method comprising the enzymatic reaction of H₂O₂ and VHPO of claim 1 at pH between 0-5.
   90. The method of any of embodiments 83-84 for modulating the production of a small halogenated organic compound wherein the conversion rate of peracetic acid and iodide anion to hypoiodite anion or its conjugate acid at pH between 0-5 is reduced between 10%-99% over the method comprising the enzymatic reaction of PAA and VHPO of claim 1 at pH between 0-5.
   91. The method of any of embodiments 83-90 for modulating the production of a small halogenated organic compound wherein:
(i) the conversion rate of hypohalite anion and excess iodide anion to diatomic iodine I₂ and triiodide anion I₃⁻ at pH between 0-5 is reduced between 10%-99% over the method comprising the enzymatic reaction of H₂O₂ and VHPO of claim 1 at pH between 0-5;
(ii) the conversion rate of hypohalite anion and excess iodide anion to diatomic iodine I₂ and triiodide anion I₃⁻ at pH 7 is reduced between 10%-99% over the method comprising the enzymatic reaction of H₂O₂ and VHPO of claim 1 at pH 7;
(iii) the conversion rate of hypohalite anion and bromide anion to diatomic bromine Br₂ at pH between 0-5 is reduced between 10%-99% over the method comprising the enzymatic reaction of H₂O₂ and VHPO of claim 1 at pH between 0-5;
(iv) the conversion rate of hypohalite anion and chloride anion to diatomic chlorine Cl₂ at pH between 0-5 is reduced between 10%-99% over the method comprising the enzymatic reaction of H₂O₂ and VHPO of claim 1 at pH between 0-5;
(v) the conversion rate of peracetic acid and bromide anion to hypobromite anion or its conjugate acid at pH between 0-5 is reduced between 10%-99% over the method comprising the enzymatic reaction of H₂O₂ and VHPO of claim 1 at pH between 0-5; or,
(vi) the conversion rate of peracetic acid and iodide anion to hypoiodite anion or its conjugate acid at pH between 0-5 is reduced between 10%-99% over the method comprising the enzymatic reaction of PAA and VHPO of claim 1 at pH between 0-5,
   or a combination thereof.
   92. The method of any of embodiments 83-84 for modulating the production of a small haogenated organic compound wherein the conversion rate of hypochlorite and hypoiodite to iodite anion IO₂⁻ and iodate anion IO₃⁻ at pH between 7-14 is reduced between 10%-99% over the method comprising the enzymatic reaction of H₂O₂ and VHPO of claim 1 at pH between 7-14.
   93. The method of any of embodiments 83-84 for modulating the production of a small halogenated organic compound wherein the conversion rate of hypobromite and hypoiodite to iodite anion IO₂⁻ and iodate anion IO₃⁻ at pH between 7-14 is reduced between 10%-99% over the method comprising the enzymatic reaction of H₂O₂ and VHPO at pH between 7-14.
   94. The method of any of embodiments 83-84 for modulating the production of a small halogenated organic compound wherein the conversion rate of hypoiodite and hypoiodite to iodite anion IO₂⁻ and iodate anion IO₃⁻ at pH between 7-14 is reduced between 10%-99% over the method comprising the enzymatic reaction of H₂O₂ and VHPO of claim 1 at pH between 7-14.
   95. The method of any of embodiments 83-84 for modulating the production of a small halogenated organic compound wherein the conversion rate of hypohalite and hypoiodite to iodite anion IO₂⁻ and iodate anion IO₃⁻ at pH between 7-14 is reduced between 10%-99% over the method comprising the enzymatic reaction of H₂O₂ and VHPO of claim 1 at pH between 7-14.
   96. The method of any of embodiments 83-84 for modulating the production of a small halogenated organic compound wherein:
(i) the conversion rate of hypochlorite and hypoiodite to iodite anion IO₂⁻ and iodate anion IO₃⁻ at pH between 7-14 is reduced between 10%-99% over the method comprising the enzymatic reaction of H₂O₂ and VHPO of claim 1 at pH between 7-14;
(ii) the conversion rate of hypobromite and hypoiodite to iodite anion IO₂⁻ and iodate anion IO₃⁻ at pH between 7-14 is reduced between 10%-99% over the method comprising the enzymatic reaction of H₂O₂ and VHPO at pH between 7-14; or,
(iii) the conversion rate of hypoiodite and hypoiodite to iodite anion IO₂⁻ and iodate anion IO₃⁻ at pH between 7-14 is reduced between 10%-99% over the method comprising the enzymatic reaction of H₂O₂ and VHPO at pH between 7-14, or a combination thereof.
   97. The method of any of embodiments 82-96 for modulating the production of a small halogenated organic compound wherein the k_{cat} for the method comprising the enzymatic reaction of PAA and VCPO is between 10%-500% higher than the k_{cat} for the method comprising the enzymatic reaction of H₂O₂ and VHPO.
   98. The method of any of embodiments 82-97 for modulating the production of a small halogenated organic compound wherein the k_{cat} for the method comprising the enzymatic reaction of PAA and VCPO is between 25%-250% higher than the k_{cat} for the method comprising the enzymatic reaction of H₂O₂ and VHPO.
   99. The method of any of embodiments 82-98 for modulating the production of a small halogenated organic compound wherein the k_{cat} for the method comprising the enzymatic reaction of PAA and VCPO is between 50%-150% higher than the k_{cat} for the method comprising the enzymatic reaction of H₂O₂ and VHPO.
   100. The method of any of embodiments 82-99 for modulating the production of a small halogenated organic compound wherein the k_{cat} for the method comprising the enzymatic reaction of PAA and VCPO is between 75%-125% higher than the k_{cat} for the method comprising the enzymatic reaction of H₂O₂ and VHPO.
   101. The method of any of embodiments 82-100 for modulating the production of a small halogenated organic compound wherein the k_{cat} for the method comprising the enzymatic reaction of PAA and VCPO is between 80%-120% higher than the k_{cat} for the method comprising the enzymatic reaction of H₂O₂ and VHPO.
   102. The method of any of embodiments 82-101 for modulating the production of a small halogenated organic compound wherein the k_{cat} for the method comprising the enzymatic reaction of PAA and VCPO is between 90%-110% higher than the k_{cat} for the method comprising the enzymatic reaction of H₂O₂ and VHPO.
   103. The method of any of embodiments 82-102 for modulating the production of a small halogenated compound wherein the product is dichloroiodomethane.
   104. The method of any of embodiments 82-102 for modulating the production of a small halogenated compound wherein the product is dibromochloromethane.
   105. The method of any of embodiments 82-102 for modulating the production of a small halogenated compound wherein the products are dichloroiodomethane and dibromochloromethane.

### EXAMPLES

The present disclosure is further described in detail by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compositions, methods, and devices of the present disclosure and practice the claimed methods. The following working examples specifically point out various aspects of the present disclosure and are not to be construed as limiting in any way the remainder of the disclosure.

### Example 1: Heterologous Expression and Purification of Exemplary Vanadium Haloperoxidases from Escherichia coli

Exemplary vanadium haloperoxidases (VBPO) genes overexpressed in E. coli according to the following procedure. The full nucleotide sequence of various VBPOs were chemically synthetized using a commercial vendor (GeneScript) and cloned into the pET-28a(+) vector, suitable for E. coli protein expression. The resulting proteins were designed to contain a HRV 3C protease recognition signal at the N-terminus and a C-terminal 6x-His tag to enable affinity purification. After culturing the E. coli cells using standard techniques, the recombinant VBPOs were purified to homogeneity using a two-step protocol including an affinity column-based protein purification step (HisTrap FF Crude) and second a size-exclusion step (Amicon™ Ultra Centrifugal Filter Units 100 kDa), and then used without further purification.

### Example 2: Purification and Kinetic Characterization of an Exemplary Vanadium Haloperoxidase from Corallina officinalis

The purification of a VBPO naturally produced in the seaweed Corallina officinalis (CoVBPO) is provided in this example. After harvesting the seaweed, a crude extract was prepared by flash-freezing in liquid nitrogen and grinding to fine particles. Next, a buffer solution of Tris-sulfate 0.1 M, pH=8.5, was added in a 1:5 proportion. After centrifugation, the supernatant constitutes the crude extract. The CoVBPO was purified in a 3-step protocol summarized in Table 3. Analysis by SDS-PAGE indicated that the final enzyme preparation was over 90% pure.

TABLE 3
Summary of enzyme purification

| | | Total | Total | Specific |
|---|---|---|---|---|
| Purification | Volume | Enzyme | Enzyme | Enzyme |
| Protocol | (ml) | (mg) | activity (IU) | activity (IU/mg) |
| Crude supernatant | 720 | 260 | 78 | 0.30 |
| Heat Treatment | 695 | 130 | 125 | 0.94 |
| Ammonium sulfate | 45 | 32 | 124 | 3.90 |
| Filtration (100 | 20 | 11 | 78. | 7.31 |
| kDa) | | | | |

The CoVBPO was then studied to determine various kinetic parameters based on a 3D fitting using the Cleland equation, using compositions of CoVBPO in MOPS buffer pH=7.0 and MES buffer pH=6.5 at ionic strengths 0.2 and 2.0 M respectively.

All kinetic measurements were performed based on analysis of initial velocities of an exemplary reaction, namely the dearylation of aminophenyl fluorescein (APF) to fluorescein (Archer et al. 2019). Fluorescence was measured using a Photon Technology International (PTI) QuantaMaster fluorometer. Excitation wavelength was set to 490 nm and emission recorded at 515 nm by parallel photomultiplier detection systems; Lamp output was 74 W).

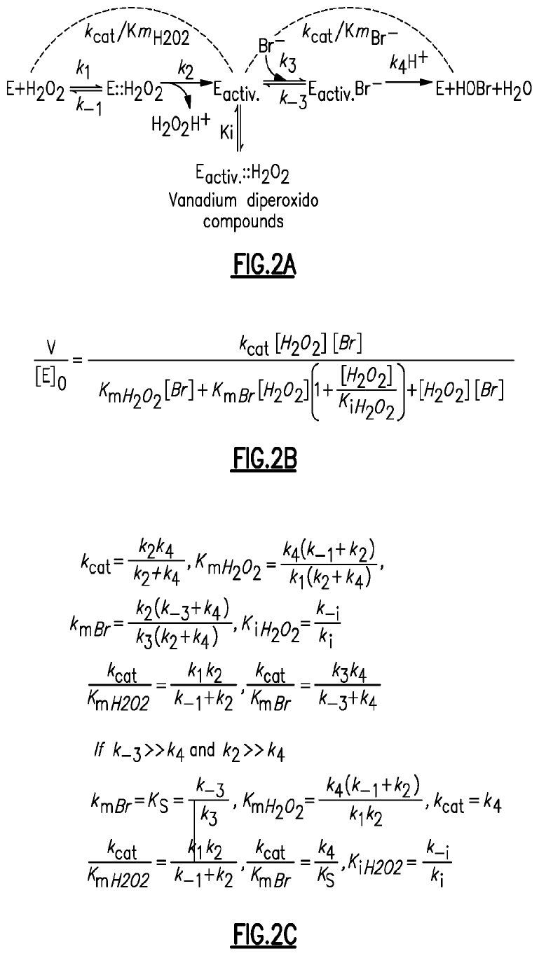

As shown in FIGS. 2A-2C, in the ping pong bi-bi reaction system, the first product Pi (H₂O) is released after the binding of the first substrate (hydrogen peroxide) onto the enzyme molecule and before the binding of the second substrate (bromide). The surface fitting of the experimental kinetic data was performed by means of the non-linear multiparametric equation shown in FIG. 2B. The non-linear multiparametric equation, where substrate inhibition results in the formation of one dead-end complex, were formulated using the King-Altman method (see, e.g., FIG. 2C). All fitting procedures, as well as the goodness of the fit, were performed employing OriginPro 2021 version; a non-weighted least-squares test was used as the convergence criterion in all cases. Tables 4 and 5 summarize the resulting parameters determined through this analysis at both ionic strengths tested.

TABLE 4
Exemplary kinetic parameters for CoVBPO for ionic strength 2M

| Parameter | Value | Standard Error | t-Value |
|---|---|---|---|
| k_{cat} | 18.3 | 0.24 | 75 |
| Km_{Br} | 8.21E−4 | 4.5E−5 | 18 |
| Km_{H2O2} | 1.28E−5 | 5.1E−7 | 25 |
| ki _{H2O2} | 1.14E−4 | 1.0E−5 | 11 |
| k_{cat}/Km_{Br} | 2.23E4 | | |
| k_{cat}/Km_{H2O2} | 1.43E6 | | |

TABLE 5
Exemplary kinetic parameters for CoVBPO for ionic strength 0.2M

| Parameter | Value | Standard Error | t-Value |
|---|---|---|---|
| k_{cat} | 30.2 | 0.48 | 63 |
| Km_{Br} | 0.011 | 2.7E−4 | 40 |
| Km_{H2O2} | 5.84E−5 | 1.7E−6 | 35 |
| ki _{H2O2} | 5.57E−4 | 3.0E−5 | 19 |
| k_{cat}/Km_{Br} | 2.83E3 | | |
| k_{cat}/Km_{H2O2} | 5.18E5 | | |

The results indicate that the CoVBPO may proceed via a ping-pong bi-bi mechanism, which involves hydrogen peroxide substrate dead-end inhibition. In low ionic strength (0.2M), an increased turnover parameter (k_{cat}) was observed. Additionally, the estimated values of parameters Km_{H2O2} and Km_{Br} showed a 4.5 times higher affinity of VBPO to hydrogen peroxide and a more than one order magnitude (12 times) increased affinity to bromide in high ionic strength, respectively. The inhibition constant for hydrogen peroxide follows a similar pattern as the Km_{H2O2}.

### Example 3: Effect of pH and Temperature on Exemplary VBPOs

Using the CoVBPO enzyme, the pH dependence of Michaelis-Menten kinetics were examined in high and low ionic strength media. The aims were: i) to explain the binding mechanism of the second substrate (bromide, Br⁻), an aspect of the reaction that remained poorly understood and hampered utilization of VHPO; and ii) to reduce the corresponding K_{mBr-} through engineering of the reaction medium.

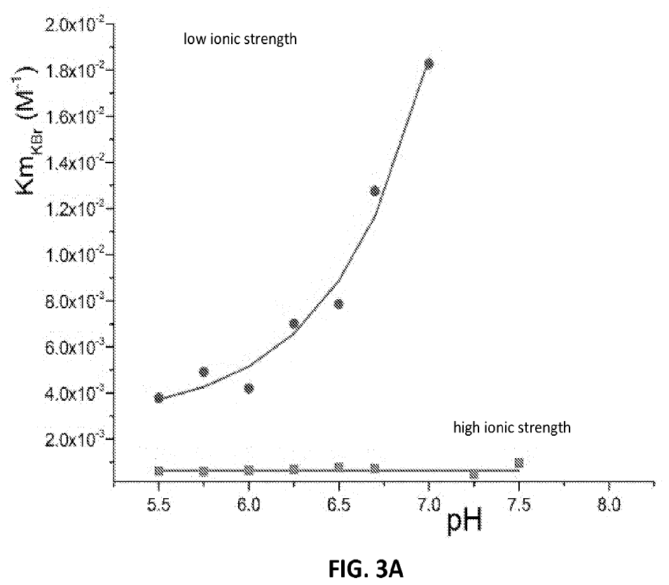

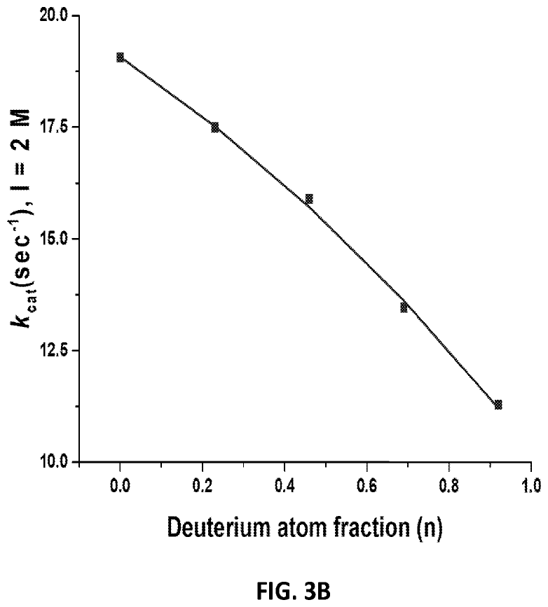

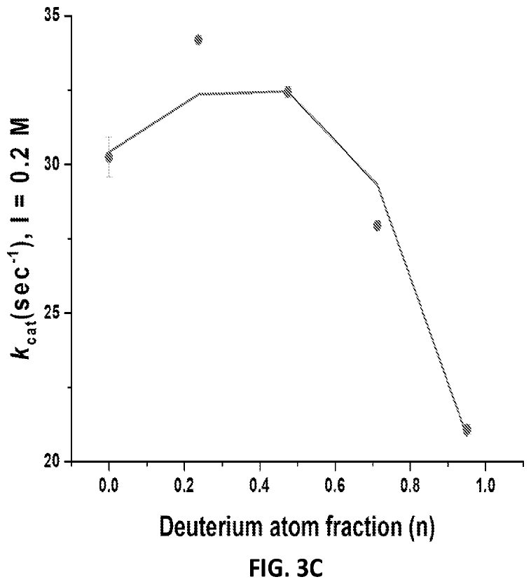

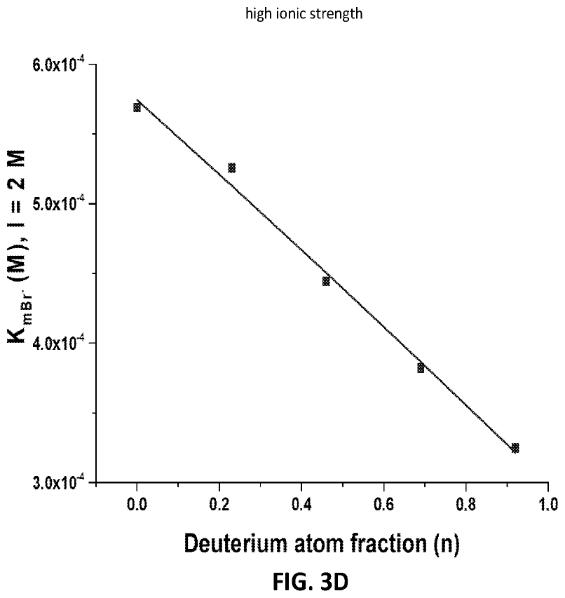

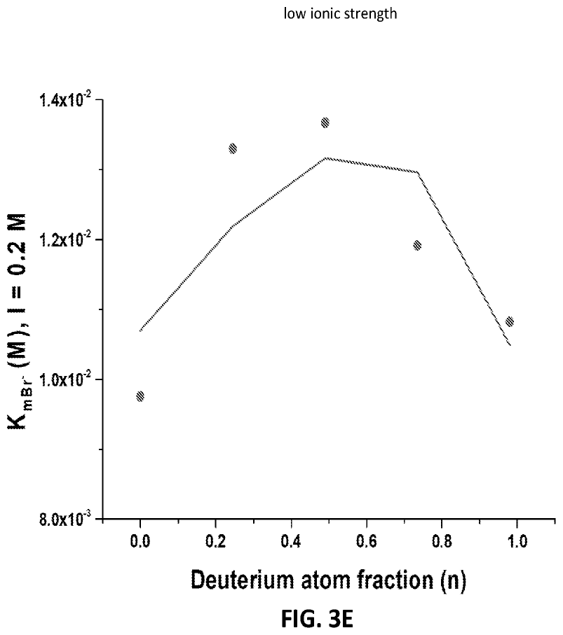

The presence of high ionic strength In the solution demolishes ionic interactions developing in the solvent-accessible amino acids of the enzyme. In low ionic strength, the affinity for Br⁻ decreases (inceasing KmBᵣ) at higher pH values (FIG. 3A).

### Example 4: Characterization of Halogenated Organic Compounds Produced by an Exemplary VBPO

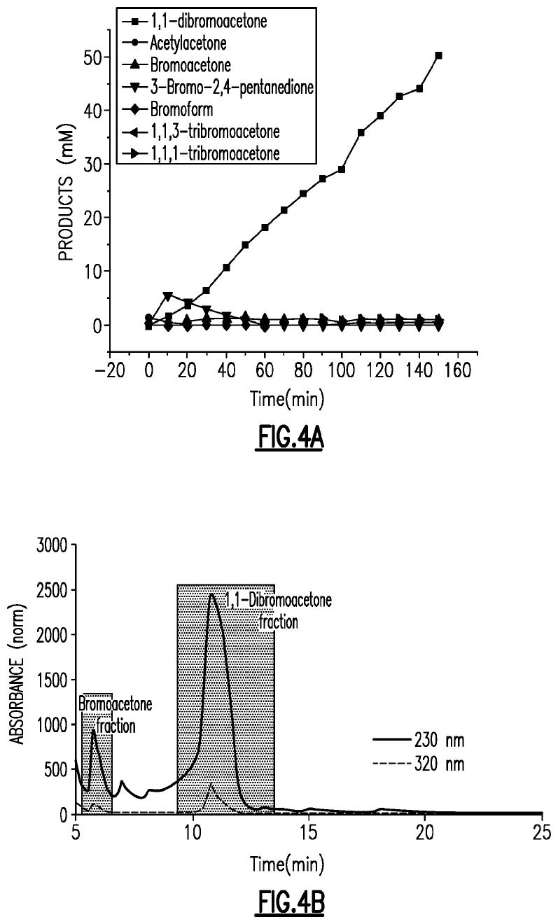

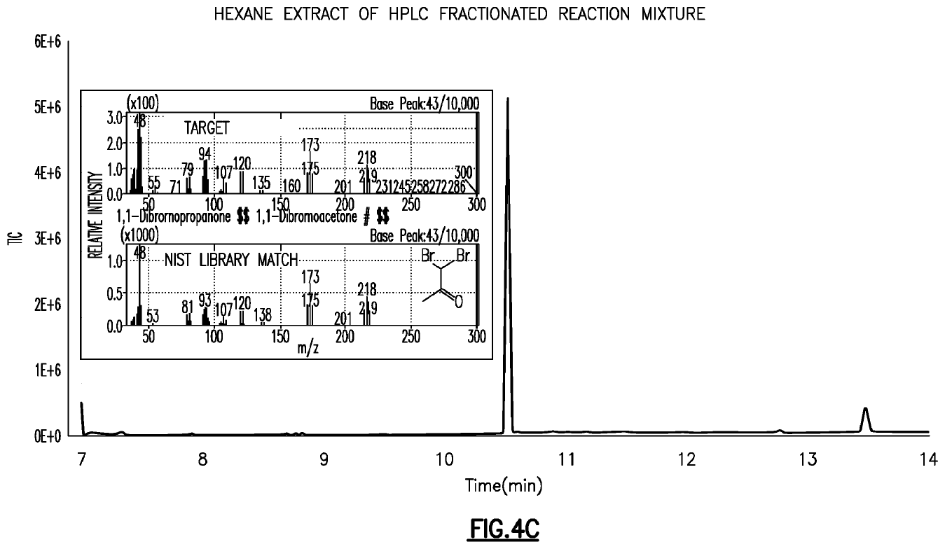

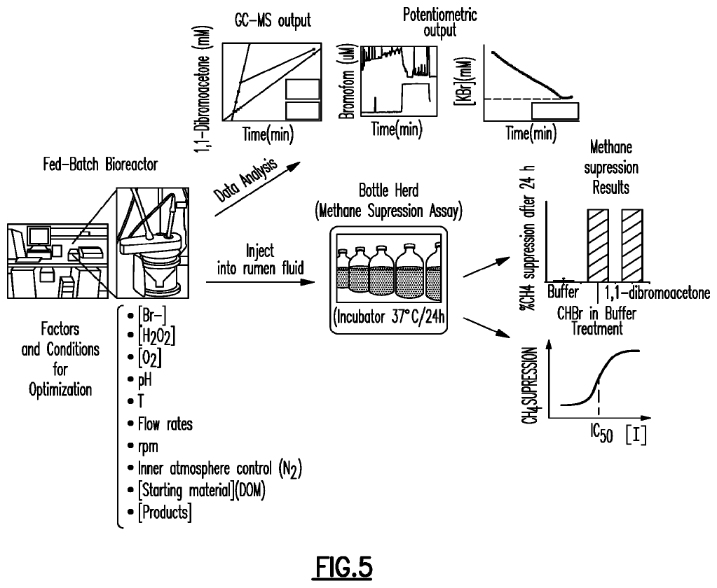

This example describes how certain halogenated compounds can be favored for production over others by carefully modulating the VBPO reaction conditions. Acetyl acetone was incubated with VBPO at a near constant concentration in a fed-batch bioreactor. VHPO substrates H₂O₂ and KBr were added concomitantly to the fed-batch bioreactor in a controlled manner via a peristaltic pump. The concentration of the reaction product is monitored by removing aliquots of the reaction over time and analyzing the reaction profile by GC-MS. As shown in FIG. 4A, the main product produced was 1,1-dibromoacetone, which accumulated to 50 mM. Small amounts of other halogenated compounds were produced, including bromoacetone, 3-bromo-2,4-pentanedione, bromoform, 1,1,3-tribromoacetone, and 1,1,1,-tribromoacetone. Napthalene was used as an internal standard to determine halogenated analyte concentrations.

The products produced were purified in batch mode using an HPLC method; an exemplary HPLC chromatogram is shown in FIG. 4B. Following HPLC fractionation, the 1,1,-dibromoaceone was extracted with n-hexane and further analyzed by GC-MS. The identity of the n-hexane extracted product was made by comparison of its MS spectrum with the MS spectrum of a standard from a NIST library (FIG. 4C).

### Example 5: Inhibition of Methane Production by Exemplary Small Halogenated Organic Compounds in Rumen-Derived Microbial Communities

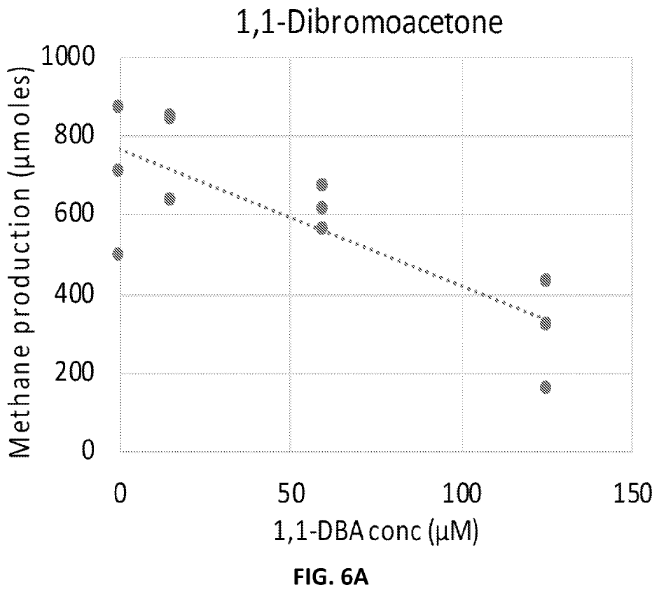

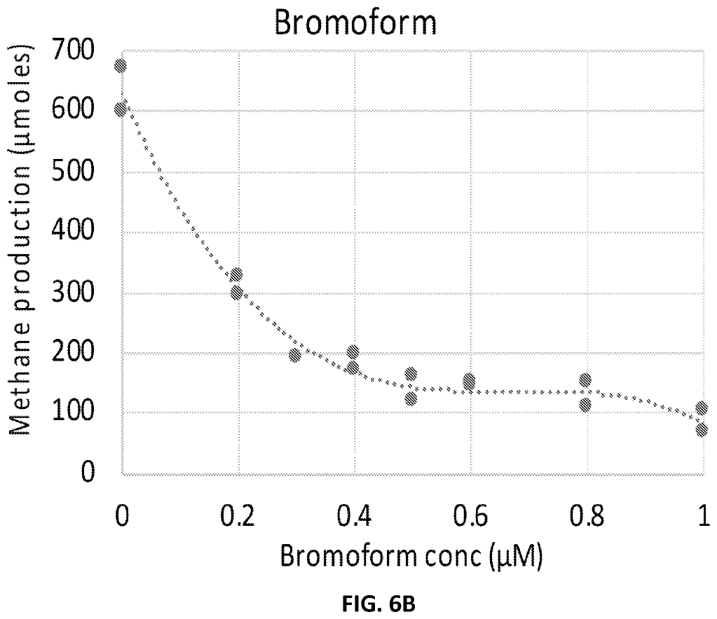

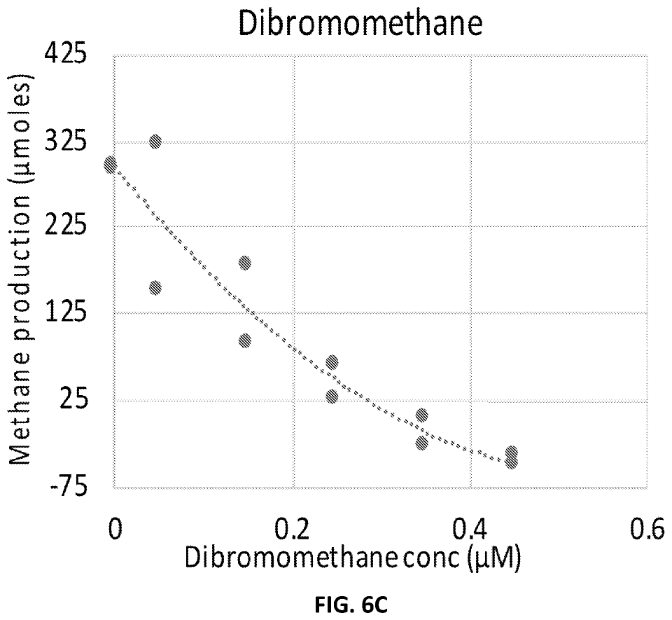

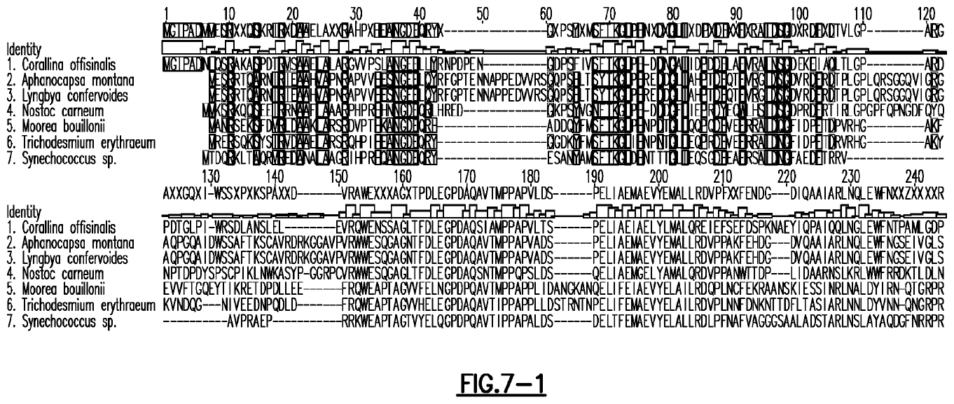

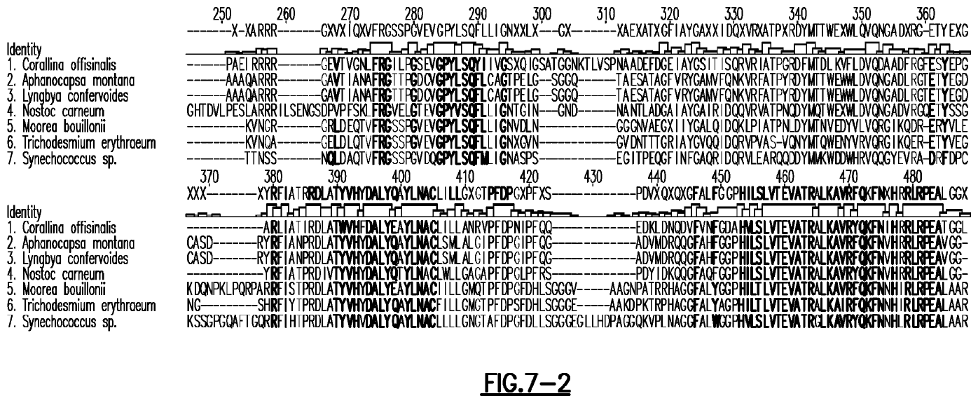

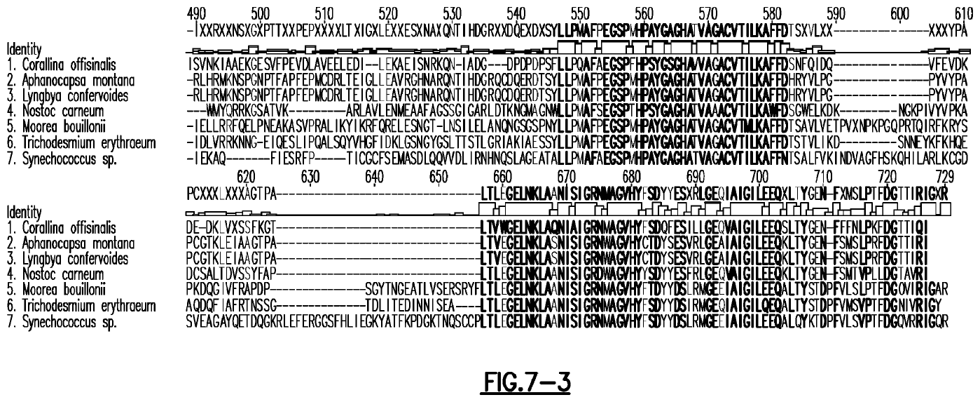

FIG. 5 illustrates the production and testing process of small organic halogenated compounds for inhibition of methanogenesis. In this example, the small organic halogenated compounds produced by VBPO are incubated in a microbial culture system derived from rumen fluid obtained from dairy cattle, to determine their relative abilities at inhibiting methane production. Briefly, 1,1-dibromoacetone, co-product bromoform and derivative dibromomethane, were separately added to the culture systems. The rate of methane production was monitored in each reaction (FIGS. 6A-6C).

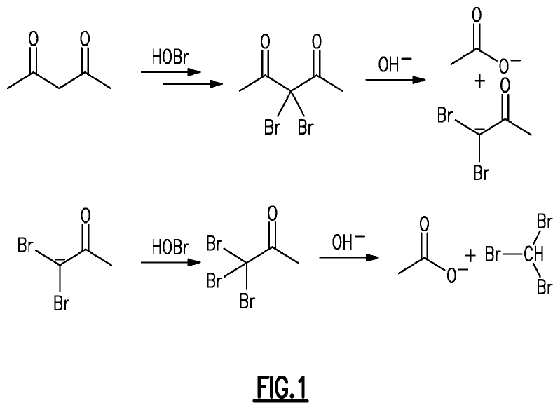

As shown in FIGS. 6A-6C, 1,1-dibromoacetone was the least effective of the compounds screened for inhibiting methane production. 1,1-dibromoacetone provided a 50% suppression of methane production at 125 uM in the rumen-derived microbial communities tested, compared with bromoform, which showed a 50% suppression of methane production at 0.20 uM, and dibromomethane, which showed 50% suppression of methane production at 0.15 uM.

### Example 6: Heterologous Production of cchVBPO in Escherichia coli

This example describes the heterologous expression of Chrondrus crispus VBPO (cchVBPO) in E. coli, namely the inoculation and culture of the model microorganism in a bioreactor, induction of cchVBPO production, protein extraction, purification, and biochemical characterization.

### Inoculation and Induction

Briefly, 4×5 ml of LB media containing Kanamycin (4.0 μl, maintained at about 0° C.) was inoculated in a culture tube (15 ml) with a single colony from a Kanamycin+agar plate (or 5-10 μl from a glycerol stock). The inoculum was subsequently incubated at 37° C. with shaking at 250 rpm, and the OD₆₀₀ was measured to be about between 0.4-0.6 (200 μl in the multi-well plate) after overnight, i.e., 15-18 h. Then the cultures (4% inoculation/20 ml) were added to a 0.5-L (preheated in 37° C. medium) bioreactor containing 400 pd Kanamycin ([Kanamycin]=40 g/ml; stock solution is 50 mg/ml) and shaken at 200 rpm at 37° C. until the OD₆₀₀ was approximately 0.6-0.7. The OD₆₀₀ was monitored during growth by removing aliquots aseptically. When the OD₆₀₀=0.6-0.7, the culture was cooled to 18° C. and 0.1 mM IPTG (0.5 ml, from stock 100 mM IPTG) was added. 3-ml samples were taken to use as t=0 h (uninduced). The culture was incubated overnight at 18° C. and stirred at 200 rpm under oxygen supply. Then it was centrifuged at 10,000 rpm for 10 min at 4° C. in pre-weighted 500 ml centrifugation tubes (wherein the pellet was drained by inversion and the excess medium was tapped onto a paper towel). The sample was subsequently maintained on ice.

### Extraction and Preparation

The pellet from previous step was resuspended in BugBuster® Master (commercially available from EMD Millipore) using 5 ml of 1× reagent per gram of wet cell paste. 5 grams of the cell paste was mixed gently by light vortex with 22.5 ml Borate buffer 20 mM, pH=8.0, 30 mM imidazole [binding buffer]. 2.5 ml 10× BugBuster® and one pill of protease inhibitor was subsequently added to the mixture. Protease inhibitors were optionally included, due to their compatibility with BugBuster® Master Mix. Serine protease inhibitors were avoided if the target protein was to be treated with Thrombin, Factor Xa, or Recombinant Enterokinase. Cysteine protease inhibitors were if the target protein was to be treated with HRV 3C. Although purification removes active inhibitors, dialysis or gel filtration would be recommended before cleavage. The cell suspension was incubated on a shaking platform or rotating mixer at a slow setting for 20 min at room temperature. The extract should not be viscous after incubation. Then the suspension was centrifuged in 4° C. for 15 min at 75,600 g (JA-25.50, 8×30 ml) to remove insoluble cell debris. The pellet was then optionally washed again with 5 ml binding buffer for 5 min and the centrifugation repeated. The steps above were repeated for the pre-induced sample. 0.5 ml of the induced and pre-induced samples were saved, and the induced supernatant was transferred to a new tube.

### Purification

A HisTrap FF Crude 5 ml column was assembled and the storage buffer was removed with 5 CV of binding buffer (20 mM boric acid, pH 8+30 mM imidazole). The column was then equilibrated with 5 CV binding buffer (20 mM boric acid, pH 8.0+30 mM imidazole) and loaded with the sample from the previous step. Next the sample was washed with 6 CV binding buffer, or until UV₂₈₀ reached a stable value, and finally eluted with elution buffer (20 mM boric acid, pH 8.0+300 mM imidazole) to yield the cchVBPO eluate.

### Characterization

The protein concentration of the eluate yielded from the last step was determined by Bradford Assay. In short, to a 250-μl well plate at room temperature, Bradford reagent and 5 μl of the protein sample was incubated for 5-10 min and OD₆₀₀ was measured. Total protein concentration was determined against a calibration curve with BSA standards (0.125 mg/ml-2 mg/ml). Tables 6 and 7 below give the Bradford calibration curve of known BSA standards and the determined protein concentrations of the pre-induced and purified, induced samples

TABLE 6
Bradford calibration curve of BSA standards used to measure the
total protein concentration of induced and pre-induced samples
from the heterologous cchVBPO expression system in E. coli.

| | | | | | | | | |
|---|---|---|---|---|---|---|---|---|
| mg/ml | 0 | 0.125 | 0.25 | 0.5 | 0.75 | 1.0 | 1.5 | 2 |
| Volume of added | 5 μl | 5 | 5 | 5 | 5 | 5 | 2.5 | 2.5 |
| protein in μl | H₂O₂ | | | | | | | |
| OD₆₀₀ | 0 | 0.105 | 0.198 | 0.411 | 0.557 | 0.645 | | |

| | |
|---|---|
| Calibration curve | y = a* x + b, a = 0.6057, b = 0.0563 |

TABLE 7
The total protein concentration of the pre-induced and purified,
induced samples from the heterologous cchVBPO
expression system in E. coli.

| | | | Total Vol/ |
|---|---|---|---|
| Sample | μl added | mg/ml | Total mg |
| Pre-Induction | 2.5 | 0.758 | — |
| sample | | | |
| After Induction | 2.5 | 2.716 | 25/67.9 |
| sample (Initial) | | | |
| Unbound sample | 2.5 | 2.495 | 27/67.3 |
| Wash sample | 2.5 | 0.022 | 30/0.66 |
| Elution sample 2 | 5 | 0.2303 | 3.5/0.804 |

Sum = 0.804 mg or 1.61 mg/L

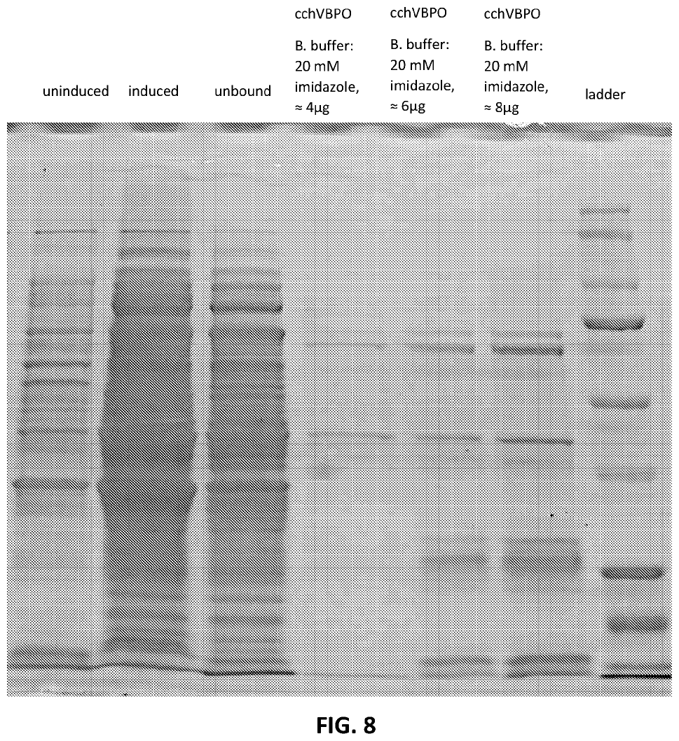

SDS-PAGE electrophoresis was also employed to verify that the eluate contained cchVBPO protein as illustrated in FIG. 8. Uninduced, induced, unbound, 4 μg cchVBPO borate buffer with 20 mM imidazole buffer, 6 μg cchVBPO in borate buffer with 20 mM imidazole, and 8 μg cchVBPO borate buffer with 20 mM imidazole, were compared to a ladder as shown. The bromoperoxidase activity was determined by APF assay (490 nm/515 nm). In brief, to a 3 ml curvette 2930 ml buffer containing 30 μl KBr (18 mM)+5 μl APF (2 μM)+5 μl VBPO (containing Na₃VO₄)+30 μl hydrogen peroxide (250 μM) were mixed. The reaction was commenced upon stirring with the addition of VBPO at 25° C. Bromoperoxidase activity was assessed at 490 nm/515 nm.

### Example 7: Heterologous Production of synVBPO in Escherichia coli

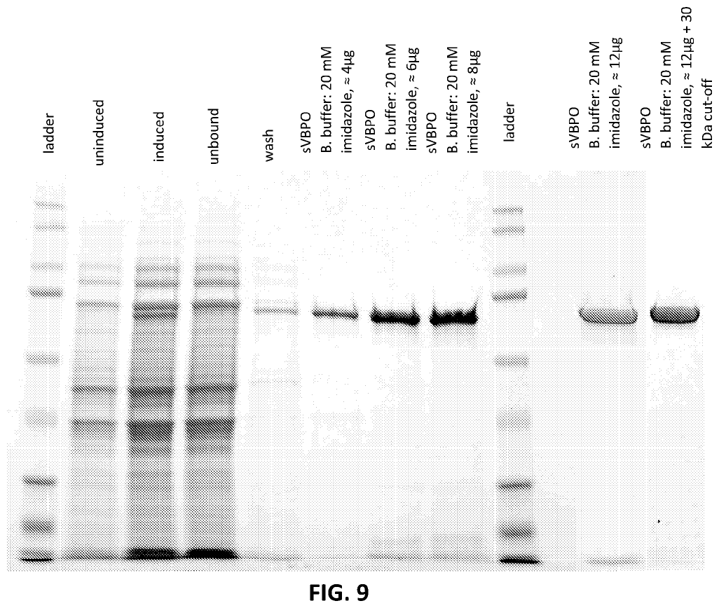

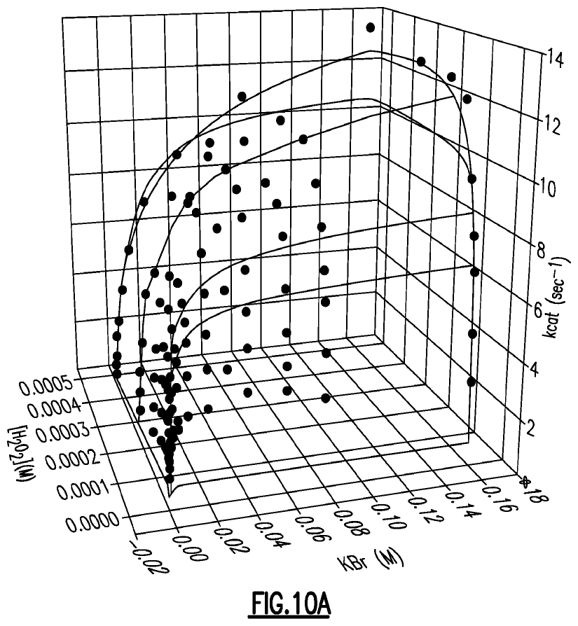

This example describes the heterologous expression of Synechococcus sp. VBPO (synVBPO) in E. coli, namely the inoculation and culture of the model microorganism in a bioreactor, induction of synVBPO production, protein extraction, purification, and biochemical characterization. Inoculation, induction, protein extraction, protein purification, and characterization (i.e., Bradford Assay, SDS-PAGE, APF Assay) were completed according to the protocols delineated in Example 6 with minor modifications as noted below. The optical density at 600 nm (OD₆₀₀) of the 5-ml sample was 0.55 17-18 h post-inoculation. After transferring the culture to a 0.5-L bioreactor under 200 rpm shaking at 37° C. the OD₆₀₀ was monitored until it reached 0.6-0.7. OD₆₀₀ was measured to be 0.312, 0.535, and 0.638, at 4 h, 5 h, and 5.5 h, respectively, at which time it was deemed sufficient to begin the induction process. 18 h after induction with 0.1 mM IPTG at 18° C. the OD₆₀₀ was determined to be 0.920. After centrifugation, the weight of the pellet was 4.162 grams. The protein was resuspended and purified employing a HisTrap FF column as previously described in Example 6. The eluate was then characterized for total protein concentration, SDS-PAGE, and haloperoxidase activity. Tables 8 and 9 below give the Bradford calibration curve of known BSA standards and the determined protein concentrations of the pre-induced and purified, induced samples. FIG. 9 is SDS-PAGE results showing ladder, uninduced, induced, unbound, 4 μg synVBPO in 20 mM borate buffer, 6 μg synVBPO in 20 mM borate buffer, 8 μg synVBPO in 20 mM borate buffer, ladder, 12 μg synVBPO in 20 mM borate buffer, 12 μg synVBPO in 20 mM borate buffer+30 kDa cutoff.

### Bradford Calibration Curve

TABLE 8
Bradford calibration curve of BSA standards used to measure the
total protein concentration of induced and pre-induced samples
from the heterologous synVBPO expression system in E. coli.

| | | | | | | | | |
|---|---|---|---|---|---|---|---|---|
| mg/ml | 0 | 0.125 | 0.25 | 0.5 | 0.75 | 1.0 | 1.5 | 2 |
| Volume of added | 5 | 5 | 5 | 5 | 5 | 5 | 2.5 | 2.5 |
| protein (μl) | | | | | | | | |
| OD₆₀₀ | 0 | 0.105 | 0.198 | 0.411 | 0.557 | 0.645 | | |

| | |
|---|---|
| Calibration curve | y = a* x + b, a = 0.6057b = 0.0563 |
| Notes | Not linear after 1 mg/ml |

TABLE 9
The total protein concentration of the pre-induced
and purified, induced samples from
the heterologous synVBPO expression system in E. coli.

| | | | Total Vol/ |
|---|---|---|---|
| Sample | μl added | mg/ml | Total mg |
| Pre-Induction | 5 | 1.313 | — |
| sample | | | |
| After Induction | 5 | 2.151 | 30/64.53 |
| sample (Initial) | | | |
| Unbound sample | 5 | 1.989 | 30/59.67 |
| Wash sample | 5 | 0.032 | 30/0.96 |
| Elution sample 1 | 5 | 0.008 | 2/0.02 |
| Elution sample 2 | 5 | 0.6 | 8/4.76 |

Sum = 4.76 mg or 9.5 mg/L

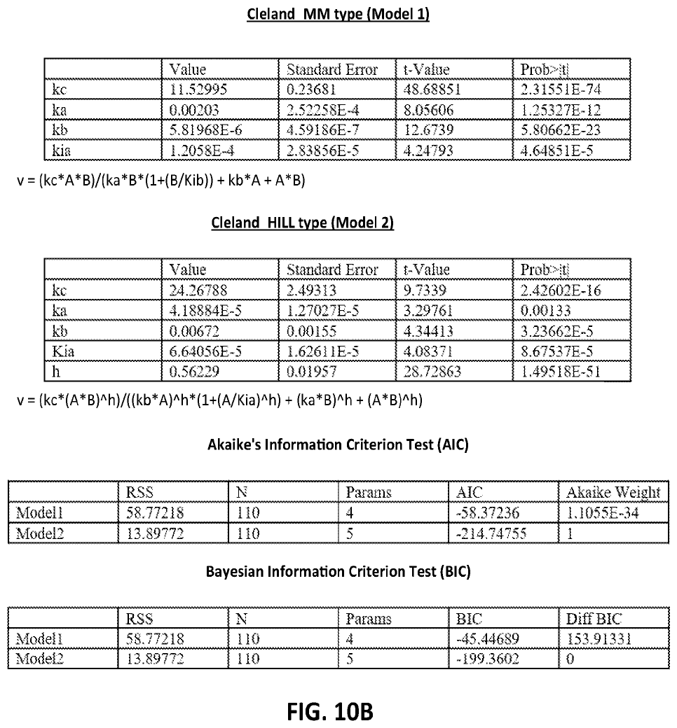

The synVBPO enzymatic kinetic surface was plotted with k_{cat} (s⁻¹) as a function of hydrogen peroxide concentration, [H₂O₂](M), and potassium bromide concentration, [KBr] (M), as illustrated in FIG. 10. Kinetic parameters, standard errors, t-values, and Prob>|t| were determined for two kinetic models: Cleland Michaelis-Menten (Model 1) and Cleland Hill type (Model 2). Akaike's Information Criterion Test (AIC) and Bayesian Information Criterion Test (BIC) yielded that the Cleland Hill type (Model 2) had the lower AIC value and lower BIC value, respectively; hence, it was inferred that the synVBPO kinetics followed a Cleland Hill-type kinetic paradigm. OriginPro (2021 version) was employed for calculation of kinetic parameters following Michaelis-Menten and Hill-type kinetics and carrying out statistical tests.

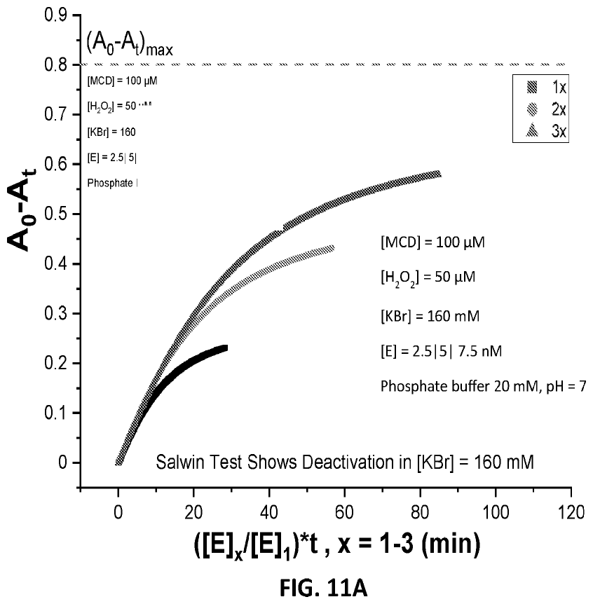

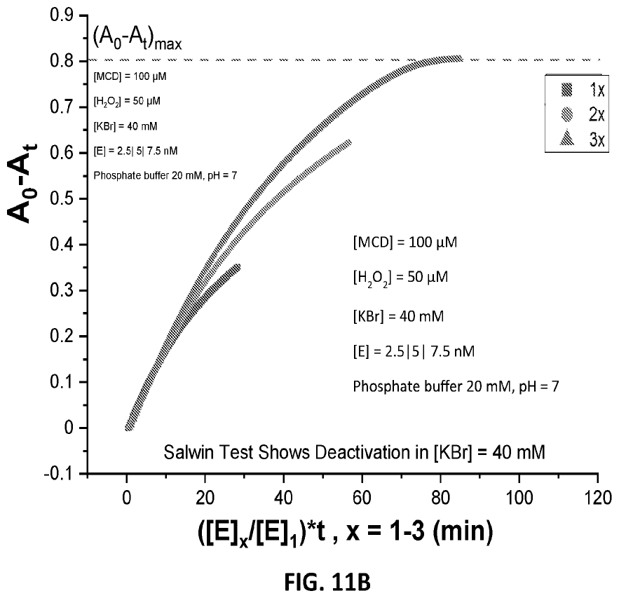

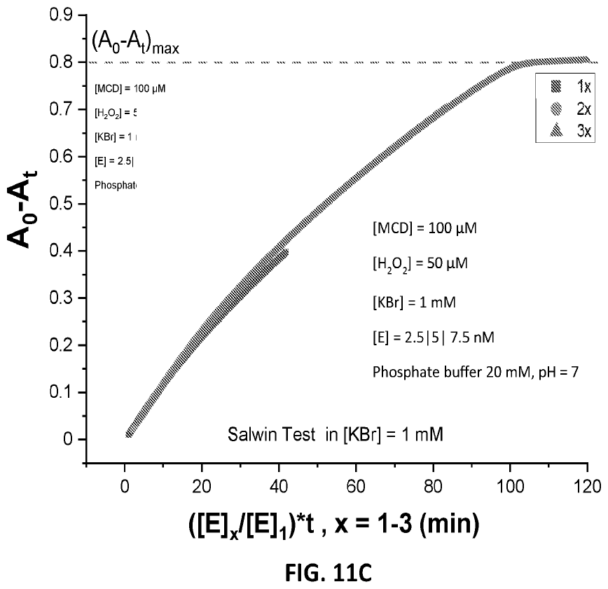

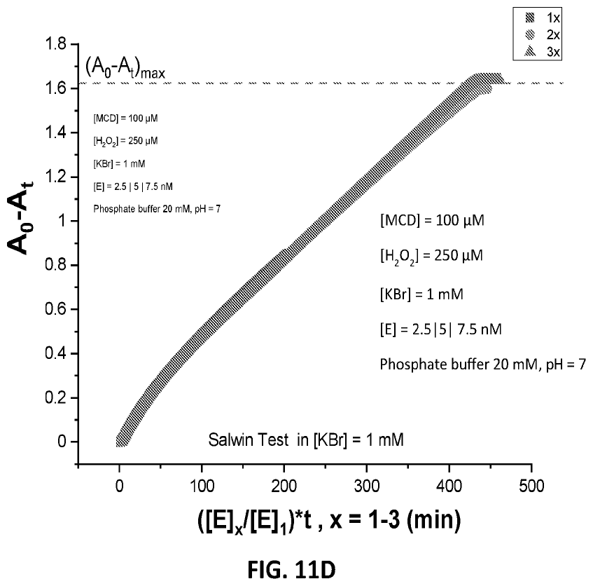

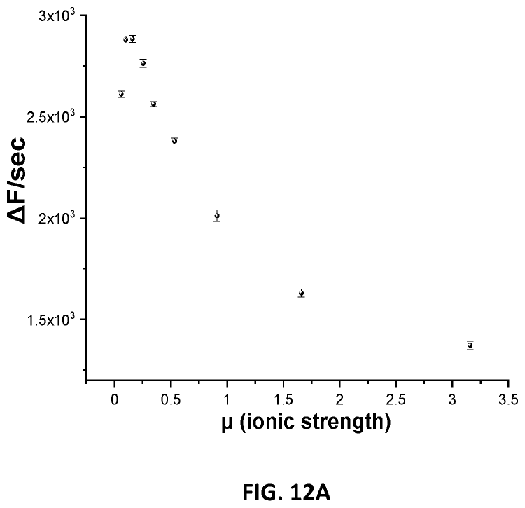

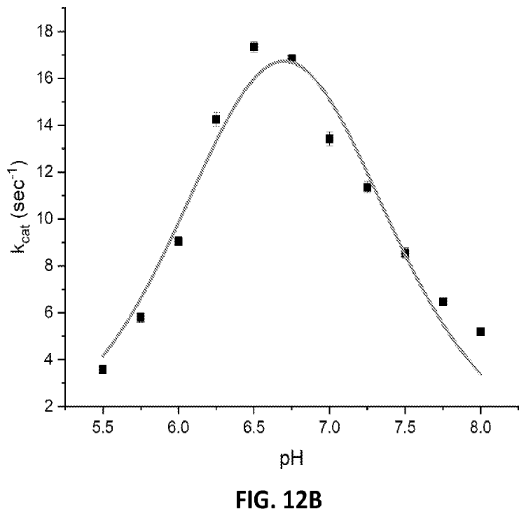

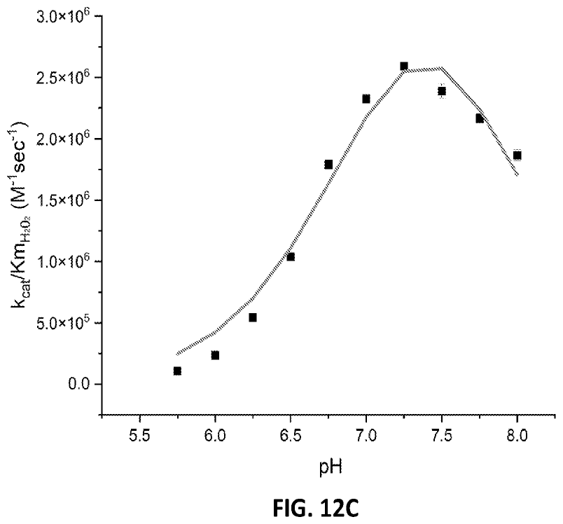

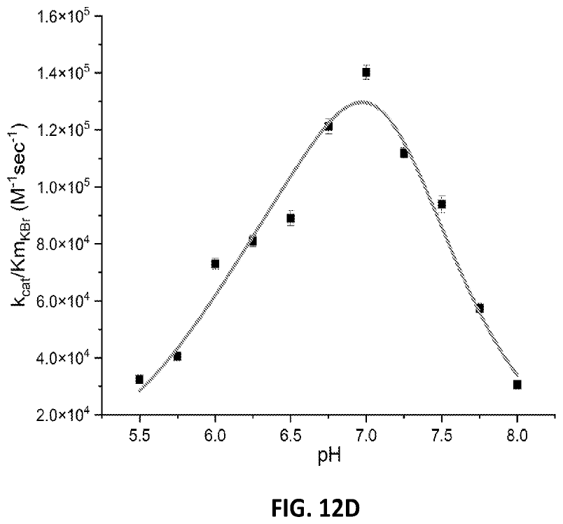

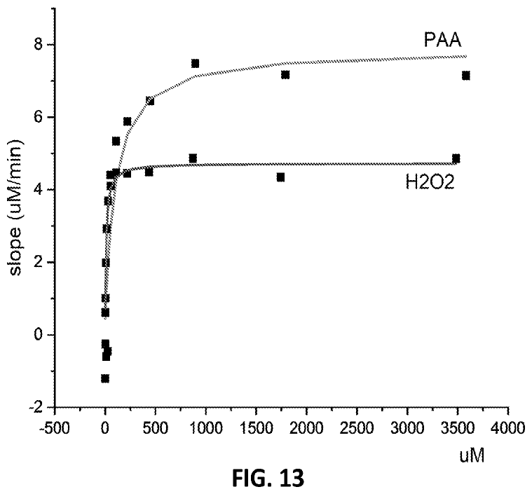

Further, the stability of synVBPO enzymatic activity at high concentrations of KBr and H₂O₂, its dependence on ionic strength, and the Michaelis-Menten parameters as a function of pH were determined as depicted in FIGS. 11-12. To interrogate the deactivating effects of KBr on synVBPO activity, Salwin tests were performed employing 100 μM MCD, at three VBPO concentrations (2.5 nM, 5 nM, and 7.5 nM) E=2.5|5| 7.5 nM in 20 mM phosphate buffer, pH 7 containing 1 mM KBr and 250 μM H₂O₂, 1 mM KBr and 50 μM H₂O₂, 40 mM KBr and 50 μM H₂O₂, and 160 mM and 50 μM H₂O₂, respectively. Successive tests at KBr concentrations of 40 mM and 160 mM demonstrated a clear deactivation of enzymatic activity at these high bromide concentrations, whereas at 1 mM KBr the enzyme retained its activity at either peroxide concentration. The relation between ionic strength and synVBPO activity is shown as the change in fluorescence per p (ionic strength) as shown in FIG. 12. Further, the kinetic parameters k_{cat} (s⁻¹), k_{cat}/Km_{H2O2} (M⁻¹s⁻¹), and k_{cat}/Km_{KBr} (M⁻¹s⁻¹) were determined employing a least-squares algorithm of the experimental data, as shown in the equation in FIG. 12.

### Example 8. PAAM Addition to ciVCPO

This Example describes a method for utilizing peracetic acid as a substrate for production of small, halogenated organic compounds in lieu of hydrogen peroxide using ciVCPO. PAAM is a readily available source of peracetic acid, consisting of 32% w/v peracetic acid and 6% w/v hydrogen peroxide. As peracetic acid is not an efficient two electron reductant, and unlike hydrogen peroxide, cannot produce oxygen in the presence of hypochlorous acid and catalytic quantities of chloride ions, hydrogen peroxide must be removed to avoid oxygen production employing the enzyme catalase, derived from bovine liver.

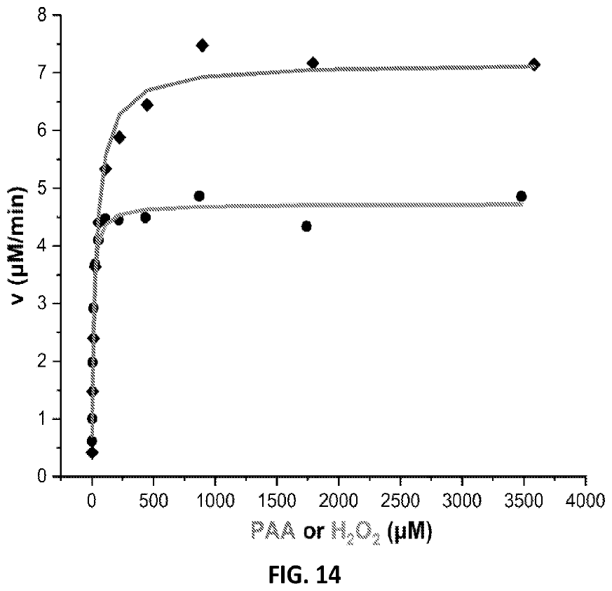

FIG. 14 shows the reaction velocity of ciVCPO in the presence of chloride ions using PAA and H₂O₂ as a substrate. The reaction velocity is fitted according to Michaelis-Menten kinetics to give v=f([PAA] or [H₂O₂]). As demonstrated by the graph, employing PAA as a substrate gives a higher vₘₐₓ and Kₘ than hydrogen peroxide for a constant concentration of NaCl of 150 mM.

### Process Designs

In each of the process designs below for production of small, halogenated organic compounds in a bioreactor, the dibromoacetone (DBA) or dichloroacetone (DCA) has been produced enzymatically, as previously described.

### Design 8.1

PAA+NaCl+DBA+ciVCPO→DBCA, where DBA is dibromoacetone and DBCA is dibromochloroacetone (at pH=7-8 and T=20-55° C.)

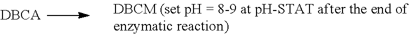

### Design 8.2

### Step 1:

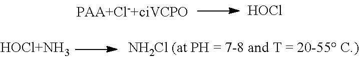

### Step 2:

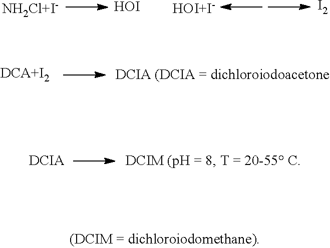

### DCIM Production in a Two-Batch System

An embodiment of the invention is described for the production of dichloriodomethane (DCIM) in a two-step batch system as delineated in the steps below according to the following conditions. In the first step, 0.1 M phosphate buffer, pH 7 containing 12 mM H₂O₂, 100 mM NaCl, 10 mM DCA at T=22° C. was added to the bioreactor with H₂O₂ added at a rate of 25 μl/min. 1 eq. H₂O₂ was added to 1.2 eq. of NH₃, i.e., CNH³ was 0.24 M, and the molar rate in of H₂O₂ into the tank was equal to the molar rate out, or C^{H2O2} was 0.2 mol/L. The reaction time was set to 120 min. The initial concentrations in the bioreactor were 100 mM NaCl, 100 μM H₂O₂, 480 μM NH₄Cl, 264 M Na₃VO₄, and 2.64 μM enzyme.

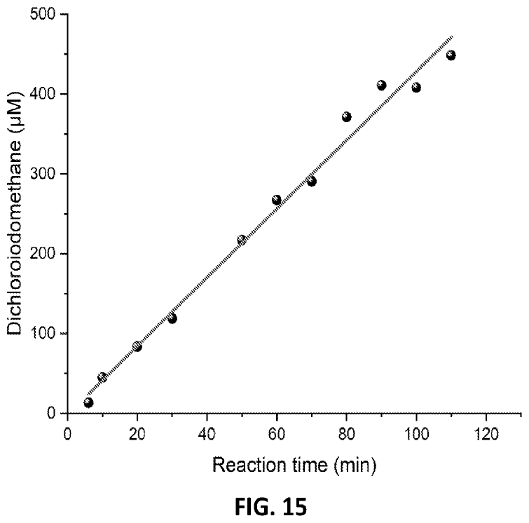

Every ten minutes 500 μl of the bioreactor sample was combined with a 500 μl of a DCA stock in phosphate buffer (Solution (A)), and an appropriate amount of KI (Solution (B)). Solution (A) contained 10 mM DCA, which was diluted from a 9.93 M DCA stock solution in 0.5 M phosphate, pH 8.5. Solution (B) contained 1 mM KI, which was diluted from a 400 mM stock solution. The vials were reacted overnight and 50 μl of the reaction sample was combined with 950 μl extractant before GC-MS analysis for detection of DCIM. FIG. 15 is a graph of the DCIM concentration versus the reaction coordinate, indicating that DCIM concentration increases linearly with time, in agreement with Michaelis Menten kinetics.

### Example 9. Pichia Pastoris Glycerol Batch and Fed-Batch Fermentation

Following the Example below, the enzyme ciVCPO from Curvularia inaequalis in Pischia pastoris was produced and purified to homogeneity, with typical results of 100-500 mg/l of more than 75-95% pure ciVCPO enzyme.

### Inoculum Seed Flask Preparation

One 250 mL baffled shake flask containing 25 mL of BMGY medium was inoculated with a single colony from an agar plate with the Pichia pastoris ciVCPO. The shake flask was incubated at 30° C. and 250 RPM in an incubator shaker for 24 hours. A second 2×500 ml baffled shake flask containing 150 mL of BMGY medium was inoculated with 5 mL (OD₆₀₀ around 0.2-0.4) of culture from the 250 mL baffled shake flask and incubated at 30° C. and 250 RPM in an incubator shaker for 24 hours.

### Glycerol Batch Phase

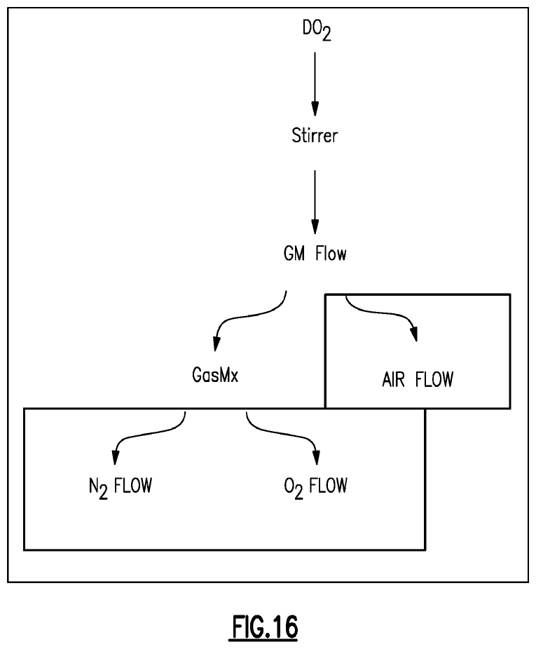

The pH probe, internal and external peristaltic pumps was first calibrated in the EVA software. The Bioreactor containing 3 L Basal Salts medium (FM22) plus 4% v/v glycerol and 0.6 ml Antifoam C was autoclaved at 121° C. for 30 minutes. After sterilization and cooling, 4 ml/liter PTM1 salts of total volume (9 ml) and Ampicillin was added aseptically. A 10 ml initial sample was taken. The O₂ probe (1000 rpm, 1 vvm air until stable reading) was calibrated and autozeroed OD₆₀₀ (Turbidity), and the tubes were filled with ammonium hydroxide (25%), phosphoric acid (10%), antifoam, and feeds. The temperature was set to 30° C., with agitation and aeration Min & Set values→>800 rpm, 0.5 vvm air|1.5 L/min, pO₂=40% v/v, and the pH of the medium was adjusted to 5.0 (for better salt solubility) with 25% ammonium hydroxide. The dissolved oxygen (DO) cascade is described in FIG. 16. The fermenter was ionoculated with approximately 5-10% initial fermentation volume from the culture generated in the inoculum shake flasks. the seed culture was spun down and redissolved in 50 ml BMGY medium or water, and 60 ml was added to a syringe with pink nail. The batch culture was allowed to grow until the glycerol was completely consumed (14 to 20 hours). This was indicated by an O₂ spike (pO₂>85% and Time>14 hours). Samples were analyzed for cell growth (OD600, cell wet weight (CWW), dry cell weight), ethanol, glycerol, and methanol concentrations, and enzyme activity. The cell pellets and supernatant of each sample were frozen at −80° C. for later analysis if not analyzed immediately.

### Sample Preparation

A 5 ml sample was transferred with high accuracy to a pre-weighed 15 ml falcon tube. 1 ml was removed and poured into a 1.5 ml Eppendorf tube. 0.1 ml of this sample was used for OD₆₀₀, 0.1<Abs<0.8. The remainder was spun down (0.9 ml) and the supernatant was transferred to a new 1.5 ml Eppendorf tube for the EtOH, MeOH, and Glycerol assays. If the sample was after induction, the paste was placed of the 0.9 ml in −80° C. for VCPO activity assay after breaking the cells with the yeast bug buster cell lysis buffer. The tube was spun down, washed with water, and spun down again, then the pellet was decanted and weighed. The CWW was calculated for the 4 ml cell culture and for CWW g/L. For cell dry weight (CDW): the pellet was washed again by resuspension and centrifugation, and the washed pellet was finally resuspended to make a final volume of 4 mL (initial). 3 mL of this suspension was transferred to a pre-weighed aluminum weighing dish, dried at 80° C. in an oven overnight for 16 hours, and weighed again. The dry mass of each sample was calculated to derive CDW.

### Glycerol Fed-Batch Phase

Once all the glycerol was consumed from the batch growth phase, a glycerol feed was initiated to increase the cell biomass under limiting (carbon) conditions. DO spikes were used to ensure the glycerol is limited. The glycerol fed-batch was initiated, after a spike in the DO was observed, with a 50% w/v glycerol feed containing 12 ml PTM1 salts per liter of glycerol feed. A constant feed rate of 20 ml/h*L (60 ml/h) initial fermentation volume was used. Glycerol feeding was carried out until the desired cell density was reached. A cellular yield of 230 g/L wet cells should be achieved at around 22 hours or 6 hours of feed. At the end of this stage, no appreciable recombinant enzyme is produced.

### Methanol Fed-Batch Phase of MutS Pichia Strain

All of the glycerol needs to be consumed before starting the methanol feed to fully induce the AOX1 promoter on methanol. Methanol was introduced slowly to adapt the culture to growth on methanol. The methanol feed rate was adjusted to maintain an excess of methanol in the medium which did not exceed 0.3%, as determined by GC/MS.

### The Methanol Induction Phase of the MutS Strain.

The temperature was lowered to 28° C., the glycerol feed rate was ramped down to 0 g/L *h (26.1% (60 ml/h)→0% in 2 h), and the culture was induced. The 2 L induction feed containing 50% v/v methanol, 12 ml/L PTM1 salts (24 ml), and 0.0075% v/v (0.15 ml) of antifoam 204, was initiated at 2 ml/h*L of initial fermentation volume for the first two hours. It was then ramped up from 2 ml/h*L to 6 ml/h*L in 12 hours and then maintained for the duration of the fermentation. The vessel was then harvested after 4 days on methanol.

### Enzyme Recovery: Cell Disruption

The total cell pellet was resuspended in water with a ratio of 1:4. 200 ml of this suspension was then added to the 500 ml Beckman centrifuge tubes (will be around 50 ml of cell paste), and centrifuged at 4000 g for 5 min at 4° C. The water was removed, leaving 50 mL of cell paste in each Beckman centrifuge tube. 50 ml beads, 200 ml binding buffer (50 mM Tris-HCl, pH=8.0) Protease inhibitor cocktail was added to 50 ml of the cell paste. The mixture was processed for 10 minutes to homogenize. Disrupted yeast appear dark under the microscope after this step. Next, the mixture was centrifuged, and the supernatant was collected and placed on ice. The paste was with 40 ml binding buffer, centrifuged, and combined with the previous supernatant. The tubes containing the cell lysate were centrifuged at 75,600 g (JA-25.50, 8×30 ml max, polycarbonate) for 15 min at 4° C. to remove insoluble cell debris, and collect the clear lysate. The lysate was then syringe filtered before purification.

### Enzyme Recovery: Purification

A thermal treatment of the crude extract (e.g. 55° C. for 45 min) and a classic affinity purification (His Tag) was employed for enzyme purification. The binding buffer was 0.1M Tris-HCl pH, the elution buffer was the same as binding buffer with 500 mM Imidazole added.

### Enzyme Characterization: SDS-PAGE Electrophoresis

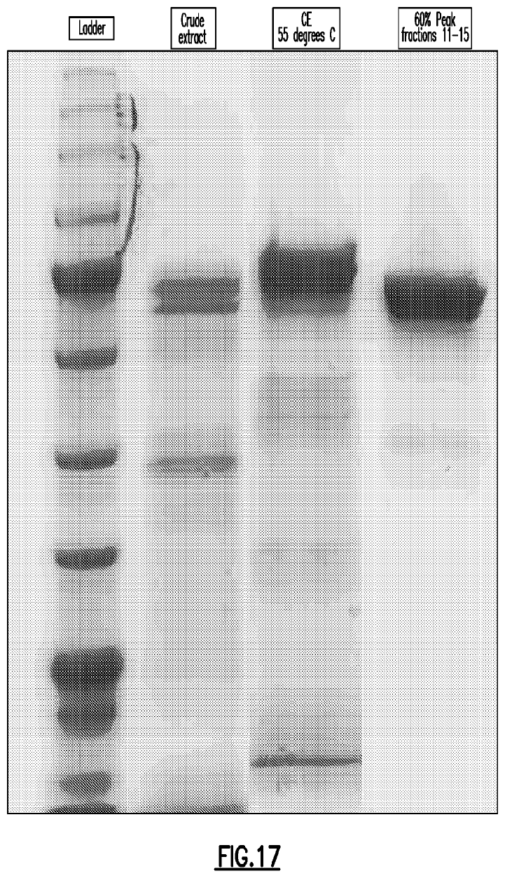

5 μg of pure protein or 10-20 μg mixture was added into each well. In an Eppendorf, 200 μl sample buffer was mixed with 2 μl fresh DTT 10 mM (stock is 1M), heated immediately at 90° C. for 5 min on a thermoblock, and then put on ice or frozen. The running buffer for SDS-Page contained 10× Tris/Glycine/SDS (900 ml mili-Q water+100 ml 10× Tris/Glycine/SDS) The staining buffer used was one-step blue. FIG. 17 is an SDS-PAGE of ladder, crude extract, crude extract after heat treatment, and 60% pool HisTap crude peak fractions.

### Buffer Exchange & Concentrate

The enzyme peak was selected, and the subsample was buffer exchanged with the desired buffer against a 20K MWCO dialyzer membrane. The samples were then frozen in liquid nitrogen and stored at −80° C.

### Enzyme Characterization: Activity Assay

$$MCD assay ( 292 nm ) , ε = 20 {mM}^{- 1} * {cm}^{- 1}$$

To a quartz cuvette was added in order: 2900 μl buffer (phosphate 50 mM pH 7), 50 μl 3M KBr, 20 μl of 10 mM MCD 4 μl 10 mM vanadium and 8 μl 300 mM H₂O₂. 20 uL was then added to a UV-Vis for reading at 292 nm every 6 seconds for 1 min.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A method of modulating production of a small halogenated organic compound with a peroxidase enzyme, comprising:
(i) providing a small organic compound (e.g., acetyl acetone);
(ii) contacting the small organic compound with a peroxidase (e.g., a VHPO) to form a reaction mixture under conditions sufficient to produce a small halogenated organic compound;
(iii) evaluating the small halogenated organic compound produced; and thereby modulating production of a small halogenated organic compound.

2. The method of claim 1, wherein the peroxidase is a haloperoxidase.

3. The method of claim 2, wherein the haloperoxidase is a vanadium haloperoxidase (VHPO).

4. The method of claim 3, wherein the VHPO is a vanadium chloroperoxidase (VCPO), vanadium bromoperoxidase (VBPO), or vanadium iodoperoxidase (VHPO).

5. The method of any one of claims 3-4, wherein the VHPO is a VBPO.

6. The method of claim 1, wherein the peroxidase is an algal haloperoxidase (e.g., derived from an algal species), a fungal haloperoxidase (e.g., derived from a fungal species), or a cyanobacterial haloperoxidase (e.g. derived from a cyanobacteria).

7. The method of claim 1, wherein the peroxidase is a fungal haloperoxidase (e.g., derived from a fungal species).

8. The method of claim 1, wherein the peroxidase is derived from an organism selected from Curvularia inaequalis, Halomicronema hongdechloris, Moorea bouillonii, Trichodesmium erythraeum, Aphanocapsa montana, Lyngbya confervoides, Synechococcus sp. PCC7335, and Corallina officinalis.

9. The method of claim 1, wherein the peroxidase is produced in a host cell microorganism.

10. The method of claim 9, wherein the host cell microorganism is selected from Pichia pastoris, Aspergillus niger, Saccharomyces cerevisiae, or Escherichia coli.

11. The method of any one of claims 9-10, wherein expression of the peroxidase produced in the host cell microorganism is increased by about 1.5-fold, 2-fold, 3-fold, 4-fold, 5-fold, 6-fold, 7-fold, 8-fold, or 10-fold, e.g., over a peroxidase produced in its native host.

12. The method of claim 1, wherein the amino acid sequence of the peroxidase is selected from an amino acid sequence listed in Table 1.

13. The method of claim 1, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from the list in Table 1.

14. The method of claim 1, wherein the peroxidase is a sequence selected from any one of SEQ ID NOs. 1-50.

15. The method of claim 1, wherein the amino acid sequence of the peroxidase has at 1, 2, 3, 4, 5, or 6 amino acid substitutions relative to an amino acid sequence selected from any one of SEQ ID NOs. 1-50.

16. The method of claim 1, wherein the small organic compound has a structure of Formula (Y), e.g., described herein.

17. The method of claim 1, wherein the small organic compound comprises 1, 2, 3, 4, 5, 6, 7, or 8 carbon atoms.

18. The method of claim 1, wherein the small organic compound comprises a ketone or aldehyde.

19. The method of claim 1, wherein the small halogenated organic compound comprises has a structure of Formula (Z), e.g., described herein.

20. The method of claim 1, wherein the small halogenated organic compound is chlorinated; brominated; iodinated; chlorinated and iodinated; chlorinated and brominated; brominated and iodinated; or chlorinated, brominated, and iodinated.

21. The method of claim 1, wherein the small halogenated organic compound is brominated.

22. The method of claim 1, wherein the small halogenated organic compound comprises 1, 2, 3 halogen atoms.

23. The method of claim 1, wherein the small halogenated organic compound comprises 1, 2, 3 bromine atoms.

24. The method of claim 1, wherein the small halogenated organic compound comprises an acetone moiety.

25. The method of claim 1, wherein the small halogenated organic compound comprises dibromoacetone, bromoacetone, bromopentanedione, bromoform, or tribromoacetone.

26. The method of claim 1, wherein the small halogenated organic compound comprises dichloroiodomethane, dibromochloromethane, 1,1-dibromoacetone, bromoacetone, 3-bromo-2,4-pentanedione, bromoform, 1,1,3-tribromoacetone, or 1,1,1-tribromoacetone.

27. The method of claim 1, further comprising providing a peroxide source, e.g. H₂O₂ or PAA, and a halogen source, e.g. halogenated salts such as KBr, NaCl or Kl.

28. The method of claim 1, wherein the conditions sufficient to product a small halogenated organic compound comprise one or more of:
(a) temperature between 10° C. to 85° C.
(b) pH between 4-10; and
(c) an ionic strength between 0.1 mM to 4 M.

29. The method of claim 1, further comprises reducing methane production by at least 5%, 10%, 15%, 20%, 25% 30%, 35%, 40%, 50%, 55%, 60%, 65%, 70%, 75%, 80%, 85%, 90%, 95%, 99%, or more.

30. The method of claim 29, wherein the methane production is reduced by between 10-⁷⁵%.

31. A method for reducing production of methane in a rumen community, comprising:
(i) providing a small organic compound (e.g., acetyl acetone);
(ii) contacting the small organic compound with a peroxidase (e.g., a VHPO) to form a reaction mixture under conditions sufficient to produce a small halogenated organic compound;
(iii) separating the small halogenated organic compound from the reaction mixture;
(iv) providing the small halogenated organic compound to a rumen community under conditions sufficient to reduce the production of methane.

32. The method of claim 31, further comprising acquiring a value for the level of methane (a) prior to providing the peroxidase or (b) after providing the peroxidase.

33. The method of claim 32, comprising (a).

34. The method of claim 32, comprising (b).

35. The method of claim 32, comprising (a) and (b).

36. The method of claim 31, wherein methane production is reduced by at least 5%, 10% 0.15%, 20%, 25% 30%, 35%, 40%, 50%, 55%, 60%, 65%, 70%, 75%, 80%, 85%, 90%, 95%, 99%, or more.

37. The method of claim 31, wherein the methane production is reduced by between 10-75%.

38. The method of claim 31, wherein the peroxidase is a haloperoxidase.

39. The method of claim 38, wherein the haloperoxidase is a vanadium haloperoxidase (VHPO).

40. The method of claim 39, wherein the VHPO is a vanadium chloroperoxidase (VCPO), vanadium bromoperoxidase (VBPO), or vanadium iodoperoxidase (VHPO).

41. The method of claim 31, wherein the peroxidase is produced in a host cell microorganism, e.g., selected from Pichia pastoris, Aspergillus niger, or Escherichia coli.

42. The method of claim 41, wherein expression of the peroxidase produced in the host cell microorganism is increased by about 1.5-fold, 2-fold, 3-fold, 4-fold, 5-fold, 6-fold, 7-fold, 8-fold, or 10-fold, e.g., over a peroxidase produced in its native host.

43. The method of claim 31, wherein the amino acid sequence of the peroxidase is selected from an amino acid sequence listed in Table 2.

44. The method of claim 31, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from the list in Table 2.

45. The method of claim 31, wherein the peroxidase has at least 75% sequence identity (e.g., at least 80%, 85%, 90%, 91%, 92%, 93%, 94%, 95%, 96%, 97%, 98%, 99%, 99%, or 99.5% sequence identity) to a peroxidase sequence selected from SEQ ID NOs: 1-50.

46. The method of claim 31, wherein the small organic compound has a structure of Formula (Y), e.g., described herein.

47. The method of claim 31, wherein the small organic compound comprises 1, 2, 3, 4, 5, 6, 7, or 8 carbon atoms.

48. The method of claim 31, wherein the small organic compound comprises a ketone or aldehyde.

49. The method of claim 31, wherein the small halogenated organic compound comprises has a structure of Formula (Z), e.g., described herein.

50. The method of claim 31, wherein the small halogenated organic compound is chlorinated; brominated; iodinated; chlorinated and iodinated; chlorinated and brominated; brominated and iodinated; or chlorinated, brominated, and iodinated.

51. The method of claim 31, wherein the small halogenated organic compound is brominated.

52. The method of claim 31, wherein the small halogenated organic compound comprises 1, 2, 3 halogen atoms.

53. The method of claim 31, wherein the small halogenated organic compound comprises 1, 2, 3 bromine atoms.

54. The method of claim 31, wherein the small halogenated organic compound comprises an acetone moiety.

55. The method of claim 31, wherein the small halogenated organic compound comprises dibromoacetone, bromoacetone, bromopentanedione, bromoform, or tribromoacetone.

56. The method of claim 31, wherein the small halogenated organic compound comprises dichloroiodomethane, dibromochloromethane, 1,1-dibromoacetone, bromoacetone, 3-bromo-2,4-pentanedione, bromoform, 1,1,3-tribromoacetone, or 1,1,1-tribromoacetone.

57. The method of claim 31, further comprising providing a peroxide source, e.g. H₂O₂ or PAA, and a halogen source, e.g. halogenated salts such as KBr, NaCl or KI.

58. The method of claim 31, wherein the conditions sufficient to product a small halogenated organic compound comprise one or more of:
(a) temperature between 10° C. to 85° C.
(b) pH between 4-10; and
(c) an ionic strength between 0.1 mM to 4 M.
